(19) United States
(12) Patent Application Publication
(10) Pub. No.: US 2013/0022980 A1
(43) Pub. Date: Jan. 24, 2013

(54) RNA- AND DNA-COPYING ENZYMES

(71) Applicants: Robert Michael Nelson, Wellesley, MA (US); Thomas W. Schoenfeld, Madison, WI (US); David A. Mead, Middleton, WI (US)

(72) Inventors: Robert Michael Nelson, Wellesley, MA (US); Thomas W. Schoenfeld, Madison, WI (US); David A. Mead, Middleton, WI (US)

(73) Assignee: Lucigen Corporation

(21) Appl. No.: 13/147,446

(22) PCT Filed: Feb. 4, 2010
(86) PCT No.: PCT/US2010/023233

§ 371 Date: Mar. 15, 2012

Related U.S. Application Data
(60) Provisional application No. 61/149,904, filed on Feb. 4, 2009.

(51) Int. Cl. C12N 9/96; C12Q 1/68; C07H 21/04; C12P 19/34

(52) U.S. Cl. 435/6.12; 435/188; 435/91.5; 435/6.1; 536/23.2; 435/91.2

(57) ABSTRACT

The present invention is directed to DNA polymerase fusion proteins with increased processivity and nucleic acid affinity. The invention includes a fusion protein comprising a nucleic acid-binding domain fused to a polymerase domain. The nucleic acid binding domain contains at least one nucleic acid binding motif, such as a DNA-binding motif or an RNA-binding motif. The nucleic acid binding domain preferably embodies an oligonucleotide/oligosaccharide binding (OB) fold, among other conformations. The invention further includes methods of synthesizing nucleic acids using the fusion proteins described herein.

### CROSS REFERENCE TO RELATED APPLICATIONS

This application claims priority under 35 USC §119(e) to U.S. Provisional Patent Application 61/149,904 filed Feb. 4, 2009, the entirety of which is incorporated herein by reference.

### FIELD OF THE INVENTION

This invention provides fusion proteins comprising a nucleic acid-binding domain and a polymerase domain, and methods for using such fusion proteins in nucleic acid synthesis reactions.

### BACKGROUND

DNA polymerases synthesize DNA molecules that are complementary to all or a portion of a nucleic acid template, such as a DNA or an RNA template. Upon hybridization of a primer to a nucleic acid template, DNA polymerases add nucleotides to the 3′ hydroxyl end of the primer in a template-dependent manner. Thus, in the presence of deoxyribonucleoside triphosphates (dNTPs) and a primer, a polymerase can synthesize a new DNA molecule complementary to all or a portion of one or more nucleic acid templates.

Processivity is a measurement of the number of nucleotides added to a nucleic acid strand by a polymerase per nucleic acid binding event. DNA polymerases having low processivity, such as the Klenow fragment of DNA polymerase I of E. coli, will dissociate after about 5-40 nucleotides are incorporated. Other polymerases, such as T7 DNA polymerase, are able to incorporate many thousands of nucleotides prior to dissociating. Such processivity can be measured as described by Tabor et al., JBC 262, 16212 (1987). Increased polymerase processivity is advantageous in biochemical reactions requiring copying or amplification nucleic acid, such as polymerase chain reaction (PCR) (U.S. Pat. No. 4,965,188 to Mullis et al.) and DNA sequencing (U.S. Pat. No. 4,795,699 to Tabor).

### SUMMARY OF THE INVENTION

The current invention generally provides fusion proteins comprising a nucleic acid-binding domain and a polymerase domain for increased processivity in nucleic acid synthesis reactions. The fusion proteins described herein enhance processivity by increasing the affinity of the polymerase to the nucleic acid or increasing the stability of the polymerase/nucleic acid complex.

One version of the invention includes a fusion protein comprising a first polypeptide domain operationally connected to or directly linked to a second polypeptide domain wherein the first polypeptide domain comprises an oligonucleotide/oligosaccharide binding (OB) fold and at least one RNA binding motif and wherein the second polypeptide domain comprises a polymerase domain. The RNA binding motif may include a sequence such as GYGFI, VFVHW, or VFVHF. The RNA binding motif may be contained on beta sheet β2 or beta sheet β3 of the OB fold.

In another version of the invention, the first polypeptide domain of the fusion protein includes at least two RNA binding motifs. A first of the at least two RNA binding motifs may be contained on beta sheet β2 of the OB fold and a second of the at least two RNA binding motifs may be contained on beta sheet β3 of the OB fold.

In another version of the invention, the first polypeptide domain of the fusion protein includes a DNA binding motif. The DNA binding motif may be between beta sheets β3 and β4 of the OB fold. The DNA binding motif may include a sequence such as AIEM, AIQG, AIGN, VGKM, VGKA, AGKA, or LAPKGRKGVKI.

In some versions of the invention, the first polypeptide domain of the fusion protein is thermostable.

In some versions of the invention, the first polypeptide domain is at least 60% identical to a sequence selected from the group consisting of SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, and SEQ ID NO: 70.

In another version of the invention, the first polypeptide domain is at least 95% identical to SEQ ID NO: 70.

In some versions of the invention, the polymerase domain is a DNA-dependent DNA polymerase. In other versions, the polymerase domain is an RNA-dependent DNA polymerase.

In some versions of the invention, the polymerase domain is a Klenow fragment of a DNA polymerase.

In some versions of the invention, the second polypeptide domain is at least 60% identical to a sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, and SEQ ID NO: 24.

Some versions of the invention further include a linker between the first polypeptide domain and the second polypeptide domain.

In another version of the invention, the fusion protein further includes a third polypeptide domain operationally connected to the first polypeptide domain and the second polypeptide domain or directly linked to the first polypeptide domain or the second polypeptide domain, wherein the third polypeptide domain comprises at least one RNA binding motif and/or at least one DNA binding motif. The third polypeptide domain may comprise an OB fold. The third polypeptide domain may be at least 80% identical to a sequence selected from the group consisting of SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, and SEQ ID NO: 70.

The invention further provides a nucleic acid that encodes a fusion protein as described herein, in addition to vectors, host cells, and kits comprising the nucleic acid.

The invention also provides a method of synthesizing a nucleic acid comprising contacting a nucleic acid template with a fusion protein as described herein. The contacting may be performed in any procedure requiring synthesis of a nucleic acid from a template. Such procedures include but are not limited to measuring levels of mRNA in a cell extract, sequencing a nucleic acid, synthesizing DNA polymers, reverse transcribing RNA polymers to produce complementary DNA (cDNA), amplifying DNA in a polymerase chain reaction (PCR), amplifying DNA in an isothermal nucleotide amplification reaction, and reverse transcribing RNA and amplifying DNA in a one-tube, one-enzyme reverse transcription-polymerase chain reaction (RT-PCR).

The fusion proteins described herein more efficiently copy DNA to allow, among other things: (1) PCR amplification of longer sequences of DNA; (2) PCR amplification of sequences that are difficult to amplify by conventional means due to high or low content of guanosine or cytosine residues or secondary structure; (3) PCR amplification in a shorter time period; (4) nucleotide sequence analysis of sequences that are difficult due to high or low content of guanosine and cytosine residues or secondary structure; and (5) more efficient isothermal amplification of DNA by strand displacement amplification, loop mediated amplification, rolling circle and other methods.

The fusion proteins described herein also reverse transcribe RNA into complementary DNA (cDNA) and alleviate RNA secondary structure. When thermostable RNA- and DNA-binding domains are fused to thermostable reverse transcriptases, the invention provides for novel fusion enzymes which catalyze reverse transcription of RNA into cDNA at temperatures above 45° C. Under such high-temperature reaction conditions (45° to 75° C.), RNA secondary structure is effectively disrupted. As a result, the reaction yield and rate of reverse transcription of RNA is increased, as compared to RT reactions at lower temperatures (Myers and Gelfand, 1991; Mizuno et al., 1999; Yasukawa et al., 2008).

Some versions of the fusion proteins described herein provide the ability to enzymatically copy RNA and amplify the resulting cDNA with a single enzyme. The need to transfer first-step reverse transcription (RT) reaction products into a second-step DNA amplification reaction (such as PCR; U.S. Pat. No. 4,965,188 to Mullis et al.) is obviated. Instead, the same polymerase enzyme is employed for both RNA copying and DNA amplification.

Furthermore, if the polymerase and nucleic acid-binding domains are thermostable, then one-tube, one-enzyme RT-PCR can be carried out at elevated temperatures (45 to 75° C.). High temperature one-tube, one-enzyme RT-PCR offers major technical advantages for nucleic acid-based medical diagnostic tests and high-throughput analyses of gene expression. These advantages include improved reaction yield, speed, simplicity, ease-of-use, ease-of-manufacturing, cost, and avoidance of cross-contamination.

The objects and advantages of the invention will appear more fully from the following detailed description of the invention and the accompanying drawings.

### BRIEF DESCRIPTION OF THE DRAWINGS

FIG. 1A depicts the amino acid sequence of Thermotoga maritima Cold shock protein (TmCsp) (SEQ ID NO: 26) with residues corresponding with the five (3-sheets, two RNA-binding motifs (RNP-1 and RNP-2), and the minor groove DNA-binding loop indicated.

FIG. 1B is a diagrammatic representation of an N-terminal fusion of TmCsp to 3173 Pol via a flexible hinge.

FIG. 2A is an amino acid sequence alignment of three OB-fold nucleic acid-binding proteins: Sac7d-V26/A29 mutant (SEQ ID NO: 34), SshCren7 (SEQ ID NO: 38), and TmCsp (SEQ ID NO: 26). The five β-sheets and the DNA-binding loops between beta sheets β3 and β4 on each of these proteins are shown. Also shown are the RNA-binding motifs (RNP-1 and RNP-2) on beta sheets β2 and β3 of TmCsp.

FIG. 2B depicts a schematic showing the secondary structure of Sac 7d-V26/A29 with the DNA-binding loop between beta sheets β3 and β4.

FIG. 2C depicts a schematic showing the secondary structure of SshCren7 with the DNA-binding loop between beta sheets β3 and β4.

FIG. 2D depicts a schematic showing the secondary structure of TmCsp with the RNA-binding motifs (RNP-1 and RNP-2) on beta sheets β2 and β3 and the DNA-binding loop between beta sheets β3 and β4.

FIG. 3A is an amino acid sequence alignment of two OB-fold nucleic acid-binding proteins: TmCsp (SEQ ID NO: 26) and Sac7d-V26/A29 mutant (SEQ ID NO: 34). The five β-sheets and the DNA-binding loops between beta sheets β3 and β4 on each of these proteins are shown. Also shown are the RNA-binding motifs (RNP-1 and RNP-2) on beta sheets β2 and β3 of TmCsp. Sac7d-V26-A29 does not contain the RNP-1 or RNP-2 RNA-binding motifs.

FIG. 3B is a diagrammatic representation of an N-terminal fusion of RNA-binding TmCsp to 3173 Pol via a flexible hinge.

FIG. 3C is a diagrammatic representation of an N-terminal fusion of RNA-binding TmCsp and a C-terminal fusion of DNA-binding Sac7d (mutant) to 3173 Pol via flexible hinges.

FIG. 3D is a diagrammatic representation of a C-terminal fusion of RNA- and DNA-binding TmCsp to 3173 Pol via a flexible hinge.

FIG. 4A is an amino acid sequence alignment of three OB-fold nucleic acid-binding proteins: Sac7d-V26/A29 mutant (SEQ ID NO: 34), TmCsp (SEQ ID NO: 26), and a chimeric protein comprising a Sac7d-V26/A29 sequence with the RNP-1 and RNP-2 RNA-binding motifs of TmCsp (SEQ ID NO: 70). The five β-sheets and the DNA-binding loops between beta sheets β3 and β4 on each of these proteins are shown. Also shown are the RNA-binding motifs (RNP-1 and RNP-2) on beta sheets β2 and β3 of TmCsp and the chimera.

FIG. 4B is a schematic showing the secondary structure of the chimeric protein depicted in FIG. 4A.

FIG. 4C is a diagrammatic representation of an N-terminal fusion of a chimeric protein depicted in FIGS. 4A and B to PyroPhage 3173 Pol via a flexible hinge.

FIG. 5 shows gel shift assay results demonstrating affinity of an SSB-PyroPhage 3173 DNA polymerase fusion protein for nucleic acid. Lane 1: DNA in absence of fusion protein. Lane 2: DNA in presence of protein. Lane 3: DNA markers ranging from 250 to 10,000 bp.

FIG. 6 shows a comparison of conventional Taq DNA polymerase (SEQ ID NO: 4) (lanes 2, 3, 6, 7) versus a fusion protein comprising Taq Pol Δ289 (SEQ ID NO: 6) fused to Sac7d-V26/A29 (SEQ ID NO: 34) at its N-terminus (lanes 4, 5, 8, 9) in amplifying genomic DNA targets through PCR in the presence of whole blood. Lanes 1 and 10 show DNA markers ranging from 250 to 10,000 bp.

FIGS. 7A and 7B show a comparison of Taq DNA polymerase (SEQ ID NO: 4) (FIG. 7A) versus a fusion protein comprising Taq Pol Δ289 (SEQ ID NO: 6) fused to Sac7d-V26/A29 (SEQ ID NO: 34) at its N-terminus (FIG. 7B) in amplifying randomly picked clones from a library of Cellvibrio gilvus inserts in an expression vector through colony PCR. Lanes 1 and 50 in FIGS. 7A and 7B show DNA markers ranging from 250 to 10,000 bp.

FIG. 8 shows a comparison of PyroPhage Exo-DNA polymerase (SEQ ID NO: 18) (lane 2), PyroPhage Exo-DNA polymerase with the VA Sac7d protein (SEQ ID NO: 34) fused to the amino terminus of PyroPhage Exo-(lane 3), and TmCsp (SEQ ID NO: 26) fused to the amino terminus of PyroPhage Exo-(lane 4) in PCR amplification of DNA. Lane 1 shows DNA markers ranging from 250 to 10,000 bp.

FIG. 9 shows primer extension and gel shift assays of various polymerases with and without Tbr single strand binding (SSB) protein fused thereto. Lanes 1 and 14 show DNA markers ranging from 250 to 10,000 bp.

### DETAILED DESCRIPTION OF THE INVENTION

### Abbreviations and Definitions

aa: Amino acid.

cDNA: Complementary deoxyribonucleic acid, the reaction product after reverse transcription of RNA.

Cren7: A nucleic acid-binding protein isolated from Crenarchaeota which is an OB-fold protein comprised of 5 β-sheets.

Csp: Cold shock protein, a member of the OB-fold class of proteins.

DNA: Deoxyribonucleic acid.

DNA-Binding Motif: An amino acid sequence that binds DNA. DNA-binding motifs include but are not limited to the dsDNA-binding loops between the β3 and β4 beta sheets and the ssDNA binding sites on OB-fold proteins.

dNTP: Deoxynucleotide triphosphate; dATP, dCTP, dGTP, and dTTP.

Domain: A portion of a protein sequence which carries out ligand binding, catalytic activity, or has a stabilizing effect of the structure of a protein.

E.C. 2.7.7.49: Enzyme Committee of the International Union of Biochemistry and Molecular Biology designation of an RNA-dependent DNA polymerase enzyme (reverse transcriptase), which catalyzes RNA template-directed extension of the 3′ end of a DNA strand by one nucleotide at a time, and requires an RNA or DNA primer.

E.C. 2.7.7.7: Enzyme Committee of the International Union of Biochemistry and Molecular Biology designation of a DNA-dependent DNA polymerase enzyme, which catalyzes DNA template-directed extension of the 3′ end of a DNA strand by one nucleotide at a time, and requires a primer, which may be either DNA or RNA.

Enzyme: A catalyst, normally a protein, which increases the rate of a chemical reaction.

mRNA: messenger RNA.

Nucleic Acid-Binding Domain: A protein sequence or portion of a protein sequence which facilitates binding to RNA and/or DNA.

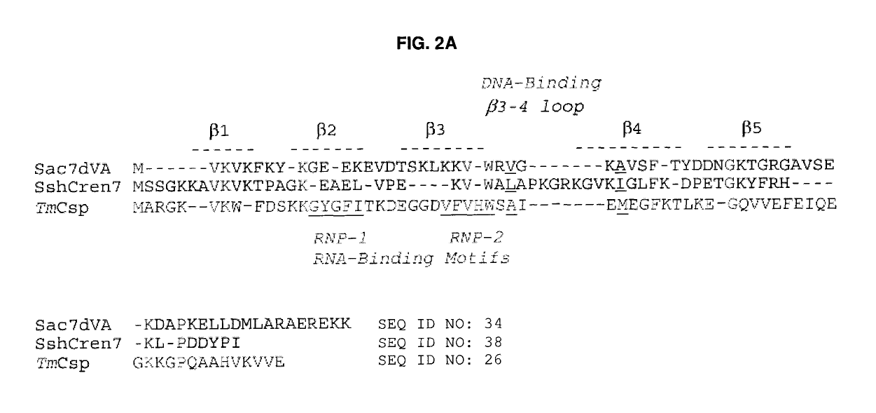

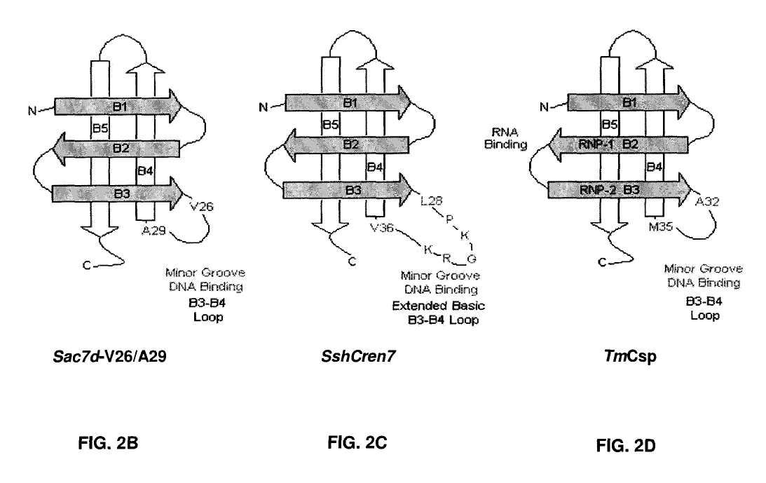

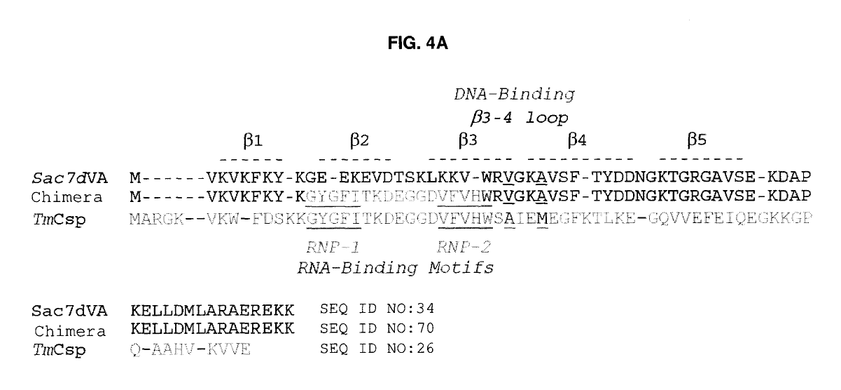

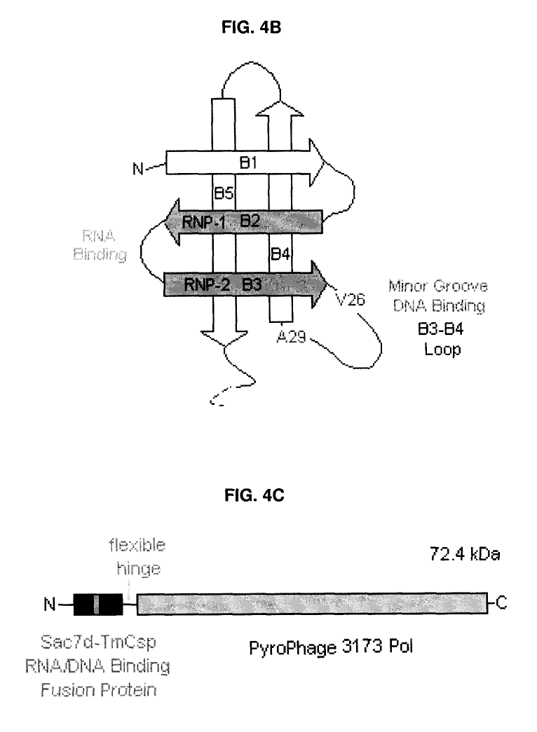

OB-fold Protein: Oligonucleotide/oligosaccharide binding protein folded in a conserved 5-stranded β sheet motif coiled to form a closed β-barrel, as first described by Murzin (1993). See FIGS. 2B, 2C, 2D, and 4C.

Operationally Connected or Linked: When referring to two or more protein or nucleic acid domains means that upstream domains function as noted with respect to downstream domains and vice-versa, even though the two domains are not necessarily directly linked to one another.

PCR: the polymerase chain reaction, as originally described by Saiki et al. (1985) and U.S. Pat. No. 4,965,188 to Mullis et al.

Polymerase: an enzyme which catalyses the primer-dependent copying of a nucleic acid template (DNA or RNA) from dNTPs.

Processivity: the number of nucleotides incorporated per nucleic acid binding event.

qPCR: quantitative PCR, in which the amount of amplified nucleic acid is measured after amplification using the polymerase chain reaction.

Reverse Transcriptase (RT): a polymerase which catalyses the enzymatic copying of RNA into complementary DNA.

Reverse Transcription: The synthesis of a DNA strand complementary to an RNA target.

RNA: ribonucleic acid.

RNA-Binding Motif: An amino acid sequence that binds RNA. RNA-binding motifs include but are not limited to the RNA binding sites on the β2 and β3 beta sheets on OB-fold proteins.

RT-PCR: reverse transcription of RNA into cDNA, followed by PCR amplification.

SSB: single-stranded DNA-binding protein.

ssDNA: single-stranded deoxyribonucleic acid.

ssRNA: single-stranded ribonucleic acid.

Thermotoga Maritima: A rod-shaped bacterium belonging to the order Thermotogales, originally isolated from geothermal heated marine sediment at Vulcano, Italy.

### DESCRIPTION

The present invention describes novel nucleic acid copying enzymes in which nucleic acid-binding domains, which bind to RNA and/or DNA, are fused to polymerases. These engineered fusion enzymes display higher affinity RNA-binding, improved ability to enzymatically copy RNA into cDNA, and enhanced performance in enzymatic DNA amplification reactions.

The invention provides for a fusion protein comprised of at least two domains: a nucleic acid-binding domain that binds to RNA and/or DNA; and a polymerase domain. In one embodiment, the nucleic acid polymerase is a DNA-dependent DNA polymerase. In another embodiment, the nucleic acid polymerase is an RNA-dependent DNA polymerase (i.e., a reverse transcriptase).

Fusion Proteins: A fusion protein of the current invention may be constructed with the nucleic acid-binding domain at the N-terminus and the polymerase domain at the C-terminus or vice-versa. Thus, a DNA construct encoding the fusion protein may comprise the nucleic acid-binding portion upstream (5′) of the polymerase portion or vice versa. Nucleic acid-binding genes are cloned upstream (or downstream) and in frame with a polymerase gene using methods well-known in the art of molecular biology (see e.g., Molecular Cloning, a Laboratory Manual, Cold Spring Harbor Laboratory Press, Cold Spring Harbor, N.Y., 1989). In some embodiments, the polymerase domain is fused to two nucleic acid binding domains, with a first nucleic acid-binding domain fused to the N-terminus of the polymerase and a second nucleic acid-binding domain fused to the C-terminus of the polymerase. The nucleic acid-binding domain and the polymerase domain may be immediately adjacent to each other, or may be separated by an amino acid linker. The amino acid linker may be 1, 2, 3, 4, 5, 6, 7, 8, 9, 10, 11, 12, 13, 14, 15, 16, 17, 18, 19, 20, 25, 30, 40, 50, 60, 70, 80, 90 or 100 or more amino acids in length. Suitable linkers for joining two domains in fusion proteins are well-known in the art. See, for example, U.S. Pat. No. 5,856,456 and U.S. Publication 2009/0221477. A preferred linker, as described herein, comprises the amino acid sequence GSAG (see SEQ ID NOS: 42, 44, 46, 48, 50, 52, 54, 56, 58, 60, 62, 64, 66, 68, and 72).

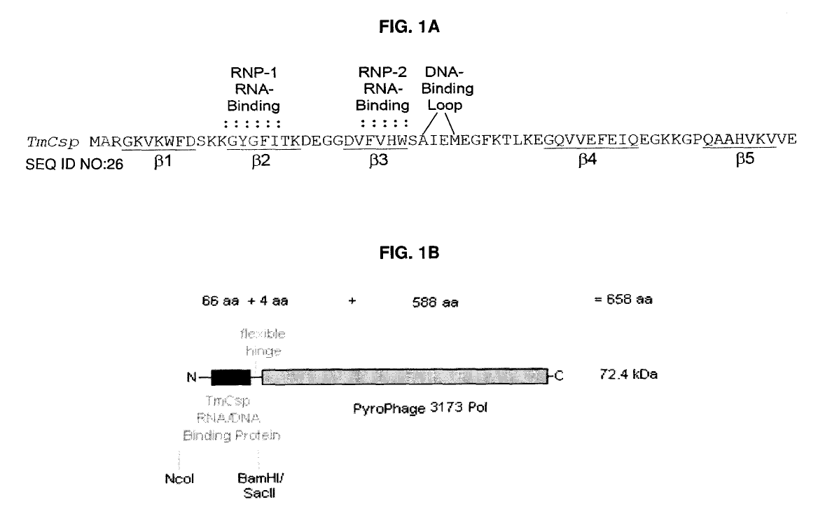

Exemplary fusion proteins of the present invention include: Sulfolobus acidocaldarius engineered nucleic acid-binding protein (Sac7d mutant AA) fused to 3173 DNA Polymerase Double Mutant D49A/F418Y (AA-3173 AY Pol; SEQ ID NO: 42); Sulfolobus acidocaldarius engineered nucleic acid-binding protein (Sac7d mutant VA) fused to 3173 DNA Polymerase Double Mutant D49A/F418Y (VA-3173 AY Pol; SEQ ID NO: 44); Thermotoga maritima engineered Cold shock protein (TmCsp) fused to 3173 DNA Polymerase Double Mutant D49A/F418Y (TmCsp-3173 AY Pol; SEQ ID NO: 46); Sulfolobus acidocaldarius engineered nucleic acid-binding protein (Sac7d mutant VA) fused to 3173 DNA Polymerase Mutant D49A (VA-3173 A Pol; SEQ ID NO: 48); Sulfolobus acidocaldarius engineered nucleic acid-binding protein (Sac7d mutant VA) fused to Wild Type 3173 DNA Polymerase (VA-3173 Pol; SEQ ID NO: 50); Sso7d fused to Thermus aquaticus DNA Polymerase F672Y 289 AA deletion mutant (Sso7d Taq Y Δ289 Pol; SEQ ID NO: 52); Sulfolobus acidocaldarius engineered nucleic acid-binding protein Sac7d mutant VA fused to Bacteriophage T4 DNA Polymerase Exonuclease-mutant (VA-T4 exo-Pol; SEQ ID NO: 54); Sulfolobus acidocaldarius engineered nucleic acid-binding protein Sac7d mutant VA fused to Exonuclease-Large Fragment (Klenow Fragment) of Escherichia coli DNA Polymerase I (Klenow exo-VA Pol; SEQ ID NO: 56); Sulfolobus acidocaldarius engineered nucleic acid-binding protein Sac7d mutant VA fused to Dictyoglomus turgidus 281 AA deletion exo-DNA Polymerase (Dtu exo-VA Pol; SEQ ID NO: 58); Exonuclease Minus Large Fragment (Klenow Fragment) of Escherichia coli DNA Polymerase I fused to Tbr SSB protein (Klenow exo-Tbs SSB Pol; SEQ ID NO: 60); Thermus brockianus Single Strand Binding protein fused to 3173 DNA Polymerase Double Mutant D49A/F418Y (3173 AY-SSB Pol; SEQ ID NO: 62); Escherichia coli bacteriophage T4 DNA Polymerase exonuclease minus mutant fused to Tbr SSB protein (T4 exo-Tbr SSB Pol; SEQ ID NO: 64); 3173 DNA Polymerase Double Mutant D49A/F418Y C-terminally fused to Thermotoga maritima Cold shock protein (TmCsp) (3173 Pol AY-TmCsp; SEQ ID NO: 66); Thermotoga maritima engineered Cold shock protein (TmCsp) fused to 3173 DNA Polymerase Double Mutant D49A/F418Y fused to Sac7d mutant VA (TmCsp-3173 AY Pol-VA; SEQ ID NO: 68); and an N-terminal fusion of a chimeric nucleic acid-binding protein to 3173 Pol Double Mutant D49A/F418Y (SEQ ID NO: 72). See FIGS. 1B, 3B-3D, and 4C.

Polymerase Domain: The polymerase domain may include any polymerase known or discovered in the future capable of generating a nucleic acid polymer from a nucleic acid template. The polymerase preferably includes a DNA polymerase. In one embodiment, the polymerase is a DNA-dependent DNA polymerase. In another embodiment, the polymerase is an RNA-dependent DNA polymerase. In some versions, the polymerase domain is thermostable. Exemplary polymerases for use in the current invention include: Thermus thermophilus DNA polymerase (Tth Pol; SEQ ID NO: 2); Thermus aquaticus DNA Polymerase F672Y full length (Taq Pol Y; SEQ ID NO: 4); Thermus aquaticus DNA Polymerase F672Y 289 AA deletion mutant (Taq Pol Y Δ289; SEQ ID NO: 6); Bacteriophage T4 DNA Polymerase Exonuclease-mutant (T4 exo-Pol; SEQ ID NO: 8); Escherichia coli DNA Polymerase I Exonuclease-Large Fragment (Klenow Fragment) (Klenow exo-Pol; SEQ ID NO: 10); Avian Myeloblastosis Virus Reverse Transcriptase (AMV RT; SEQ ID NO: 12); Moloney Murine Leukemia Virus Reverse Transcriptase (MoMLV RT; SEQ ID NO: 14); 3173 Thermostable Phage DNA Polymerase (3173 Pol; SEQ ID NO: 16); 3173 Thermostable Phage DNA Polymerase E51A (3173 Pol; SEQ ID NO: 18); 3173 DNA Polymerase Double Mutant D49A/F418Y (3173 Pol AY; SEQ ID NO: 20); Dictyoglomus turgidus 281 AA deletion exo-DNA Polymerase (Dtu Pol; SEQ ID NO: 22); and Dictyoglomus thermophilum H-6-12 DNA Polymerase (Dth Pol; SEQ ID NO: 24).

DNA Polymerase (DNAP): A DNA polymerase is an enzyme that can add deoxynucleoside monophosphate molecules to the 3′ hydroxy end of a primer in a primer-template complex, and then sequentially to the 3′ hydroxy end of a growing primer extension product according to an RNA or DNA template that directs the synthesis of the polynucleotide. For example, a DNA polymerase can synthesize the formation of a DNA molecule complementary to a single-stranded DNA or RNA template by extending a primer in the 5′-to-3′ direction. DNAPs include DNA-dependent DNA polymerases and RNA-dependent DNA polymerases. A given DNAP may have more than one polymerase activity. For example, some DNA-dependent DNA polymerases, such as Taq, also exhibit RNA-dependent DNAP activity. DNAPs typically add nucleotides that are complementary to the template being used, but DNAPs may add non-complementary nucleotides (mismatches) during the polymerization or synthesis process. Thus, the synthesized nucleic acid strand may not be completely complementary to the template. DNAPs may also make nucleic acid molecules that are shorter in length than the template used. DNAPs have two preferred substrates: one is the primer-template complex where the primer terminus has a free 3′-hydroxyl group; the other is a deoxynucleotide 5′-triphosphate (dNTP). A phosphodiester bond is formed by nucleophilic attack of the 3′-OH of the primer terminus on the α-phosphate group of the dNTP and elimination of the terminal pyrophosphate. DNAPs can be isolated from organisms as a matter of routine by those skilled in the art, and can be obtained from a number of commercial vendors.

Some DNAPs are thermostable, and are not substantially inactivated at temperatures commonly used in PCR-based nucleic acid synthesis. Such temperatures vary depending upon reaction parameters, including pH, template and primer nucleotide composition, primer length, and salt concentration. Thermostable DNAPs include Thermus thermophilus (Tth) DNAP, Thermus aquaticus (Taq) DNAP, Thermotoga neopolitana (Tne) DNAP, Thermotoga maritima (Tma) DNAP, Thermotoga strain FjSS3-B.1 DNAP, Thermococcus litoralis (Tli or VENT™) DNAP, Pyrococcus furiosus (Pfu) DNAP, DEEPVENT™ DNAP, Pyrococcus woosii (Pwo) DNAP, Pyrococcus sp KOD2 (KOD) DNAP, Bacillus sterothermophilus (Bst) DNAP, Bacillus caldophilus (Bca) DNAP, Sulfolobus acidocaldarius (Sac) DNAP, Thermoplasma acidophilum (Tac) DNAP, Thermus flavus (Tfl/Tub) DNAP, Thermus ruber (Tru) DNAP, Thermus brockianus (DYNAZYMET™) DNAP, Thermosipho africanus DNAP, Thermococcus zilligi (Tzi) and mutants, variants and derivatives thereof (see e.g., U.S. Pat. No. 6,077,664; U.S. Pat. No. 5,436,149; U.S. Pat. No. 4,889,818; U.S. Pat. No. 5,532,600; U.S. Pat. No. 4,965,188; U.S. Pat. No. 5,079,352; U.S. Pat. No. 5,614,365; U.S. Pat. No. 5,374,553; U.S. Pat. No. 5,270,179; U.S. Pat. No. 5,047,342; U.S. Pat. No. 5,512,462; WO 94/26766; WO 92/06188; WO 92/03556; WO 89/06691; WO 91/09950; 91/09944; WO 92/06200; WO 96/10640; WO 97/09451; PCT WO 03/025132; U.S. Provisional Patent Application Ser. No. 60/647,408, filed Jan. 28, 2005; Barnes, W. Gene 112:29-35 (1992); Lawyer, F. et al. (1993) PCR Meth. Appl. 2:275-287; and Flaman, J. et al. (1994) Nucl. Acids Res. 22:3259-3260). Other DNAPs are mesophilic, including pol I family DNAPs (e.g., DNAPs from E. coli, H. influenzae, D. radiodurans, H. pylori, C. aurantiacus, R. Prowazekii, T. pallidum, Synechocysis sp., B. subtilis, L. lactis, S. pneumoniae, M tuberculosis, M leprae, M smegmatis, Bacteriophage L5, phi-C31, T7, T3, T5, SP01, SP02, S. cerevisiae, and D. melanogaster), pol III type DNAPs, and mutants, variants and derivatives thereof.

RNA-dependent DNA polymerases (reverse transcriptases) are enzymes having reverse transcriptase activity (i.e., that catalyze synthesis of DNA from a single-stranded RNA template). Such enzymes include, but are not limited to, retroviral reverse transcriptase, retrotransposon reverse transcriptase, hepatitis B reverse transcriptase, cauliflower mosaic virus reverse transcriptase, bacterial reverse transcriptase, Tth DNA polymerase, Taq DNA polymerase (Saiki, R. K., et al. (1988) Science 239:487-491; U.S. Pat. Nos. 4,889,818 and 4,965,188), Tne DNA polymerase (WO 96/10640 and WO 97/09451), Tma DNA polymerase (U.S. Pat. No. 5,374,553) and mutants, variants or derivatives thereof (see e.g., WO 97/09451 and WO 98/47912). Some RTs have reduced, substantially reduced, or eliminated RNase H activity. By an enzyme “substantially reduced in RNase H activity” is meant that the enzyme has less than about 20%, more preferably less than about 15%, 10% or 5%, and most preferably less than about 2%, of the RNase H activity of the corresponding wild type or RNase H+ enzyme such as wild type Moloney Murine Leukemia Virus (M-MLV), Avian Myeloblastosis Virus (AMV) or Rous Sarcoma Virus (RSV) reverse transcriptases. The RNase H activity of any enzyme may be determined by a variety of assays, such as those described, for example, in U.S. Pat. No. 5,244,797, in Kotewicz, M. L., et al. (1988) Nucl. Acids Res. 16:265 and in Gerard, G. F., et al. (1992) FOCUS 14:91. Particularly preferred polypeptides for use in the invention include, but are not limited to, M-MLV H-reverse transcriptase, RSV H-reverse transcriptase, AMV H-reverse transcriptase, RAV (rous-associated virus) H-reverse transcriptase, MAV (myeloblastosis-associated virus) H-reverse transcriptase and HIV H-reverse transcriptase (see U.S. Pat. No. 5,244,797 and WO 98/47912). It will be understood by one of skill in the art that any enzyme capable of producing a DNA molecule from a ribonucleic acid molecule (i.e., having reverse transcriptase activity) may be equivalently used in the compositions, methods and kits of the invention.

Nucleic Acid Binding Domain: The nucleic acid-binding domain comprises a polypeptide domain capable of binding a nucleic acid template. The nucleic acid-binding domain may be structured to bind DNA, RNA, or DNA and RNA. The nucleic acid-binding domain preferably includes at least one known or putative RNA binding motif, one known or putative DNA binding motif, or at least one known or putative RNA binding motif and at least one known or putative DNA binding motif. The nucleic acid binding domain preferably embodies a oligonucleotide/oligosaccharide binding (OB) fold, with the RNA binding motifs and/or DNA binding motifs on defined portions of the fold (see below). Exemplary RNA binding motifs include polypeptide sequences GYGFI (see SEQ ID NOS: 26, 28, 30, 46, 66, 68, 70, and 72), VFVHW (see SEQ ID NOS: 26, 46, 66, 68, 70, and 72), and VFVHF (see SEQ ID NOS: 28 and 30). Exemplary DNA binding motifs include polypeptide sequences AIEM (see SEQ ID NOS: 26, 46, 66, and 68), AIQG (see SEQ ID NO: 28), AIQN (see SEQ ID NO: 30), VGKM (see SEQ ID NOS: 32 and 52), VGKA (see SEQ ID NOS: 34, 44, 48, 50, 54, 56, 58, 68, 70, and 72), AGKA (see SEQ ID NOS: 36 and 42), and LAPKGRKGVKI (see SEQ ID NO: 38). As used herein, “DNA-binding motif” includes the DNA-binding loops between the β3 and β4 beta sheets on the OB folds. The nucleic acid binding domain may be thermostable.

The OB-fold domains, RNA-binding motifs, and/or DNA binding motifs contained on the OB-fold domains may be derived from Thermotoga maritime Cold shock protein (TmCsp; SEQ ID NO: 26); Bacillus caldolyticus Cold shock protein (BcCsp; SEQ ID NO: 28); E. coli Cold shock protein (EcCsp SEQ ID NO: 30); Archaeal basic protein from Sulfolobus solfataricus (Sso7d; SEQ ID NO: 32); Sulfolobus acidocaldarius engineered nucleic acid-binding protein (Sac7d mutant VA; SEQ ID NO: 34); Sulfolobus acidocaldarius engineered nucleic acid-binding protein (Sac7d mutant AA; SEQ ID NO: 36); Sulfolobus shibatae crenarchaeal 7K protein (SshCren7; SEQ ID NO: 38); Thermus brockianus single-stranded DNA-binding protein (Tbr SSB; SEQ ID NO: 40); and combinations thereof. See FIGS. 1A, 2A-D, 3A, and 4A-B.

A preferred version includes chimeric 08-fold domains, i.e., proteins comprising sequences from more than one 08-fold proteins described herein. Thus, for example, an RNA-binding motif and/or a DNA-binding motif from a first OB-fold protein, such as TmCsp, may replace sequences of a second OB-fold protein, such as Sac7d mutant VA, wherein the OB-fold is maintained in the second OB-fold protein and the RNA- and/or DNA-binding motifs are contained within the OB-fold of the second protein in an analogous position as in the OB-fold of the first protein. Various motifs from any OB-fold protein may replace sequences in any other OB-fold protein, as long as the OB-fold three-dimensional structure is maintained and the nucleic acid-binding activity is maintained. An exemplary version of such a chimeric protein is SEQ. ID NO: 70, which replaces sequences comprising the β3 beta sheet and the β4 beta sheet of the Sac7d mutant VA with the RNP-1 and RNP-2 binding motifs from TmCsp. See FIGS. 4A, 4B, and 4C. A full fusion protein containing the chimeric domain is SEQ ID NO: 72.

In an alternative version, the nucleic acid-binding domain may comprise a non-OB-fold protein that binds DNA and/or RNA. Such proteins preferably bind DNA and/or RNA in a non-sequence-specific manner. Preferred examples of RNA-binding proteins include avian myeloblastosis virus p12 basic protein (Smith and Bailey, 1979; Sykora and Moelling, 1981), HIV p7 nucleocapsid protein (Herschlag et al., 1994), and brine shrimp artemin (Chen et al., 2003).

Homologs and Variants: The invention further includes variants and homologs of the polypeptides herein (and nucleotides encoding them), including the polymerase domains, nucleic acid-binding domains, and full fusion proteins.

Homologs and variants suitable for the compositions and methods of the invention can be identified by homologous nucleotide and polypeptide sequence analyses. Known polypeptides in one organism can be used to identify homologous polypeptides in another organism. For example, performing a query on a database of nucleotide or polypeptide sequences can identify homologs of a known polypeptide. Homologous sequence analysis can involve BLAST or PSI-BLAST analysis of databases using known polypeptide amino acid sequences. Those proteins in the database that have greater than 35% sequence identity are candidates for further evaluation for suitability in the compositions and methods of the invention. If desired, manual inspection of such candidates can be carried out in order to narrow the number of candidates that can be further evaluated. Manual inspection is performed by selecting those candidates that appear to have domains conserved among known polypeptides.

The variants may comprise conservative substitutions of amino acids in the sequences described herein. A “conservative substitution” means the replacement of one amino acid by an amino acid having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The variant polypeptides include amino acid sequences with about 50%, 60%, 70%, 75%, 80%, 85%, 90%, 95%, 96%, 97%, 98%, or more identity to the sequences described herein. The term “identity” and grammatical variations thereof, mean that two or more referenced entities are the same. Thus, where two protein sequences are identical, they have the same amino acid sequence. The extent of identity between two sequences can be ascertained using a computer program and mathematical algorithm known in the art. Such algorithms that calculate percent sequence identity (homology) generally account for sequence gaps and mismatches over the comparison region. For example, a BLAST (e.g., BLAST 2.0) search algorithm (see, e.g., Altschul et al., J. Mol. Biol. 215:403-10 (1990), publicly available through NCBI) has exemplary search parameters as follows: Mismatch-2; gap open 5; gap extension 2. For polypeptide sequence comparisons, a BLASTP algorithm is typically used in combination with a scoring matrix, such as PAM100, PAM 250, and BLOSUM 62.

The invention includes fragments of the polypeptides described herein and of the nucleic acids encoding them. “Fragment” means a portion of the full length molecule. For example, a fragment of a given polypeptide is at least one amino acid fewer in length than the full length polypeptide (e.g. one or more internal or terminal amino acid deletions from either amino or carboxy-termini). Fragments therefore can be any length up to, but not including, the full length polypeptide. Suitable fragments of the polypeptides described herein include but are not limited to those having 50%, 60%, 70%, 75%, 80%, 85%, 90%, 95%, 96%, 97%, 98%, or more of the length of the full length polypeptide.

The invention includes polypeptides having repeating units of the sequences described herein. “Repeating units” means a repetition of a given sequence in tandem. Also included are polypeptides having repeating units of fragments of the sequences described herein.

Suitable variants, homologs, fragments, and repeating units of the polypeptides disclosed herein have DNA-binding activity and polymerase activity. Such activities may be tested according to the assays described in the Examples below.

OB-Fold RNA-Binding Proteins: Exemplary OB-fold RNA-binding proteins include cold shock proteins (Csps). Csps, originally discovered in E. coli (Jiang et al., 1997) and B. subtilus (Graumann et al., 1997; Weber and Mahariel, 2002), are small OB-fold proteins that are abundantly produced by bacteria in response to growth at low temperatures.

Cold shock proteins are found in all prokaryotes, except for the archaea and cyanobacteria (Weber and Mahariel, 2002). Csps facilitate unwinding of RNA secondary structure and facilitate mRNA translation at suboptimal growth temperatures. RNA-binding is mediated by the conserved RNA-binding motifs RNP-1 and RNP-2 (Bandzulis et al., 1989; Landsman, 1992; FIGS. 1A, 2A, 2D, and 3A). Due to their ability to bind non-specifically to RNA and to destabilize RNA hairpins, Csps have been referred to as “RNA chaperones” (Phadtare and Inouye, 1999).

Csps share limited (˜20%) amino acid sequence identity with archaeal Sso7, Sac7d and Cren7 proteins, but their mechanism of nucleic acid-binding is quite different (Feng et al., 1998). Sso/Sac7d proteins are arranged as 5-stranded antiparallel β-barrels (OB-folds). Hydrophobic residues in the flexible loop between beta sheets β3 and β4 contact the DNA minor groove (Kerr et al., 2003; Wang et al., 2004; Chen et al., 2005). Csps are also 5-stranded OB-fold proteins, but RNA-binding is mediated by RNP-1 and RNP-2 motifs located in beta sheets β2 and β3 (Phadtare and Inouye, 1999; Wang et al., 2000; FIGS. 1A, 2A, 2D, and 3A).

Three cold shock proteins have been subjected to detailed NMR and/or X-ray crystallographic structural analysis: EcCspA from E. coli (Schindelin et al., 1994; Newkirk et al., 1994), BcCsp from Bacillus caldolyticus (Mueller et al., 2000), and TmCsp from Thermotoga maritima (Jung et al., 2004). Two of these well-characterized Csps are thermostable: BcCsp and TmCsp.

The Thermotoga maritima cold shock protein (TmCsp; Welker et al., 1999; Phadtare et al., 2003) binds non-specifically to RNA. TmCsp is able to “melt” RNA secondary structure at temperatures as high as 70° C., displays a thermal denaturation temperature midpoint of 87° C. (Phadtare et al., 2003), and rapidly renatures to form a 5-stranded β-sheet OB-fold structure after thermal denaturation.

The invention includes other known RNA-binding OB-fold proteins or those that may be discovered.

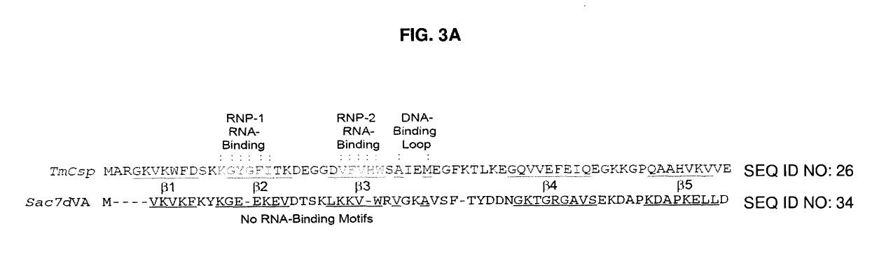

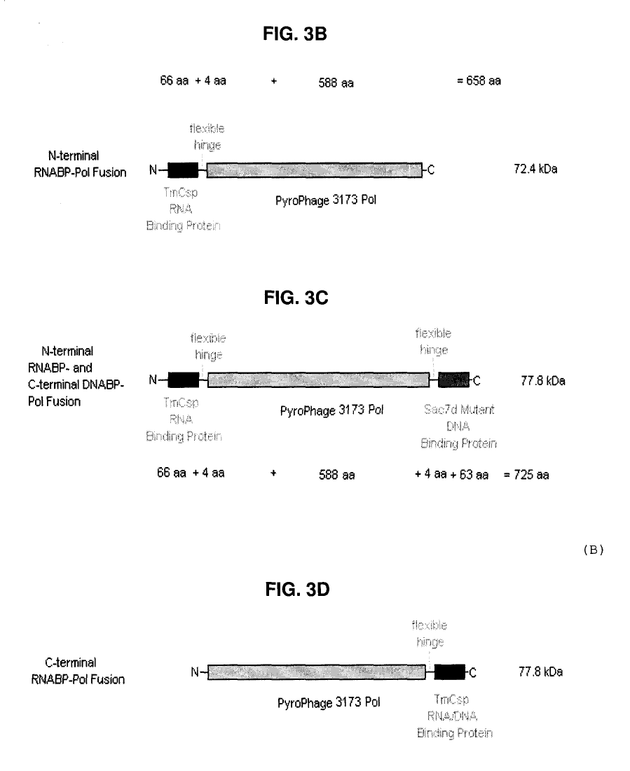

OB-Fold DNA-Binding Proteins: Exemplary OB-fold DNA-binding proteins include archaeal dsDNA-binding proteins and proteins related thereto. Small (60-70 amino acid), basic DNA-binding proteins from archaea, such as Sso7d and Sac7d assist replication in vivo by stabilizing double-stranded DNA at elevated temperatures (Grote et al., 1986). These archaeal DNA-binding proteins, and distantly related ˜60 amino acid DNA-binding proteins from Crenarchaeota (Cren7 proteins; Guo et al., 2008), share the OB-fold 5-stranded antiparallel β-sheet architecture (Murzin, 1993). Nuclear magnetic resonance and X-ray crystal structural analyses indicate that hydrophobic residues in the flexible loop connecting beta sheets β3 and β4 contact the DNA minor groove (Baumann et al., 1994; Newkirk et al., 1994; Feng et al., 1998; Kerr et al., 2003; Theobald et al., 2003, Chen et al., 2005; FIGS. 2A, 2B, 2C, and 3A).

Other exemplary DNA-binding OB-proteins include single stranded DNA binding proteins (SSBs). SSBs are proteins that preferentially bind single stranded DNA (ssDNA) over double-stranded DNA (dsDNA) in a nucleotide sequence independent manner. SSBs have been identified in virtually all known organisms, and appear to be important for DNA metabolism, including replication, recombination and repair. Naturally occurring SSBs typically are comprised of two, three or four subunits, which may be the same or different. In general, naturally occurring SSB subunits contains at least one conserved DNA binding domain within the “OB fold” (see e.g., Philipova, D. et al. (1996) Genes Dev. 10:2222-2233; and Murzin, A. (1993) EMBO J. 12:861-867). Naturally occurring SSBs may have four or more OB folds.

Thermostable SSBs bind ssDNA at 70° C. at least 70% (e.g., at least 80%, at least 85%, at least 90% and at least 95%) as well as they do at 37° C., and are better suited for PCR applications than are mesophilic SSBs. Thermostable SSBs can be obtained from archaea. Archaea are a group of microbes distinguished from eubacteria through 16S rDNA sequence analysis. Archaea can be subdivided into three groups: crenarchaeota, euryarchaeota and korarchaeota (see e.g., Woese, C. and G. Fox (1977) PNAS 74: 5088-5090; Woese, C. et al. (1990) PNAS 87: 4576-4579; and Barns, S. et al. (1996) PNAS 93:9188-9193). Recently, there have been reports on the identification and characterization of euryarchaeota SSBs, including Methanococcus jannachii SSB, Methanobacterium thermoautrophicum SSB, and Archaeoglobus fulgidus SSB, as well as crenarchaeota SSBs, including Sulfolobus solfataricus SSB and Aeropyrum pernix SSB (see e.g., Chedin, F. et al. (1998) Trends Biochem. Sci. 23:273-277; Haseltine C. et al. (2002) Mol. Microbiol. 43:1505-1515; Kelly, T. et al. (1998) Proc. Natl. Acad. Sci. USA 95:14634-14639; Klenk, H. et al. (1997) Nature 390:364-370; Smith, D. et al. (1997) J. Bacteriol. 179:7135-55; Wadsworth, R. and M. White (2001) Nucl. Acids Res. 29:914-920; and in U.S. Patent Application 60/147,680.

The invention includes other known DNA-binding OB-fold proteins or those that have yet to be discovered.

Nucleic Acid: In general, a nucleic acid comprises a contiguous series (a.k.a., “strand” and “sequence”) of nucleotides joined by phosphodiester bonds. A nucleic acid can be single stranded or double stranded, where two strands are linked via noncovalent interactions between complementary nucleotide bases. A nucleic acid can include naturally occurring nucleotides and/or non-naturally occurring base moieties. A nucleic acid can be ribonucleic acid (RNA, including mRNA) or deoxyribonucleic acid (DNA, including genomic DNA, recombinant DNA, cDNA and synthetic DNA). A nucleic acid can be a discrete molecule such as a chromosome or cDNA molecule. A nucleic acid can also be a segment (i.e. a series of nucleotides connected by phosphodiester bonds) of a discrete molecule.

Template: A template is a single stranded nucleic acid that, when part of a primer-template complex, can serve as a substrate for a polymerase. The template can be DNA (for DNA-dependent DNA polymerase) or RNA (for RNA-dependent DNA polymerase). A nucleic acid synthesis mixture can include a single type of template, or can include templates having different nucleotide sequences. By using primers specific for particular templates, primer extension products can be made for a plurality of templates in a nucleic acid synthesis mixture. The plurality of templates can be present within different discrete nucleic acids, or can be present within a discrete nucleic acid.

Templates can be obtained, or can be prepared from nucleic acids present in biological sources. (e.g. cells, tissues, body fluids, organs and organisms). Thus, templates can be obtained, or can be prepared from nucleic acids present in bacteria (e.g. species of Escherichia, Bacillus, Serratia, Salmonella, Staphylococcus, Streptococcus, Clostridium, Chlamydia, Neisseria, Treponema, Mycoplasma, Borrelia, Legionella, Pseudomonas, Mycobacterium, Helicobacter, erwinia, Agrobacterium, Rhizobium and Streptomyces), fungi such as yeasts, viruses (e.g., Orthomyxoviridae, Paramyxoviridae, Herpesviridae, Picornaviridae, Hepadnaviridae, Retroviridae), protozoa, plants and animals (e.g., insects such as Drosophila app., nematodes such as C. elegans, fish, birds, rodents, porcines, equines, felines, canines and primates, including humans. Templates can also be obtained, or can be prepared from, nucleic acids present in environmental samples such as soil, water and air samples. Nucleic acids can be prepared from such biological and environmental sources using routine methods known by those of skill in the art.

In some embodiments, a template is obtained directly from a biological or environmental source. In other embodiments, a template is provided by wholly or partially denaturing a double-stranded nucleic acid obtained from a biological or environmental source. In some embodiments, a template is a recombinant or synthetic DNA molecule. Recombinant or synthetic DNA can be single stranded or double stranded. If double stranded, the template may be wholly or partially denatured to provide a template. In some embodiments, the template is an mRNA molecule or population of mRNA molecules. In other embodiments, the template is a cDNA molecule of a population of cDNA molecules. A cDNA template can be synthesized in a nucleic acid synthesis reaction by an enzyme having reverse transcriptase activity, or can be provided from an extrinsic source (e.g., a cDNA library).

Primer: A primer is a single stranded nucleic acid that is shorter than a template, and is complementary to a segment of a template. A primer can hybridize to a template to form a primer-template complex (i.e., a primed template) such that a DNAP can synthesize a nucleic acid molecule (i.e., primer extension product) that is complementary to all or a portion of a template.

Primers typically are 12 to 60 nucleotides long (e.g. 18 to 45 nucleotides long), although they may be shorter or longer in length. A primer is designed to be substantially complementary to a cognate template such that it can specifically hybridize to the template to form a primer-template complex that can serve as a substrate for a polymerase to make a primer extension product. In some primer-template complexes, the primer and template are exactly complementary such that each nucleotide of a primer is complementary to and interacts with a template nucleotide. Primers can be made by methods well known in the art (e.g., using an ABI DNA Synthesizer from Applied Biosystems or a Biosearch 8600 or 8800 Series Synthesizer from Milligen-Biosearch, Inc.), or can be obtained from a number of commercial vendors.

Nucleotide: A nucleotide consists of a phosphate group linked by a phosphoester bond to a pentose (ribose in RNA, and deoxyribose in DNA) that is linked in turn to an organic base. The monomeric units of a nucleic acid are nucleotides. Naturally occurring DNA and RNA each contain four different nucleotides: nucleotides having adenine, guanine, cytosine and thymine bases are found in naturally occurring DNA, and nucleotides having adenine, guanine, cytosine and uracil bases found in naturally occurring RNA. The bases adenine, guanine, cytosine, thymine, and uracil often are abbreviated A, G, C, T and U, respectively.

Nucleotides include free mono-, di- and triphosphate forms (i.e., where the phosphate group has one, two or three phosphate moieties, respectively). Thus, nucleotides include ribonucleoside triphosphates (e.g., ATP, UTP, CTG and GTP) and deoxyribonucleoside triphosphates (e.g., dATP, dCTP, dITP, dGTP and dTTP), and derivatives thereof. Nucleotides also include dideoxyribonucleoside triphosphates (ddNTPs, including ddATP, ddCTP, ddGTP, ddITP and ddTTP), and derivatives thereof.

Nucleotide derivatives include [αS]dATP, 7-deaza-dGTP, 7-deaza-dATP, and nucleotide derivatives that confer resistance to nucleolytic degradation. Nucleotide derivatives include nucleotides that are detectably labeled, e.g., with a radioactive isotope such as ³²P or ³⁵S, a fluorescent moiety, a chemiluminescent moiety, a bioluminescent moiety, or an enzyme.

Primer Extension Product: A primer extension product is a nucleic acid that includes a primer to which polymerase has added one or more nucleotides. Primer extension products can be as long as, or shorter than the template of a primer-template complex.

Amplifying: Amplifying refers to an in vitro method for increasing the number of copies of a nucleic acid with the use of a polymerase. Nucleic acid amplification results in the addition of nucleotides to a primer or growing primer extension product to form a new molecule complementary to a template. In nucleic acid amplification, a primer extension product and its template can be denatured and used as templates to synthesize additional nucleic acid molecules. An amplification reaction can consist of many rounds of replication (e.g., one PCR may consist of 5 to 100 “cycles” of denaturation and primer extension). General methods for amplifying nucleic acids are well-known to those of skill in the art (see e.g., U.S. Pat. Nos. 4,683,195; 4,683,202; and 4,800,159; Innis, M. A., et al., eds., PCR Protocols: A Guide to Methods and Applications, San Diego, Calif.: Academic Press, Inc. (1990); Griffin, H., and A. Griffin, eds., PCR Technology: Current Innovations, Boca Raton, Fla.: CRC Press (1994)). Amplification methods that can be used in accord with the present invention include PCR (U.S. Pat. Nos. 4,683,195 and 4,683,202), Strand Displacement Amplification (SDA; U.S. Pat. No. 5,455,166; EP 0 684 315), Nucleic Acid Sequenced-Based Amplification (NASBA; U.S. Pat. No. 5,409,818; EP 0 329 822), among others.

Isolated: With respect to polypeptides, “isolated” refers to a polypeptide that constitutes a major component in a mixture of components, e.g., 30% or more, 40% or more, 50% or more, 60% or more, 70% or more, 80% or more, 90% or more, or 95% or more by weight. Isolated polypeptides typically are obtained by purification from an organism that contains the polypeptide (e.g., a transgenic organism that expresses the polypeptide), although chemical synthesis is also feasible. Methods of polypeptide purification include, for example, ammonium sulfate precipitation, chromatography and immunoaffinity techniques.

A polypeptide of the invention can be detected by any means known in the art, including sodium dodecyl sulfate (SDS)-polyacrylamide gel electrophoresis followed by Coomassie Blue-staining or Western blot analysis using monoclonal or polyclonal antibodies that have binding affinity for the polypeptide to be detected.

Thermostable: “Thermostable” refers to an enzyme or protein (e.g., polymerases and nucleic acid-binding proteins) that is resistant to inactivation by heat. In general, a thermostable protein is more resistant to heat inactivation than a mesophilic protein. Thus, the nucleic acid synthesis activity or single stranded binding activity of thermostable enzyme or protein may be reduced by heat treatment to some extent, but not as much as mesophilic enzyme or protein.

A thermostable protein retains at least 50% (e.g., at least 60%, at least 70%, at least 80%, at least 90%, and at least 95%) of its nucleic acid synthetic or binding activity after being heated in a nucleic acid synthesis mixture at 90° C. for 30 seconds. In contrast, mesophilic proteins lose most of their nucleic acid synthetic or binding activity after such heat treatment. Thermostable proteins typically also have a higher optimum nucleic acid synthesis or binding temperature than the mesophilic proteins.

The degree to which an OB-fold nucleic acid-binding protein binds DNA at such temperatures can be determined by measuring intrinsic protein fluorescence. Intrinsic protein fluorescence is related to conserved OB fold amino acids, and is quenched upon binding to DNA (see e.g., Alani, E. et al. (1992) J. Mol. Biol. 227:54-71). A routine protocol for determining DNA binding is described in Kelly, T. et al. (1998) Proc. Natl. Acad. Sci. USA 95:14634-14639. Briefly, DNA binding reactions are performed in 2 ml buffer containing 30 mM HEPES (pH 7.8), 100 mM NaCl, 5 mM MgCl2, 0.5% inositol and 1 mM DTT. A fixed amount of the nucleic acid-binding protein is incubated with varying quantities of poly(dT), and fluorescence is measured using an excitation wavelength of about 295 nm and an emission wavelength of about 348 nm.

Vector: A vector is a nucleic acid such as a plasmid, cosmid, phage, or phagemid that can replicate autonomously in a host cell. A vector has one or a small number of sites that can be cut by a restriction endonuclease in a determinable fashion, and into which DNA can be inserted. A vector also can include a marker suitable for use in identifying hosts that contain the vector. Markers confer a recognizable phenotype on host cells in which such markers are expressed. Commonly used markers include antibiotic resistance genes such as those that confer tetracycline resistance or ampicillin resistance. Vectors also can contain sequences encoding polypeptides that facilitate the introduction of the vector into a host. Such polypeptides also can facilitate the maintenance of the vector in a host. “Expression vectors” include nucleic acid sequences that can enhance and/or regulate the expression of inserted DNA, after introduction into a host. Expression vectors contain one or more regulatory elements operably linked to a DNA insert. Such regulatory elements include promoter sequences, enhancer sequences, response elements, protein recognition sites, or inducible elements that modulate expression of a nucleic acid. As used in this context, “operably linked” refers to positioning of a regulatory element in a vector relative to a DNA insert in such a way as to permit or facilitate transcription of the insert and/or translation of resultant RNA transcripts. The choice of element(s) included in an expression vector depends upon several factors, including, replication efficiency, selectability, inducibility, desired expression level, and cell or tissue specificity.

DNA sequences encoding the nucleic acid-binding proteins, polymerases, and fusion proteins described herein include: SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, and 71.

Host: The term “host” includes prokaryotes, such as E. coli, and eukaryotes, such as fungal, insect, plant and animal cells. Animal cells include, for example, COS cells and HeLa cells. Fungal cells include yeast cells, such as Saccharomyces cereviseae cells. A host cell can be transformed or transfected with a vector using techniques known to those of ordinary skill in the art, such as calcium phosphate or lithium acetate precipitation, electroporation, lipofection and particle bombardment. Host cells that contain a vector or portion thereof (a.k.a. “recombinant hosts”) can be used for such purposes as propagating the vector, producing a nucleic acid (e.g., DNA, RNA, antisense RNA) or expressing a polypeptide. In some cases, a recombinant host contains all or part of a vector (e.g., a DNA insert) on the host genome.

Expression and Purification of Fusion Proteins: To optimize expression of the fusion proteins described herein, inducible or constitutive promoters well known in the art may be used to control expression of a recombinant fusion protein gene in a recombinant host. Similarly, high or low copy number vectors, well known in the art, may be used to achieve appropriate levels of expression. Vectors having an inducible high copy number may also be useful to enhance expression of the fusion proteins in a recombinant host.

Prokaryotic vectors for constructing the plasmid library include plasmids such as those capable of replication in E. Coli, including, but not limited to, pBR322, pET-26b(+), ColE1, pSC101, pUC vectors (pUC18, pUC19, etc., in Molecular Cloning, a Laboratory Manual, Cold Spring Harbor Laboratory Press, Cold Spring Harbor, N.Y., 1989). Bacillus plasmids include pC194, pC221, pC217, etc. (Glyczan, in Molecular Biology Bacilli, Academic Press, New York, pp 307-329. 1982). Suitable Streptomyces plasmids include pIJ101 (Kendall et al., J. Bacteriol. 169:4177-4183, 1987). Pseudomonas plasmids are reviewed by John et al. (Rad. Insec. Dis. 8:693-704, 1986) and Igaki (Jpn. J. Bacteriol. 33:729-742, 1978). Broad-host range plasmids or cosmids, such as pCP13 (Darzins et al., J. Bacteriol. 159:9-18, 1984) can also be used.

The fusion protein may be cloned in a prokaryotic host such as E. coli or other bacterial species including, but not limited to, Escherichia, Pseudomonas, Salmonella, Serratia, and Proteus. Eukaryotic hosts also can be used for cloning and expression of wild type or mutant polymerases. Such hosts include yeast, fungi, insect and mammalian cells. Expression of the desired DNA polymerase in such eukaryotic cells may involve the use of eukaryotic regulatory regions which include eukaryotic promoters. Cloning and expressing the fusion proteins in eukaryotic cells may be accomplished by well known techniques using well known eukaryotic vector systems.

Hosts can be transformed by routine, well-known techniques. In one embodiment, transformed colonies are plated and screened for the expression of a fusion protein by transferring transformed E. coli colonies to nitrocellulose membranes. After the transformed cells are grown on nitrocellulose, the cells are lysed by standard techniques, and the membranes are then treated at 95° C. for 5 minutes to inactivate the endogenous E. coli enzyme. Other temperatures may be used to inactivate the host polymerases depending on the host used and the temperature stability of the fusion protein to be cloned. Fusion protein activity is then detected by assaying for the presence of DNA polymerase activity using well known techniques (i.e. Sanger et al., Gene 97:119-123, 1991).

Also included in the invention are host cells that contain or comprise nucleic acid molecules, and vectors that contain or comprise these nucleic acid molecules. Other aspects include compositions and mixtures (e.g., reaction mixtures) that contain or comprise one or more polypeptides and/or more polynucleotides described herein.

To optimize expression of the fusion proteins, inducible or constitutive promoters are well known and may be used to express high levels of a fusion protein in a recombinant host. Similarly, high copy number vectors, well known in the art, may be used to achieve or enhance expression of the fusion protein in a recombinant host.

To express the desired fusion protein in a prokaryotic cell (such as, E. coli, B. subtilis, Pseudomonas, etc.), the gene encoding the fusion protein may be operably linked to a functional prokaryotic promoter. However, the natural promoter may function in prokaryotic hosts allowing expression of the fusion protein. Thus, the natural promoter or other promoters may be used to express the fusion protein. Such other promoters may be used to enhance expression and may either be constitutive or regulatable (i.e., inducible or derepressible) promoters. Examples of constitutive promoters include the int promoter of bacteriophage λ, and the bla promoter of the β-lactamase gene of pBR322. Examples of inducible prokaryotic promoters include the major right and left promoters of bacteriophage λ. (PR and PL), trp, recA, lacZ, lacI, tet, gal, trc, and tac promoters of E. coli. The B. subtilis promoters include α-amylase (Ulmanen et al., J. Bacteriol 162:176-182 (1985)) and Bacillus bacteriophage promoters (Gryczan, T., supra.). Streptomyces promoters are described by Ward et al., Mol. Gen. Genet. 203:468-478, 1986). Prokaryotic promoters are also reviewed by Glick, J. Ind. Microbiol. 1:277-282, 1987; Cenatiempto, Y., Biochimie 68:505-516, 1986; and Gottesman, Ann. Rev. Genet. 18:415-442 (1984). Expression in a prokaryotic cell also requires the presence of a ribosomal binding site upstream of the gene-encoding sequence. Such ribosomal binding sites are disclosed, for example, by Gold et al., Ann. Rev. Microbiol. 35:365-404 (1981).

In one embodiment, the fusion proteins described herein are produced by fermentation of the recombinant host containing and expressing the cloned fusion protein gene. Any nutrient that can be assimilated by the thermophile of interest, or a host containing the cloned fusion protein gene, may be added to the culture medium. Optimal culture conditions should be selected case by case according to the strain used and the composition of the culture medium. Antibiotics may also be added to the growth media to insure maintenance of vector DNA containing the desired gene to be expressed.

Recombinant host cells producing the fusion proteins of the invention can be separated from liquid culture, for example, by centrifugation. In general, the collected microbial cells are dispersed in a suitable buffer, and then broken down by ultrasonic treatment or by other well known procedures to allow extraction of the enzymes by the buffer solution. After removal of cell debris by ultracentrifugation or centrifugation, the fusion protein can be purified by standard protein purification techniques such as extraction, precipitation, chromatography, affinity chromatography, electrophoresis or the like. Assays to detect the presence of the fusion proteins during purification are well known in the art and can be used during conventional biochemical purification methods to determine the presence of these enzymes.

Use of Fusion Proteins: The fusion proteins described herein may be used in any application involving synthesizing a nucleic acid from a template. Examples, include DNA sequencing, DNA labeling, DNA amplification or cDNA synthesis reactions. The fusion proteins may also be used to analyze and/or type polymorphic DNA fragments

Nucleic Acid Synthesis: Fusion proteins may be used in nucleic acid synthesis reactions which comprise: (a) mixing one or more templates with one or more fusion proteins to form a mixture; and (b) incubating the mixture under conditions sufficient to make a nucleic acid complementary to all or a portion of the templates (i.e., a primer extension product). Reaction conditions sufficient to allow nucleic acid synthesis (e.g., pH, temperature, ionic strength, and incubation time) can be optimized according to routine methods known to those skilled in the art and may involve the use of one or more primers, one or more nucleotides, and/or one or more buffers or buffering salts, or any combination thereof.

Fusion proteins may be used in amplification methods comprising: (a) mixing one or more templates with one or more fusion proteins to form a mixture; and (b) incubating the mixture under conditions sufficient to amplify a nucleic acid complementary to all or a portion of the templates. Such conditions may involve the use of one or more primers, one or more nucleotides, one or more buffers and/or one or more buffering salts, or any combination thereof. Conditions to facilitate nucleic acid synthesis such as pH, ionic strength, temperature and incubation time can be determined as a matter of routine by those skilled in the art.

Following nucleic acid synthesis, nucleic acids can be isolated for further use or characterization. Synthesized nucleic acids can be separated from other nucleic acids and other constituents present in a nucleic acid synthesis reaction by any means known in the art, including gel electrophoresis, capillary electrophoresis, chromatography (e.g., size, affinity and immunochromatography), density gradient centrifugation, and immunoadsorption. Separating nucleic acids by gel electrophoresis provides a rapid and reproducible means of separating nucleic acids, and permits direct, simultaneous comparison of nucleic acids present in the same or different samples. Nucleic acids made by the provided methods can be isolated using routine methods. For example, nucleic acids can be removed from an electrophoresis gel by electroelution or physical excision. Isolated nucleic acids can be inserted into vectors, including expression vectors, suitable for transfecting or transforming prokaryotic or eukaryotic cells.

DNA Sequencing: Fusion proteins can be used in sequencing reactions (isothermal DNA sequencing and cycle sequencing of DNA). For example, fusion proteins can be used for dideoxy-mediated sequencing involves the use of a chain-termination technique which uses a specific polymer for extension by DNA polymerase, a base-specific chain terminator and the use of polyacrylamide gels to separate the newly synthesized chain-terminated DNA molecules by size so that at least a part of the nucleotide sequence of the original DNA molecule can be determined. Specifically, a DNA molecule is sequenced by using four separate DNA sequence reactions, each of which contains different base-specific terminators. For example, the first reaction will contain a G-specific terminator, the second reaction will contain a T-specific terminator, the third reaction will contain an A-specific terminator, and a fourth reaction may contain a C-specific terminator. Preferred terminator nucleotides include dideoxyribonucleoside triphosphates (ddNTPs) such as ddATP, ddTTP, ddGTP, ddITP and ddCTP. Analogs of dideoxyribonucleoside triphosphates may also be used and are well known in the art. Detectably labeled nucleotides are typically included in sequencing reactions. Any number of labeled nucleotides can be used in sequencing (or labeling) reactions, including, but not limited to, radioactive isotopes, fluorescent labels, chemiluminescent labels, bioluminescent labels, and enzyme labels.

The fusion proteins may also be used in cycle sequencing reactions. Cycle sequencing often involves the use of fluorescent dyes. In some cycle sequencing protocols, sequencing primers are labeled with fluorescent dye (e.g., using Amersham Bioscience MegaBACE DYEnamic ET Primers, ABI Prism® BigDye™ primer cycle sequencing kit, and Beckman Coulter WellRED fluorescence dye). Sequencing reactions using fluorescent primers offers advantages in accuracy and readable sequence length. However, separate reactions must be prepared for each nucleotide base for which sequence position is to be determined. In other cycle sequencing protocols, fluorescent dye is linked to ddNTP as a dye terminator (e.g., using Amersham Bioscience MegaBACE DYEnamic ET Terminator cycle sequencing kit, ABI Prism® BigDye™ Terminator cycle sequencing kit, ABI Prism® dRhodamine Terminator cycle sequencing kit, LI-COR IRDye™ Terminator Mix, and CEQ Dye Terminator Cycle sequencing kit with Beckman Coulter WellRED dyes). Since dye terminators can be labeled with unique fluorescence dye for each base, sequencing can be done in a single reaction.

Thus, nucleic acids may be sequenced by: (a) mixing one or more templates to be sequenced with one or more fusion proteins (and optionally one or more nucleic acid synthesis terminating agents such as ddNTPs) to form a mixture; (b) incubating the mixture under conditions sufficient to synthesize a population of molecules complementary to all or a portion of the template to be sequenced; and (c) separating the population to determine the nucleotide sequence of all or a portion of the template to be sequenced.

Polymerase Chain Reaction (PCR): Polymerase chain reaction (PCR), a well known DNA amplification technique, is a process by which DNA polymerase and deoxyribonucleoside triphosphates are used to amplify a target DNA template. In such PCR reactions, two primers, one complementary to the 3′ termini (or near the 3′-termini) of the first strand of the DNA molecule to be amplified, and a second primer complementary to the 3′ termini (or near the 3′-termini) of the second strand of the DNA molecule to be amplified, are hybridized to their respective DNA strands. After hybridization, DNA polymerase, in the presence of deoxyribonucleoside triphosphates, allows the synthesis of a third DNA molecule complementary to the first strand and a fourth DNA molecule complementary to the second strand of the DNA molecule to be amplified. This synthesis results in two double stranded DNA molecules. Such double stranded DNA molecules may then be used as DNA templates for synthesis of additional DNA molecules by providing a DNA polymerase, primers, and deoxyribonucleoside triphosphates. As is well known, the additional synthesis is carried out by “cycling” the original reaction (with excess primers and deoxyribonucleoside triphosphates) allowing multiple denaturing and synthesis steps. Typically, denaturing of double stranded DNA molecules to form single stranded DNA templates is accomplished by high temperatures. The fusion proteins described herein include those which are heat stable, and thus will survive such thermal cycling during DNA amplification reactions. Thus, these fusion proteins are ideally suited for PCR reactions, particularly where high temperatures are used to denature the DNA molecules during amplification. The fusion proteins may be used in all PCR methods known to one of ordinary skill in the art, including end-point PCR, real-time qPCR (U.S. Pat. Nos. 6,569,627; 5,994,056; 5,210,015; 5,487,972; 5,804,375; 5,994,076, the contents of which are incorporated by reference in their entirety), allele specific amplification, linear PCR, one step reverse transcriptase (RT)-PCR, two step RT-PCR, mutagenic PCR, multiplex PCR and the PCR methods described in copending U.S. patent application Ser. No. 09/599,594, the contents of which are incorporated by reference in their entirety.

Preparation of cDNA: The fusion proteins (reverse transcriptase fusion enzymes) described herein may also be used to prepare cDNA from mRNA templates. See, for example, U.S. Pat. Nos. 5,405,776 and 5,244,797, the disclosures of which are incorporated herein by reference. Thus, the invention also relates to a method of preparing cDNA from mRNA, comprising (a) contacting mRNA with an oligo(dT) primer or other complementary primer to form a hybrid; and (b) contacting the hybrid formed in step (a) with a fusion protein of the invention and the four dNTPs, whereby a cDNA-RNA hybrid is obtained. If the reaction mixture is step (b) further comprises an appropriate oligonucleotide which is complementary to the cDNA being produced, it is also possible to obtain dsDNA following first strand synthesis. Thus, the invention is also directed to a method of preparing dsDNA with the fusion proteins described herein. Use of fusion proteins in RT-PCR for other applications is also included in this invention.

Another embodiment features compositions and reactions for nucleic acid synthesis, sequencing or amplification that include the fusion proteins of the invention. These mixtures include one or more fusion proteins, one or more dNTPs (dATP, dTTP, dGTP, dCTP), a nucleic acid template, an oligonucleotide primer, magnesium and buffer salts, and may also include other components (e.g., nonionic detergent). If sequencing reactions are performed, the reaction may also include one or more ddNTPs. The dNTPs or ddNTPs may be unlabeled or labeled with a fluorescent, chemiluminescent, bioluminescent, enzymatic or radioactive label. In some embodiments, compositions comprising one or more fusion proteins are formulated as described in PCT WO 98/06736, the entire contents of which are incorporated herein by reference.

In some embodiments, kits are provided (e.g., for use in carrying out the methods described herein). Such kits may include, in addition to one or more fusion proteins, one or more components selected from the group consisting of: one or more host cells (preferably competent to take up nucleic acid molecules), one or more nucleic acids (e.g., nucleic acid templates), one or more nucleotides, one or more nucleic acid primers, one or more vectors, one or more ligases, one or more topoisomerases, and one or more buffers or buffer salts.

High-Temperature RT: In a further preferred embodiment of the invention, a fusion protein is used to reverse transcribe RNA into cDNA at temperatures greater than 45° C. This preferred embodiment offers several advantages over currently available techniques.

Moloney Murine Leukemia Virus (MoMLV-RT) is inactive at temperatures above 45° C.; and Avian Myeloblastosis Virus (AMV-RT) is inactive at temperatures above 48° C. (Yasukawa et al., 2008). In contrast, 3173 Pol has reverse transcriptase activity at 45° C. to 70° C. (Tom Schoenfeld, Lucigen Corp.); and Tth Pol has RT activity at 60° C. in the presence of Mn++ (Myers and Gelfand, 1991). At temperatures above 45° C., RNA secondary structure is disrupted and the reaction rate of DNA polymerization is greater than enzymatic copying at lower temperatures (Mizuno et al., 1999). Therefore, the ability to reverse transcribe RNA at 45° to 75° C. allows RT-PCR under reaction conditions which minimize RNA secondary structure.

One-Tube, One-Enzyme RT-PCR: In a further preferred embodiment of the invention, a fusion protein is used for reverse transcription of RNA into cDNA, followed by PCR amplification (U.S. Pat. No. 4,965,188 to Mullis et al.). Since a single enzyme is used to catalyze two sequential reactions, the need to transfer the first RT reaction product to a second reaction for PCR amplification is obviated.

RT-Isothermal DNA Amplification: In a further preferred embodiment of the invention, a fusion protein (comprised of an RNA-binding domain and a reverse transcriptase domain), is used to (a) reverse transcribe RNA into cDNA, followed by (b) isothermal amplification of DNA, using methods known to those practiced in the art (Notomi et al., 2000; Gill and Ghaemi, 2008) such as loop amplification and rolling circle amplification.

Diagnostic Tests: The fusion proteins may be used in diagnostic tests. One version includes analyzing and typing polymorphic DNA fragments. The relationship between a first individual and a second individual may be determined by analyzing and typing a particular polymorphic DNA fragment, such as a minisatellite or microsatellite DNA sequence. In such a method, the amplified fragments for each individual are compared to determine similarities or dissimilarities. Such an analysis is accomplished, for example, by comparing the size of the amplified fragments from each individual, or by comparing the sequence of the amplified fragments from each individual. In another aspect of the invention, genetic identity can be determined. Such identity testing is important, for example, in paternity testing, forensic analysis, etc. In this aspect of the invention, a sample containing DNA is analyzed and compared to a sample from one or more individuals. In one such aspect of the invention, one sample of DNA may be derived from a first individual and another sample may be derived from a second individual whose relationship to the first individual is unknown; comparison of these samples from the first and second individuals by the methods of the invention may then facilitate a determination of the genetic identity or relationship between the first and second a individual. In a particularly preferred such aspect, the first DNA sample may be a known sample derived from a known individual and the second DNA sample may be an unknown sample derived, for example, from crime scene material. In an additional aspect of the invention, one sample of DNA may be derived from a first individual and another sample may be derived from a second individual who is related to the first individual; comparison of these samples from the first and second individuals by the methods of the invention may then facilitate a determination of the genetic kinship of the first and second individuals by allowing examination of the Mendelian inheritance, for example, of a polymorphic, minisatellite, microsatellite or STR DNA fragment.

In another diagnostic test, DNA fragments important as genetic markers for encoding a gene of interest can be identified and isolated. For example, by comparing samples from different sources, DNA fragments which may be important in causing diseases such as infectious diseases (of bacterial, fungal, parasitic or viral etiology), cancers or genetic diseases, can be identified and characterized. In this aspect of the invention a DNA sample from normal cells or tissue is compared to a DNA sample from diseased cells or tissue. Upon comparison according to the invention, one or more unique polymorphic fragments present in one DNA sample and not present in the other DNA sample can be identified and isolated. Identification of such unique polymorphic fragments allows for identification of sequences associated with, or involved in, causing the diseased state.

Gel electrophoresis is typically performed on agarose or polyacrylamide sequencing gels according to standard protocols using gels containing polyacrylamide at concentrations of 3-12% (e.g., 8%), and containing urea at a concentration of about 4-12M (e.g., 8M). Samples are loaded onto the gels, usually with samples containing amplified DNA fragments prepared from different sources of genomic DNA being loaded into adjacent lanes of the gel to facilitate subsequent comparison. Reference markers of known sizes may be used to facilitate the comparison of samples. Following electrophoretic separation, DNA fragments may be visualized and identified by a variety of techniques that are routine to those of ordinary skill in the art, such as autoradiography. One can then examine the autoradiographic films either for differences in polymorphic fragment patterns (“typing”) or for the presence of one or more unique bands in one lane of the gel (“identifying”); the presence of a band in one lane (corresponding to a single sample, cell or tissue type) that is not observed in other lanes indicates that the DNA fragment comprising that unique band is source-specific and thus a potential polymorphic DNA fragment.

Nucleic Acid Synthesis Compositions: Nucleic acid synthesis compositions can include one or more fusion proteins, one or more nucleotides, one or more primers, one or more buffers and/or one or more templates. In some embodiments, a nucleic acid synthesis reaction can include mRNA and a fusion protein having reverse transcriptase activity. These compositions can be used to improve the yield and/or homogeneity of primer extension products made during nucleic acid synthesis (e.g., cDNA synthesis, amplification and combined cDNA synthesis/amplification reactions).

Kits: The fusion proteins described herein are suited for the preparation of a kit. Kits comprising these fusion proteins may be used for detectably labeling DNA molecules, DNA sequencing, amplifying DNA molecules or cDNA synthesis by well known techniques, depending on the content of the kit. See U.S. Pat. Nos. 4,962,020, 5,173,411, 4,795,699, 5,498,523, 5,405,776 and 5,244,797, the disclosures of which are hereby incorporated by reference. Such kits may comprise a carrying means being compartmentalized to receive in close confinement one or more container means such as vials, test tubes and the like. Each of such container means comprises components or a mixture of components needed to perform DNA sequencing, DNA labeling, DNA amplification, or cDNA synthesis.

Such kits may include, in addition to one or more fusion proteins, one or more components selected from the group consisting of one or more host cells (preferably competent to take up nucleic acid molecules), one or more nucleic acids (e.g., nucleic acid templates), one or more nucleotides, one or more nucleic acid primers, one or more vectors, one or more ligases, one or more topoisomerases, and one or more buffers or buffer salts.

Kit constituents typically are provided, individually or collectively, in containers (e.g., vials, tubes, ampules, and bottles). Kits typically include packaging material, including instructions describing how the kit can be used for example to synthesize, amplify or sequence nucleic acids. A first container may, for example, comprise a substantially purified sample of each fusion protein. A second container may comprise one or a number of types of nucleotides needed to synthesize a DNA molecule complementary to DNA template. A third container may comprise one or a number of different types of dideoxynucleoside triphosphates. A fourth container may comprise pyrophosphatase. In addition to the above containers, additional containers may be included in the kit which comprise one or a number of DNA primers. A kit used for amplifying DNA will comprise, for example, a first container comprising a substantially pure fusion protein as described herein and one or a number of additional containers which comprise a single type of nucleotide or mixtures of nucleotides. Various primers may or may not be included in a kit for amplifying DNA. The various kit components need not be provided in separate containers, but may also be provided in various combinations in the same container. For example, the fusion protein and nucleotides may be provided in the same container, or the fusion protein and nucleotides may be provided in different containers.

Kits for cDNA synthesis comprise a first container containing a fusion protein, a second container containing the four dNTPs and the third container containing an oligo(dT) primer. See U.S. Pat. Nos. 5,405,776 and 5,244,797, the disclosures of which are incorporated herein by reference. Since the fusion proteins of the present invention are also capable of preparing dsDNA, a fourth container may contain an appropriate primer complementary to the first strand cDNA. Of course, it is also possible to combine one or more of these reagents in a single tube. When desired, the kit of the present invention may also include a container which comprises detectably labeled nucleotides which may be used during the synthesis or sequencing of a DNA molecule. One of a number of labels may be used to detect such nucleotides. Illustrative labels include, but are not limited to, radioactive isotopes, fluorescent labels, chemiluminescent labels, bioluminescent labels and enzyme labels.

Any embodiment or part thereof may be used with any other embodiment or part thereof. The elements described herein can be used in any combination whether explicitly described or not. All combinations of method or process steps as used herein can be performed in any order, unless otherwise specified or clearly implied to the contrary by the context in which the referenced combination is made. As used in this specification and the appended claims, the singular forms “a,” “an,” and “the” include plural referents unless the content clearly dictates otherwise. The term “or” is generally employed in its sense including “and/or” unless the content clearly dictates otherwise. Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, 5, 6, from 1 to 9, from 3.6 to 4.6, from 3.5 to 9.9, and so forth.

All publications, patents, patent applications, and references cited herein are expressly incorporated by reference to the same extent as if each individual publication or patent application was specifically and individually indicated by reference. In case of conflict between the present disclosure and the incorporated patents, publications, and references, the present disclosure should control.

The embodiments of the present invention can comprise, consist of, or consist essentially of the limitations described herein, as well as any additional or optional steps, ingredients, components, or limitations described herein or otherwise useful in biochemistry, enzymology and/or genetic engineering.

It is understood that the invention is not confined to the particular construction and arrangement of parts herein illustrated and described, but embraces such modified forms thereof as come within the scope of the claims.

### EXAMPLES

### Example 1

To determine if the nucleotide binding proteins described herein retain their ability to bind nucleic acids after being fused to a polymerase, a gel shift assay was performed with a nucleic acid-binding/polymerase fusion protein.

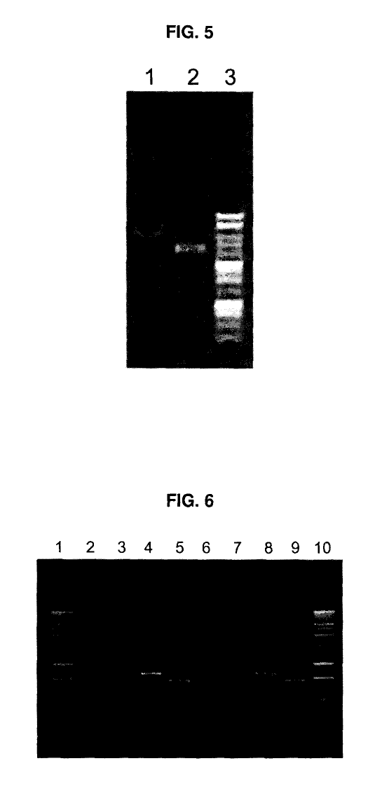

Bacteriophage M13 single stranded DNA (GenBank Acc. No. X02513) was incubated with (FIG. 5, lane 1) and without (FIG. 5, lane 2) a fusion protein comprising the SSB protein fused to PyroPhage 3173 DNA polymerase (SEQ ID NO: 62). As shown in FIG. 5, the mobility of the DNA shifted in the presence of the fusion protein (compare lanes 1 and 2), indicating that the fusion protein bound the DNA.

This example shows that the fusion proteins comprising a nucleic acid-binding domain and a polymerase domain bind DNA.

### Example 2

In this example, the ability of fusion proteins comprising a nucleic acid-binding domain and a polymerase domain in amplifying DNA through PCR was compared with that of a conventional DNA polymerase.

Human genomic DNA (gDNA) sequences were amplified with conventional Taq DNA polymerase (SEQ ID NO: 4) (FIG. 6, lanes 2, 3, 6, 7) or Taq Pol Δ289 (SEQ ID NO: 6) with the Sac 7d-V26/A29 protein (SEQ ID NO: 34) fused to its amino terminus (FIG. 6, lanes 4, 5, 8, 9). Human gDNA sequences were amplified with 5 micromolar each of 5′-AGATCCGCACGCACAACC-3′ (SEQ ID NO: 78) and 5′-CCTGCTCGCTCTCTCAATCTCT-3′ (SEQ ID NO: 79) (lanes 2, 4, 6, 8) or 5′-CTGGTCTGGCCCTGATGG-3′ (SEQ ID NO: 80) and 5′-CCTGGACGCCCTAACCTG-3′ (SEQ ID NO: 81) (lanes 3, 5, 7, 9) in 2% (lanes 2-5) or 4% blood (lanes 6-9). Reactions were performed in 1×“ECONO TAQ”-brand master mix (Lucigen, Madison, Wis.) cycled at 98° C. for 2 min and 40 cycles of 98° C. for 30 sec, 65° C. for 30 sec, and 72° C. for 45 sec. As shown in FIG. 6, the fusion protein was more effective in amplifying genomic DNA than the conventional Taq polymerase (compare lanes 4, 5, 8, and 9 with lanes 2, 3, 7, and 8).

This example shows that the fusion proteins comprising a nucleic acid-binding domain and a polymerase domain described herein are more effective than conventional polymerases in amplifying genomic DNA through PCR.

### Example 3

In this example, the ability of fusion proteins comprising a nucleic acid-binding domain and a polymerase domain in amplifying DNA in colony PCR was compared with that of a conventional DNA polymerase.

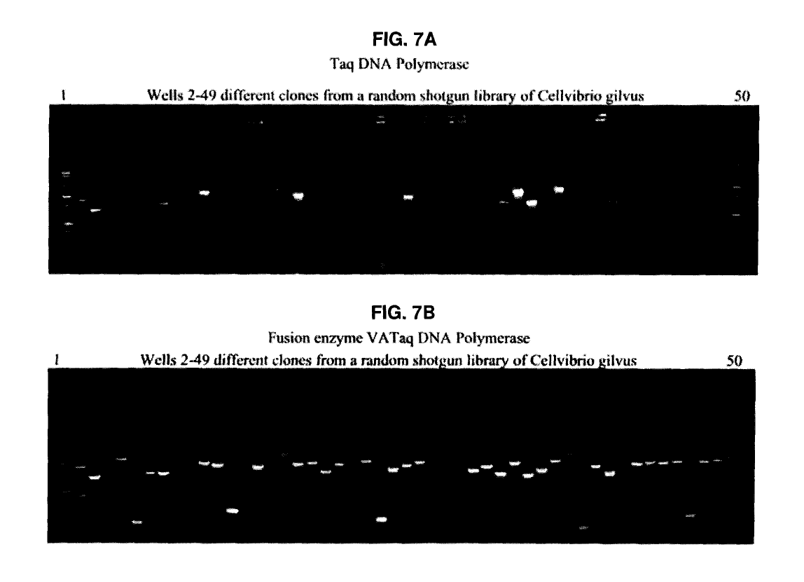

Random E. coli colonies approximately 0.5 mm in size were picked and resuspended into 40 μl 10 mM Tris pH 8.0. One microliter of the resuspended cells were amplified under identical conditions using two different polymerases: conventional Taq DNA polymerase (SEQ ID NO: 4) (FIG. 7A) or Taq Pol Δ289 (SEQ ID NO: 6) with the Sac7d-V26/A29 protein (SEQ ID 34) fused to its amino terminus (FIG. 7B). 12.5 microliter reactions were performed in 1× “ECONO TAQ”-brand master mix, cycled at 98° C. for 2 mM and 30 cycles of 98° C. for 30 sec, 65° C. for 15 sec, and 72° C. for 3 min using 0.5 uM of the following primers: 5′-TGAGCCAGTGAGTTGATTGCAGTCCA-3′ (SEQ ID NO: 73) and 5′-GAAGCGGGTTTTTACCTTATTTGCGG-3′ (SEQ ID NO: 74). As shown in FIGS. 7A and 7B, the fusion protein was more effective in amplifying DNA in colony PCR than the conventional Taq polymerase.

This example shows that the fusion proteins comprising a nucleic acid-binding domain and a polymerase domain are more effective than conventional polymerases in amplifying DNA in colony PCR.

### Example 4

In this example, polymerases fused to different nucleic acid binding proteins were compared for their ability to amplify DNA.

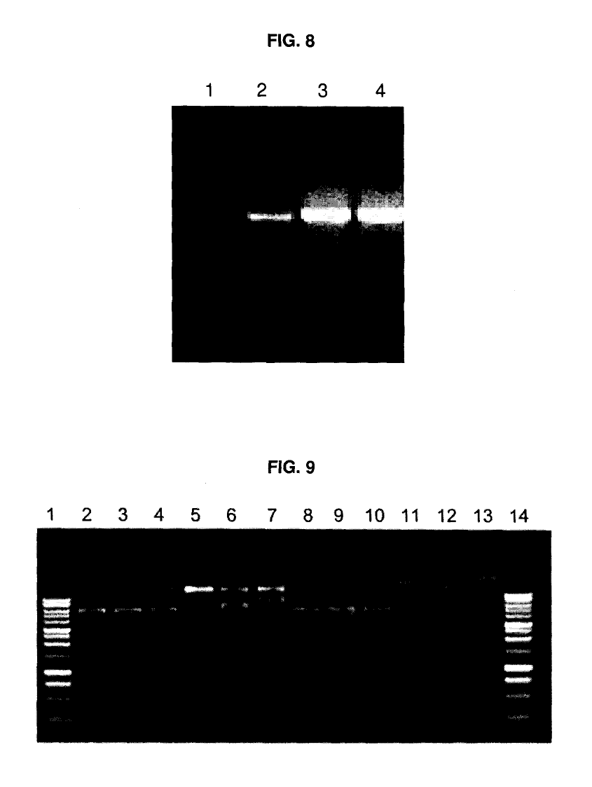

Primers were designed to amplify 5 kb of DNA from bacteriophage lambda using “PYROPHAGE”-brand Exo-DNA polymerase (SEQ ID NO: 18) (FIG. 8, lane 2), the Sac7d-V26/A29 protein (SEQ ID NO: 34) fused to the amino terminus of PYROPHAGE Exo-DNA polymerase (FIG. 8, lane 3), and TmaCsp (SEQ ID NO: 26) fused to the amino terminus of PYROPHAGE Exo-DNA polymerase (FIG. 8, lane 4). Fifty microliter reactions containing 1× “PYROPHAGE”-brand PCR Buffer (Lucigen), 5 units of the polymerase (both fusion and non-fusion), 10 ng lambda DNA (Promega, Madison, Wis.), 200 μM dNTPs (Takara Bio Inc., Tsu, Shiga, Japan), and 0.1 μM primers 5′-GAAGAGGTGGCGCGTAACGCGTCC-3′ (SEQ ID NO: 75) and 5′-GATGACATGCTTGTTTCATCAGGTG-3′ (SEQ ID NO: 76) were cycled at 94° C. for 2 mM and 30 cycles of 94° C. for 15 sec, 60° C. for 15 sec, and 72° C. for 5 mM. As shown in FIG. 8, both the Sac7d and the TmaCsp fusion proteins amplified DNA more effectively than the non-fusion polymerase. The Sac7d and the TmaCsp fusion proteins were equally effective in amplifying DNA.

This example shows that the fusion proteins comprising different nucleic acid-binding domains appended to a polymerase domain are equally effective in amplifying DNA in colony PCR and that both are more effective than the conventional polymerase.

### Example 5

To determine whether the fusion proteins described herein have a greater affinity than polymerases not fused to a nucleic acid binding domain, primer extension and gel shift assays were performed.

The following polymerases were incubated in a reaction mix containing bacteriophage M13 ssDNA (GenBank Acc. No. X02513) and 1× ThermoPol buffer (10 mM KCl, 20 mM Tris-HCl [pH 8.8], 10 mM (NH4) 2SO4, 2 mM MgSO4, 0.1% Triton X-100, 0.1 mg/ml BSA) with (FIG. 9, lanes 2-7) or without (FIG. 9, lanes 8-13) a primer (5′-CGC CAG GGT TTT CCC AGT CAC GAC-3′; SEQ ID NO: 77):
1. Bst DNA polymerase (FIG. 9, lanes 2 and 8);
2. No enzyme (FIG. 9, lanes 3 and 9);
3. Klenow exo-DNA polymerase (SEQ ID NO: 10) (FIG. 9, lanes 4 and 10);
4. Klenow exo-DNA polymerase fused to Tbr SSB protein at its carboxy terminus (SEQ ID NO: 60) (FIG. 9, lane 5 and 11);
5. T4 exo-DNA polymerase (SEQ ID NO: 8) (FIG. 9, lanes 6 and 12); or
6. T4 exo-DNA polymerase fused to Tbr SSB protein at its carboxy terminus (SEQ ID NO: 64) (FIG. 9, lanes 7 and 13).
   FIG. 9 shows that the lanes with polymerases fused to nucleic acid binding proteins (lanes 5 and 7) displayed a mobility shift compared to lanes with polymerases not fused to nucleic acid binding proteins (lanes 4 and 6). FIG. 9 also shows that the lanes with polymerases fused to nucleic acid binding proteins (lanes 11 and 13) displayed higher molecular weight nucleic acid species than lanes with polymerases not fused to nucleic acid binding proteins (lanes 10 and 12).

These data indicate that the polymerases fused to nucleic acid binding proteins have a greater affinity for DNA than polymerases not fused to nucleic acid binding proteins.

### REFERENCES

Baker T A and Bell S P (1998) “Polymerases and the replisome: machines within machines.” Cell 92: 295-305.
Bandzulis R J, Swanson M S, and Dreyfuss G (1989) “RNA-binding proteins as developmental regulators.” Genes & Development 3: 431-437.
Baumann H, Knapp S, Lundbäck T, Ladenstein R, and Härd T (1994) “Solution structure and DNA binding properties of a thermostable protein from the archaeon Sulfolobus solfataricus.” Nature Structural Biology 1: 808-819.
Borjac-Natour M J, Petrov V M, and Karam J M (2004) “Divergence of the mRNA targets for the Ssb proteins of bacteriophages T4 and RB69.” Virology Journal 1: 4doi:10.1186/1743-422X-1-4.
Chen T, Amons R, Clegg J S, Warner A H, and MacRae T H (2003) “Molecular characterization of artemin and ferritin from Artemia franciscana.” Eur. J. Biochem. 270: 137-145.
Chen C Y, Ko T P, Lin T W, Chou C C, and Wang A H J (2005) “Probing the DNA kink structure induced by the hyperthermophilic chromosomal protein Sac7d.” Nucleic Acids Res. 33: 430-438.
Chen Y and Varani G (2005) “Protein families and RNA recognition.” FEBS J. 272: 2088-2097.
Coté M L and Roth M J (2008) “Murine leukemia virus reverse transcriptase: structural comparison with HIV-1 reverse transcriptase.” Virus Res. 134: 186-202.
Davidson J F, Fox R, Harris D D, Lyons-Abbott S, and Loeb L A (2003) “Insertion of the T3 DNA polymerase thioredoxin binding domain enhances the processivity and fidelity of Taq DNA polymerase.” Nucleic Acids Res. 31: 4702-4709.
Dabrowski S and Kur J (1998) “Recombinant His-tagged DNA polymerase. I. Cloning, purification and partial characterization of Thermus thermophilus recombinant DNA polymerase.” Acta Biochimica Polonica 45: 653-660.
Delarue M, Poch O, Tordo N, Moras D, and Argos P (1990) “An attempt to unify the structure of polymerases.” Protein Engineering 3: 461-467.
Delbrück H, Mueller D, Perl D, Schmid F X, and Heinemann U (2001) “Crystal structures of mutant forms of Bacillus caldolyticus cold shock protein differing in thermal stability.” J. Mol. Biol. 313: 359-369.
Donald R G K and Jackson A O (1996) “RNA-binding activities of barley stripe mosaic virus γb fusion proteins.” J. Gen. Virology 77: 879-888.
Feng W, Tejero R, Zimmerman D E, Inouye M, and Montelione G T (1998) “Solution structure and backbone dynamics of the major cold-shock protein (CspA) from Escherichia coli: evidence for conformational dynamics in the single-stranded RNA-binding site.” Biochemistry 37: 10,881-10,896.
Gill P and Ghaemi A (2008) “Nucleic acid isothermal amplification technologies: a review.” Nucleosides, Nucleotides and Nucleic Acids 27: 224-243.
Graumann P, Wendrich T M, Weber M H, Schröder K, and Marahiel M A (1997) “A family of cold shock proteins in Bacillus subtilus is essential for cellular growth and for efficient protein synthesis at optimal and low temperatures.” Molecular Microbiology 25: 741-756.
Grote M, Dijk J, and Reinhardt R (1986) “Ribosomal and DNA binding proteins of the thermoacidophilic archaebacterium Sulfolobus acidocaldarius.” Biochim. Biophys. Acta 873: 405-413.
Guo R, Xue H, and Huang L (2003) “Ssh10b, a conserved thermophilic archaeal protein, binds RNA in vivo.” Molecular Microbiology 50: 1605-1615.
Guo L, Feng Y, Zhang Z, Yao H, Luo Y, Wang J, and Huang L (2008) “Biochemical and structural characterization of Cren7, a novel chromatin protein conserved among Crenarchaea.” Nucleic Acids Res. 36: 1129-1137.
Herschlag D, Khosla M, Tsuchihashi Z, and Karpel R L (1994) “An RNA chaperone activity of non-specific RNA binding proteins in hammerhead ribozyme catalysis.” EMBO J. 13: 2913-2924.
Jiang W, Hou Y, and Inouye M (1997) “CspA, the major cold-shock protein of Escherichia coli, is an RNA chaperone.” J. Biol. Chem. 272: 196-202.
Jung A, Bamann C, Kremer W, Kalbitzer R, and Brunner E (2004) “High-temperature solution NMR structure of TmCsp.” Protein Science 13: 342-350.
Kerr I D, Wadsworth R I M, Cubeddu L, Blankenfeldt W, Naismith J H, and White M F (2003) “Insights into ssDNA recognition by the OB fold from a structural and thermodynamic study of Sulfolobus SSB protein.” EMBO J. 22: 2561-2570.
Landsman D (1992) “RNP-1, an RNA-binding motif is conserved in the DNA-binding cold shock domain.” Nucleic Acids Res. 20: 2861-2864.
Le Grice S F and Grüninger-Leitch F (1990) “Rapid purification of homodimer and heterodimer HIV-1 reverse transcriptase by metal chelate affinity chromatography.” Eur. J. Biochem. 187: 307-314.
Melekhovets Y F and Joshi S (1996) “Fusion with an RNA binding domain to confer target RNA specificity to an RNase: design and engineering of Tat-RNase H that specifically recognizes and cleaves HIV-1 RNA in vitro.” Nucleic Acids Res. 24: 1908-1912.
Mizuno Y, Carninci P, Okazaki Y, Tateno M, Kawai J, Amanuma H, Muramatsu M, and Hayashizaki Y (1999) “Increased specificity of reverse transcription priming by trehalose and oligo-blockers allows high-efficiency window separation of mRNA display.” Nucleic Acids Res. 27: 1345-1349.
Mötz M, Kober I, Girardot C, Loeser E, Bauer U, Albers M, Moeckel G, Minch E, Voss H, Kilger C, and Koegl M (2002) “Elucidation of an archaeal replication protein network to generate enhanced PCR enzymes.” J. Biol. Chem. 277: 16179-16188.
Mueller U, Perl D, Schmid F X, and Heinemann U (2000) “Thermal stability and atomic resolution crystal structure of the Bacillus caldolyticus cold shock protein.” J. Mol. Biol. 297: 975-988.
Murzin A G (1993) “OB (Oligonucleotide/oligosaccharide binding)-fold: common structural and functional solution for non-homologous sequences.” EMBO J. 12: 861-867.
Myers T W and Gelfand D H (1991) “Reverse transcription and amplification by a Thermus thermophilus DNA polymerase.” Biochemistry 30: 7661-7666.
Newkirk K, Feng W, Jiang W, Tejero R, Emerson S D, Inouye M, and Montelione G T (1994) “Solution NMR structure of the major cold shock protein (CspA) from Escherichia coli: Identification of a binding epitope for DNA.” Proc. Nat. Acad. Sciences USA 91: 5114-5118.
Notomi T, Okayama H, Masubuchi H, Yonekawa T, Watanabe K, Amino N, and Hase T (2000) “Loop-mediated isothermal amplification of DNA.” Nucleic Acids Res. 28: e63.
Phadtare S and Inouye M (1999) “Sequence-selective interactions with RNA by CspB, CspC, and CspE, members of the CspA family of Escherichia coli.” Molecular Microbiology 33: 1004-1014.
Phadtare S, Hwang J, Sevferinov K, and Inouye M (2003) “CspB and CspL, thermo-stable cold-shock proteins from Thermotoga maritima.” Genes to Cells 8: 801-810.
Ross I M, Wadsworth M, and White M F (2001) “Identification and properties of the crenarchal single-stranded DNA binding protein from Sulfolobus solfataricus.” Nucleic Acids Res. 29: 4914-4920.
Saiki, R, Scharf, S, Faloona, F, Mullis, K, Horn, G, and Erlich, H (1985). Enzymatic amplification of beta-globin genomic sequences and restriction site analysis for diagnosis of sickle cell anemia.” Science 230: 1350-1354.
Schindelin H, Jiang W, Inouye M, and Heinemann U (1994) “Crystal structure of CspA, the major cold shock protein of Escherichia coli.” Proc. Nat. Acad. Sciences USA 91: 5119-5123.
Shehi E, Serina S, Fumagalli G, Vanoni M, Consonni R, Zetta L, Deho G, Tortora P, and Fusi P (2001) “The Sso7d DNA-binding protein from Sulfolobus solfataricus has ribonuclease activity.” FEBS Letters 497: 131-136.
Smith B J and Bailey J M (1979) “The binding of an avian myeloblastosis virus basic 12,000 dalton protein to nucleic acids.” Nucleic Acids Res. 7: 2055-2072.
Stammers D K, Tisdale M, Court S, Parmar V, Bradley C, and Ross C K (1991) “Rapid purification and characterization of HIV-1 reverse transcriptase and RNAseH engineered to incorporate a C-terminal tripeptide alpha-tubulin epitope.” FEBS Letters 283: 298-302.
Steitz T A (1999) “DNA Polymerases: Structural Diversity and Common Mechanisms.” J. Biol. Chem. 274: 17395-17398.
Steitz T A (2006) “Visualizing polynucleotide polymerase machines at work.” EMBO J. 25: 3458-3468.
Sun S, Geng L, and Shamoo Y (2006) “Structure and enzymatic properties of a chimeric bacteriophage RB69 polymerase and single-stranded DNA binding protein with increased processivity.” Proteins 65: 231-238.
Sykora K W and Moelling K (1981) “Properties of the avian viral protein p12.” J. Gen. Virology 55: 379-391.
Tanese N, Roth M, and Goff S P (1985) “Expression of enzymatically active reverse transcriptase in Escherichia coli.” Proc. Nat. Acad. Sciences USA 82: 4944-4945.
Theobald D L, Mitton-Fry R M, and Wiittke D S (2003) “Nucleic Acid Recognition by OB-Fold Proteins.” Ann. Rev. Biophys. Biomolecular Structure 32: 115-133.
Wang A, Prosen D, Mei L, Sullivan J C, Finney M, and Vander Horn P B (2004) “A novel strategy to engineer DNA polymerases for enhanced processivity and improved performance in vitro.” Nucleic Acids Res. 32: 1197-1207.
Wang N, Yamanaka K, and Inouye M (2000) “Acquisition of double-stranded DNA-binding ability in a hybrid protein between Escherichia coli CspA and the cold shock domain of human YB-1.” Molecular Microbiology 38: 526-534.
Weber M H W and Marahiel M (2002) “Coping with the cold: the cold shock response in the Gram-positive soil bacterium Bacillus subtilus.” Phil. Trans. Royal Soc. London B 357: 895-907.
Yasukawa K, Nemoto D, and Inouye K (2008) “Comparison of the thermal stabilities of reverse transcriptases from avian myeloblastosis virus and Moloney murine leukaemia virus.” J. Biochemistry 143: 261-268.

What is claimed is:

1. A fusion protein comprising a first polypeptide domain operationally connected to or directly linked to a second polypeptide domain;
wherein the first polypeptide domain comprises an oligonucleotide/oligosaccharide binding (OB) fold and at least one RNA binding motif; and
wherein the second polypeptide domain comprises a polymerase domain.

2. The fusion protein of claim 1 wherein the at least one RNA binding motif is selected from the group consisting of GYGFI, VFVHW, and VFVHF.

3. The fusion protein of claim 1 wherein the at least one RNA binding motif is contained on beta sheet β2 or beta sheet β3 of the OB fold.

4. The fusion protein of claim 1 wherein the first polypeptide domain comprises at least two RNA binding motifs.

5. The fusion protein of claim 4 wherein a first of the at least two RNA binding motifs is contained on beta sheet β2 of the OB fold and a second of the at least two RNA binding motifs is contained on beta sheet β3 of the OB fold.

6. The fusion protein of claim 1 wherein the first polypeptide domain further comprises a DNA binding motif.

7. The fusion protein of claim 6 wherein the DNA binding motif is between beta sheets β3 and β4 of the OB fold.

8. The fusion protein of claim 6 wherein the DNA binding motif is selected from the group consisting of AIEM, AIQG, AIQN, VGKM, VGKA, AGKA, and LAPKGRKGVKI.

9. The fusion protein of claim 1 wherein the first polypeptide domain is thermostable.

10. The fusion protein of claim 1 wherein the first polypeptide domain is at least 60% identical to a sequence selected from the group consisting of SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, and SEQ ID NO: 70.

11. The fusion protein of claim 1 wherein the first polypeptide domain is at least 95% identical to SEQ ID NO: 70.

12. The fusion protein of claim 1 wherein the polymerase domain is a DNA-dependent DNA polymerase.

13. The fusion protein of claim 1 wherein the polymerase domain is an RNA-dependent DNA polymerase.

14. The fusion protein of claim 1 wherein the polymerase domain is a Klenow fragment of a DNA polymerase.

15. The fusion protein of claim 1 wherein the polymerase domain is thermostable.

16. The fusion protein of claim 1 wherein the second polypeptide domain is at least 60% identical to a sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, and SEQ ID NO: 24.

17. The fusion protein of claim 1 further comprising a linker between the first polypeptide domain and the second polypeptide domain.

18. The fusion protein of claim 1 further comprising a third polypeptide domain operationally connected to the first polypeptide domain and the second polypeptide domain or directly linked to the first polypeptide domain or the second polypeptide domain, wherein the third polypeptide domain comprises a motif selected from the group consisting of at least one RNA binding motif and at least one DNA binding motif.

19. The fusion protein of claim 18 wherein the third polypeptide domain comprises an OB fold.

20. The fusion protein of claim 19 wherein the third polypeptide domain is at least 80% identical to a sequence selected from the group consisting of SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, and SEQ ID NO: 70.

21. A nucleic acid that encodes a fusion protein as recited in claim 1.

22. A method of synthesizing a nucleic acid comprising contacting a nucleic acid template with a fusion protein as recited in claim 1.

23. The method of claim 22 wherein the contacting is performed in a procedure selected from the group consisting of measuring levels of mRNA in a cell extract, sequencing a nucleic acid, synthesizing DNA polymers, reverse transcribing RNA polymers to produce complementary DNA (cDNA), amplifying DNA in a polymerase chain reaction (PCR), amplifying DNA in an isothermal nucleotide amplification reaction, and reverse transcribing RNA and amplifying DNA in a one-tube, one-enzyme reverse transcription-polymerase chain reaction (RT-PCR).


SEQUENCE LISTING (81 sequences)

SEQ ID NO: 1 - DNA, 2505 bp, Thermus thermophilus
atggaggcgatgcttccgctctttgaacccaaaggccgggtcctcctggcggacggccaccacctggcctaccgcaccttcttcgccctgaagggcctcaccacgagccggggcgaaccggtgcaggcggtctacggcttcgccaagagcctcctcaaggccctgaaggaggacgggtacaaggccgtcttcgtggtctttgacgccaaggccctctccttccgccacgaggcctacgaggcctacaaggcggggagggccccgacccccgaggacttcccccggcagctcgccctcatcaaggagctggtggacctcctggggtttacccgcctcgaggtccccggctacgaggcggacgacgtcctcgccaccctggccaagaaggcggaaaaagaagggtacgaggtgcgcatcctcaccgccgaccgggacctctaccagctcgtctccgactgcgtcgccgtcctccaccccgagggccacctcatcaccccggagtggctttgggagaagtacggcctcaggccggagcagtgggtggacttccgcgccctcgtgggggacccctccgacaacctccccggggtcaagggcatcggggagaagaccgccctcaagctcctcaaggagtggggaagcctggaaaacctcctcaagaacctggaccgggtgaagccggaaaacgtccgggagaagatcaaggcccacctggaagacctcaggctctccttggggctctcccgggtgcgcaccgacctccccctggaggtggacctcgcccaggggcgggagcccgaccgggaggggcttagggccttcctggagaggctggagttcggcagcctcctccacgagttcggcctcctggaggcccccgcccccctggaggaggccccctggcccccgccggaaggggccttcgtgggcttcgtcctctcccgccccgagcccatgtgggcggagcttaaagccctggccgcctgcagggacggccgggtgcaccgggcagcggaccccttggcggggctaaaggacctcaaggaggtccggggcctcctcgccaaggacctcgccgtcttggcctcgagggaggggctagacctcgtgcccggggacgaccccatgctcctcgcctacctcctggacccctccaacaccacccccgagggggtggcgcggcgctacggaggggagtggacggaggacgccgcccaccgggccctcctctcggagaggctccatcagaacctccttaagcgcctccagggggaggagaagctcctttggctctaccacgaggtggaaaagcccctctcccgggtcctggcccacatggaggccaccggggtacggctggacgtggcctaccttcaggccctttccctggagcttgcggaggagatccgccgcctcgaggaggaggtcttccgcttggcgggccaccccttcaacctcaactcccgagaccagctggaaagggtgctctttgacgagcttaggcttcccgccttggggaagacgcaaaagacgggcaagcgctccaccagcgccgcggtgctggaggccctacgggaggcccaccccatcgtggagaagatcctccagcaccgggagctcaccaagctcaagaacacctacgtggaccccctcccaagcctcgtccacccgaggacgggccgcctccacacccgcttcaaccagacggccacggccacagggaggcttagtagctccgaccccaacctgcagaacatccccgtccgcacccccttgggccagaggatccgccgggccttcgtggccgaggcgggatgggcgttggtggccctggactatagccagatagagctccgcgtcctcgcccacctctccggggacgagaacctgatcagggtcttccaggaggggaaggacattcacacccagaccgcaagctggatgttcggcgtccccccggaggccgtggaccccctgatgcgccgggcggccaagacggtgaacttcggcgtcctctacggcatgtccgcccaccggctctcccaggagctctccatcccctacgaggaggcctcggccttcattgagcgctacttccagagcttccccaaggtgcgggcctggatagaaaagaccctggaggaggggaggaagcggggctacgtggaaaccctcttcggaagaaggcgctacgtgcccgacctcaacgcccgggtgaagagcgtcagggaggccgcggagcgcatggccttcaacatgcccgtccagggcaccgccgccgacctcatgaagctcgccatggtgaagctcttcccccgcctccggcagatgggggcccgcatgctcctccaggtccacgacgagctcctcctggaggccccccaagcgcgggccgaggaggtggcggctttggccaaggaggccatggagaaggcctatcccctcgccgtgcccctggaggtggaggcggggatcggggaggactggctttccgccaagggttag

SEQ ID NO: 2 - protein, 834 aa, Thermus thermophilus
MEAMLPLFEPKGRVLLADGHHLAYRTFFALKGLTTSRGEPVQAVYGFAKSLLKALKEDGYKAVFVVFDAKALSFRHEAYEAYKAGRAPTPEDFPRQLALIKELVDLLGFTRLEVPGYEADDVLATLAKKAEKEGYEVRILTADRDLYQLVSDCVAVLHPEGHLITPEWLWEKYGLRPEQWVDFRALVGDPSDNLPGVKGIGEKTALKLLKEWGSLENLLKNLDRVKPENVREKIKAHLEDLRLSLGLSRVRTDLPLEVDLAQGREPDREGLRAFLERLEFGSLLHEFGLLEAPAPLEEAPWPPPEGAFVGFVLSRPEPMWAELKALAACRDGRVHRAADPLAGLKDLKEVRGLLAKDLAVLASREGLDLVPGDDPMLLAYLLDPSNTTPEGVARRYGGEWTEDAAHRALLSERLHQNLLKRLQGEEKLLWLYHEVEKPLSRVLAHMEATGVRLDVAYLQALSLELAEEIRRLEEEVFRLAGHPFNLNSRDQLERVLFDELRLPALGKTQKTGKRSTSAAVLEALREAHPIVEKILQHRELTKLKNTYVDPLPSLVHPRTGRLHTRFNQTATATGRLSSSDPNLQNIPVRTPLGQRIRRAFVAEAGWALVALDYSQIELRVLAHLSGDENLIRVFQEGKDIHTQTASWMFGVPPEAVDPLMRRAAKTVNFGVLYGMSAHRLSQELSIPYEEASAFIERYFQSFPKVRAWIEKTLEEGRKRGYVETLFGRRRYVPDLNARVKSVREAAERMAFNMPVQGTAADLMKLAMVKLFPRLRQMGARMLLQVHDELLLEAPQARAEEVAALAKEAMEKAYPLAVPLEVEAGIGEDWLSAKG

SEQ ID NO: 3 - DNA, 2514 bp, Thermus aquaticus
atgaccatgattacgaattcggggatgctgcccctctttgagcccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgccctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgttcggcgtcccccgggaggccgtggaccccctgatgcgccgggcggccaagaccatcaactacggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtga

SEQ ID NO: 4 - protein, 837 aa, Thermus aquaticus
MTMITNSGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHALHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 5 - DNA, 1635 bp, Thermus aquaticus
atgagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgttcggcgtcccccgggaggccgtggaccccctgatgcgccgggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtga

SEQ ID NO: 6 - protein, 544 aa, Thermus aquaticus
MSPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 7 - DNA, 2718 bp, Bacteriophage T4
atggggcatcaccatcaccatcacaaagaattttatatctctattgaaacagtcggaaataacattgttgaacgttatattgatgaaaatggaaaggaacgtacccgtgaagtagaatatcttccaactatgtttaggcattgtaaggaagagtcaaaatacaaagacatctatggtaaaaactgcgctcctcaaaaatttccatcaatgaaagatgctcgagattggatgaagcgaatggaagacatcggtctcgaagctctcggtatgaacgattttaaactcgcttatataagtgatacatatggttcagaaattgtttatgaccgaaaatttgttcgtgtagctaactgtgacattgaggttactggtgataaatttcctgacccaatgaaagcagaatatgaaattgatgctatcactcattacgattcaattgacgatcgtttttatgttttcgaccttttgaattcaatgtacggttcagtatcaaaatgggatgcaaagttagctgctaagcttgactgtgaaggtggtgatgaagttcctcaagaaattcttgaccgagtaatttatatgccattcgataatgagcgtgatatgctcatggaatatatcaatctttgggaacagaaacgacctgctatttttactggttggaatattgaggggtttgccgttccgtatatcatgaatcgtgttaaaatgattctgggtgaacgtagtatgaaacgtttctctccaatcggtcgggtaaaatctaaactaattcaaaatatgtacggtagcaaagaaatttattctattgatggcgtatctattcttgattatttagatttgtacaagaaattcgcttttactaatttgccgtcattctctttggaatcagttgctcaacatgaaaccaaaaaaggtaaattaccatacgacggtcctattaataaacttcgtgagactaatcatcaacgatacattagttataacatcattgacgtagaatcagttcaagcaatcgataaaattcgtgggtttatcgatctagttttaagtatgtcttattacgctaaaatgcctttttctggtgtaatgagtcctattaaaacttgggatgctattatttttaactcattgaaaggtgaacataaggttattcctcaacaaggttcgcacgttaaacagagttttccgggtgcatttgtgtttgaacctaaaccaattgcacgtcgatacattatgagttttgacttgacgtctctgtatccgagcattattcgccaggttaacattagtcctgaaactattcgtggtcagtttaaagttcatccaattcatgaatatatcgcaggaacagctcctaaaccgagtgatgaatattcttgttctccgaatggatggatgtatgataaacatcaagaaggtatcattccaaaggaaatcgctaaagtatttttccagcgtaaagactggaaaaagaaaatgttcgctgaagaaatgaatgccgaagctattaaaaagattattatgaaaggcgcagggtcttgttcaactaaaccagaagttgaacgatatgttaagttcagtgatgatttcttaaatgaactatcgaattacaccgaatctgttctcaatagtctgattgaagaatgtgaaaaagcagctacacttgctaatacaaatcagctgaaccgtaaaattctcattaacagtctttatggtgctcttggtaatattcatttccgttactatgatttgcgaaatgctactgctatcacaattttcggccaagtcggtattcagtggattgctcgtaaaattaatgaatatctgaataaagtatgcggaactaatgatgaagatttcattgcagcaggtgatactgattcggtatatgtttgcgtagataaagttattgaaaaagttggtcttgaccgattcaaagagcagaacgatttggttgaattcatgaatcagttcggtaagaaaaagatggaacctatgattgatgttgcatatcgtgagttatgtgattatatgaataaccgcgagcatctgatgcatatggaccgtgaagctatttcttgccctccgcttggttcaaagggcgttggtggattttggaaagcgaaaaagcgttatgctctgaacgtttatgatatggaagataagcgatttgctgaaccgcatctaaaaatcatgggtatggaaactcagcagagttcaacaccaaaagcagtgcaagaagctctcgaagaaagtattcgtcgtattcttcaggaaggtgaagagtctgtccaagaatactacaagaacttcgagaaagaatatcgtcaacttgactataaagttattgctgaagtaaaaactgcgaacgatatagcgaaatatgatgataaaggttggccaggatttaaatgcccgttccatattcgtggtgtgctaacttatcgtcgagctgttagcggtttaggtgtagctccaattttggatggaaataaagtaatggttcttccattacgtgaaggaaatccatttggtgacaagtgcattgcttggccatcgggtacagaacttccaaaagaaattcgttctgatgtgctatcttggattgaccactcaactttgttccaaaaatcgtttgttaaaccgcttgcgggtatgtgtgaatcggctggcatggactatgaagaaaaagcttcgttagacttcctgtttggctga

SEQ ID NO: 8 - protein, 898 aa, Bacteriophage T4
MKEFYISIETVGNNIVERYIDENGKERTREVEYLPTMFRHCKEESKYKDIYGKNCAPQKFPSMKDARDWMKRMEDIGLEALGMNDFKLAYISDTYGSEIVYDRKFVRVANCDIEVTGDKFPDPMKAEYEIDAITHYDSIDDRFYVFDLLNSMYGSVSKWDAKLAAKLDCEGGDEVPQEILDRVIYMPFDNERDMLMEYINLWEQKRPAIFTGWNIEGFAVPYIMNRVKMILGERSMKRFSPIGRVKSKLIQNMYGSKEIYSIDGVSILDYLDLYKKFAFTNLPSFSLESVAQHETKKGKLPYDGPINKLRETNHQRYISYNIIDVESVQAIDKIRGFIDLVLSMSYYAKMPFSGVMSPIKTWDAIIFNSLKGEHKVIPQQGSHVKQSFPGAFVFEPKPIARRYIMSFDLTSLYPSIIRQVNISPETIRGQFKVHPIHEYIAGTAPKPSDEYSCSPNGWMYDKHQEGIIPKEIAKVFFQRKDWKKKMFAEEMNAEAIKKIIMKGAGSCSTKPEVERYVKFSDDFLNELSNYTESVLNSLIEECEKAATLANTNQLNRKILINSLYGALGNIHFRYYDLRNATAITIFGQVGIQWIARKINEYLNKVCGTNDEDFIAAGDTDSVYVCVDKVIEKVGLDRFKEQNDLVEFMNQFGKKKMEPMIDVAYRELCDYMNNREHLMHMDREAISCPPLGSKGVGGFWKAKKRYALNVYDMEDKRFAEPHLKIMGMETQQSSTPKAVQEALEESIRRILQEGEESVQEYYKNFEKEYRQLDYKVIAEVKTANDIAKYDDKGWPGFKCPFHIRGVLTYRRAVSGLGVAPILDGNKVMVLPLREGNPFGDKCIAWPSGTELPKEIRSDVLSWIDHSTLFQKSFVKPLAGMCESAGMDYEEKASLDFLFG

SEQ ID NO: 9 - DNA, 1821 bp, Escherichia coli
atggtgatttcttatgacaactacgtcaccatccttgatgaagaaacactgaaagcgtggattgcgaagctggaaaaagcgccggtatttgcatttgctaccgcaaccgacagccttgataacatctctgctaacctggtcgggctttcttttgctatcgagccaggcgtagcggcatatattccggttgctcatgattatcttgatgcgcccgatcaaatctctcgcgagcgtgcactcgagttgctaaaaccgctgctggaagatgaaaaggcgctgaaggtcgggcaaaacctgaaatacgatcgcggtattctggcgaactacggcattgaactgcgtgggattgcgtttgataccatgctggagtcctacattctcaatagcgttgccgggcgtcacgatatggacagcctcgcggaacgttggttgaagcacaaaaccatcacttttgaagagattgctggtaaaggcaaaaatcaactgacctttaaccagattgccctcgaagaagccggacgttacgccgccgaagatgcagatgtcaccttgcagttgcatctgaaaatgtggccggatctgcaaaaacacaaagggccgttgaacgtcttcgagaatatcgaaatgccgctggtgccggtgctttcacgcattgaacgtaacggtgtgaagatcgatccgaaagtgctgcacaatcattctgaagagctcacccttcgtctggctgagctggaaaagaaagcgcatgaaattgcaggtgaggaatttaacctttcttccaccaagcagttacaaaccattctctttgaaaaacagggcattaaaccgctgaagaaaacgccgggtggcgcgccgtcaacgtcggaagaggtactggaagaactggcgctggactatccgttgccaaaagtgattctggagtatcgtggtctggcgaagctgaaatcgacctacaccgacaagctgccgctgatgatcaacccgaaaaccgggcgtgtgcatacctcttatcaccaggcagtaactgcaacgggacgtttatcgtcaaccgatcctaacctgcaaaacattccggtgcgtaacgaagaaggtcgtcgtatccgccaggcgtttattgcgccagaggattatgtgattgtctcagcggactactcgcagattgaactgcgcattatggcgcatctttcgcgtgacaaaggcttgctgaccgcattcgcggaaggaaaagatatccaccgggcaacggcggcagaagtgtttggtttgccactggaaaccgtcaccagcgagcaacgccgtagcgcgaaagcgatcaactttggtctgatttatggcatgagtgctttcggtctggcgcggcaattgaacattccacgtaaagaagcgcagaagtacatggacctttacttcgaacgctaccctggcgtgctggagtatatggaacgcacccgtgctcaggcgaaagagcagggctacgttgaaacgctggacggacgccgtctgtatctgccggatatcaaatccagcaatggtgctcgtcgtgcagcggctgaacgtgcagccattaacgcgccaatgcagggaaccgccgccgacattatcaaacgggcgatgattgccgttgatgcgtggttacaggctgagcaaccgcgtgtacgtatgatcatgcaggtacacgatgaactggtatttgaagttcataaagatgatgttgatgccgtcgcgaagcagattcatcaactgatggaaaactgtacccgtctggatgtgccgttgctggtggaagtggggagtggcgaaaactgggatcaggcgcactaa

SEQ ID NO: 10 - protein, 606 aa, Escherichia coli
MVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAH

SEQ ID NO: 11 - DNA, 2682 bp, Avian Myeloblastosis Virus
atagggagggccactgttcttactgttgcgctacatctggctattccgctcaaatggaagccaaaccacacgcctgtgtggattgaccagtggccccttcctgaaggtaaacttgtagcgctaacgcaattagtggaaaaagaattacagttaggacatatagaaccttcacttagttgctggaacacacctgtctttgtgatccggaaggcttccgggtcttaccgcttattgcatgacttgcgcgctgttaacgctaagcttgttccttttggggccgtccaacagggggcgccagttctctccgcgctcccgcgtggttggcccctgatggttctagacctcaaggattgcttcttttctattcctcttgcggaacaagatcgcgaagcttttgcatttacgctcccctccgtgaataaccaggcccccgctcgaaggttccaatggaaggtcttgccccaagggatgacctgttctcccactatctgtcagttgatagtgggtcaaatacttgagcccttgcgactcaagcacccatctctgcgcatgttgcattatatggatgatcttttgctagccgcctcaagtcatgatgggttggaagcggcaggggaggaggttatcagtacattggaaagagccgggttcaccatttcgcctgataaggtccagagggagcccggagtacaatatcttgggtacaagttaggtagtacgtatgtagcacccgtaggcctggtagcagaacccaggatagccaccttgtgggatgttcagaagctggtggggtcacttcagtggcttcgcccagcgttaggaatcccgccacgactgaggggccccttttatgagcagttacgagggtcagatcctaacgaggcgagggaatggaatctagacatgaaaatggcctggagagagatcgtacggctcagcaccactgctgccttggaacgatgggaccctgccctgcctctggaaggagcggtcgctagatgtgaacagggggcaataggggtcctgggacagggactgtccacacacccaaggccatgtttgtggttattctccacccaacccaccaaggcgtttactgcttggttagaagtgctcacccttttgattactaagttacgtgcttcggcagtgcgaacctttggcaaggaggttgatatcctcctgttgcctgcatgctttcgggacgaccttccgctcccagaggggatcctgttagcccttagggggtttgcaggaaaaatcaggagtagtgacacgccatctatttttgacattgcgcgtccactgcatgtttctctgaaagtgagggttaccgaccaccctgtgccgggacccactgtctttactgacgcctcctcaagcacccataagggggtggtagtctggagggagggcccaaggtgggagataaaagaaatagctgatttgggggcaagtgtacaacaactggaagcacgggctgtggccatggcacttctgctgtggccgacaacgcccactaatgtagtgactgactctgcgtttgttgcgaaaatgttactcaagatgggacaggagggagtcccgtctacagcggcggctttcattttagaggatgcgttaagccaaaggtcagccatggccgccgttctccacgtgcggagtcattctgaagtgccagggtttttcacagaaggaaatgacgtggcagatagccaagccacctttcaagcgtatcccttgagagaggctaaagatctccataccgctctccatattggaccccgcgcgctatccaaagcgtgtaatatatctatgcagcaggctagggaggttgttcagacctgcccgcattgtaattcagcccctgcgttggaggccggggtaaaccctaggggtttgggacccttacagatatggcagacagactttacgcttgagcctagaatggccccccgttcctggctcgctgttactgtggataccgcctcatcggcgatagtcgtaactcagcatggccgtgtcacatcggttgctgcacaacatcattgggccacggctatcgccgttttgggaagaccaaaggccataaaaacagataacgggtcctgcttcacgtctaaatccacgcgagagtggctcgcgagatgggggatagcacacaccaccgggattccgggtaattcccagggtcaagctatggtagagcgggccaaccggctcctgaaagataagatccgtgtgcttgcggagggggatggctttatgaaaagaatccccaccagcaaacagggggaactattagccaaggcaatgtatgccctcaatcactttgagcgtggtgaaaacacaaaaacaccgatacaaaaacactggagacctaccgttcttacagaaggacccccggttaagatacgaatagagacaggggagtgggaaaaaggatggaacgtgctggtctggggacgaggttatgccgctgtgaaaaacagggacactgataaggttatttgggtaccctctcgaaaagttaaaccggacatcgcccaaaaggatgaggtgactaagaaagatgaggcgagccctctttttgcaggctggaggcacatagataagagaattatcactctacattcatctttctcaaagattaatctacttgtgtgttttatatttcattag

SEQ ID NO: 12 - protein, 893 aa, Avian Myeloblastosis Virus
IGRATVLTVALHLAIPLKWKPNHTPVWIDQWPLPEGKLVALTQLVEKELQLGHIEPSLSCWNTPVFVIRKASGSYRLLHDLRAVNAKLVPFGAVQQGAPVLSALPRGWPLMVLDLKDCFFSIPLAEQDREAFAFTLPSVNNQAPARRFQWKVLPQGMTCSPTICQLIVGQILEPLRLKHPSLRMLHYMDDLLLAASSHDGLEAAGEEVISTLERAGFTISPDKVQREPGVQYLGYKLGSTYVAPVGLVAEPRIATLWDVQKLVGSLQWLRPALGIPPRLRGPFYEQLRGSDPNEAREWNLDMKMAWREIVRLSTTAALERWDPALPLEGAVARCEQGAIGVLGQGLSTHPRPCLWLFSTQPTKAFTAWLEVLTLLITKLRASAVRTFGKEVDILLLPACFRDDLPLPEGILLALRGFAGKIRSSDTPSIFDIARPLHVSLKVRVTDHPVPGPTVFTDASSSTHKGVVVWREGPRWEIKEIADLGASVQQLEARAVAMALLLWPTTPTNVVTDSAFVAKMLLKMGQEGVPSTAAAFILEDALSQRSAMAAVLHVRSHSEVPGFFTEGNDVADSQATFQAYPLREAKDLHTALHIGPRALSKACNISMQQAREVVQTCPHCNSAPALEAGVNPRGLGPLQIWQTDFTLEPRMAPRSWLAVTVDTASSAIVVTQHGRVTSVAAQHHWATAIAVLGRPKAIKTDNGSCFTSKSTREWLARWGIAHTTGIPGNSQGQAMVERANRLLKDKIRVLAEGDGFMKRIPTSKQGELLAKAMYALNHFERGENTKTPIQKHWRPTVLTEGPPVKIRIETGEWEKGWNVLVWGRGYAAVKNRDTDKVIWVPSRKVKPDIAQKDEVTKKDEASPLFAGWRHIDKRIITLHSSFSKINLLVCFIFH

SEQ ID NO: 13 - DNA, 5214 bp, Moloney Murine Leukemia Virus
atgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccaaacctaaacctcaagttctttctgacagtggggggccgctcatcgacctacttacagaagaccccccgccttatagggacccaagaccacccccttccgacagggacggaaatggtggagaagcgacccctgcgggagaggcaccggacccctccccaatggcatctcgcctacgtgggagacgggagccccctgtggccgactccactacctcgcaggcattccccctccgcgcaggaggaaacggacagcttcaatactggccgttctcctcttctgacctttacaactggaaaaataataacccttctttttctgaagatccaggtaaactgacagctctgatcgagtctgttctcatcacccatcagcccacctgggacgactgtcagcagctgttggggactctgctgaccggagaagaaaaacaacgggtgctcttagaggctagaaaggcggtgcggggcgatgatgggcgccccactcaactgcccaatgaagtcgatgccgcttttcccctcgagcgcccagactgggattacaccacccaggcaggtaggaaccacctagtccactatcgccagttgctcctagcgggtctccaaaacgcgggcagaagccccaccaatttggccaaggtaaaaggaataacacaagggcccaatgagtctccctcggccttcctagagagacttaaggaagcctatcgcaggtacactccttatgaccctgaggacccagggcaagaaactaatgtgtctatgtctttcatttggcagtctgccccagacattgggagaaagttagagaggttagaagatttaaaaaacaagacgcttggagatttggttagagaggcagaaaagatctttaataaacgagaaaccccggaagaaagagaggaacgtatcaggagagaaacagaggaaaaagaagaacgccgtaggacagaggatgagcagaaagagaaagaaagagatcgtaggagacatagagagatgagcaagctattggccactgtcgttagtggacagaaacaggatagacagggaggagaacgaaggaggtcccaactcgatcgcgaccagtgtgcctactgcaaagaaaaggggcactgggctaaagattgtcccaagaaaccacgaggacctcggggaccaagaccccagacctccctcctgaccctagatgacggaggtcagggtcaggagcccccccctgaacccaggataaccctcaaagtcggggggcaacccgtcaccttcctggtagatactggggcccaacactccgtgctgacccaaaatcctggacccctaagtgataagtctgcctgggtccaaggggctactggaggaaagcggtatcgctggaccacggatcgcaaagtacatctagctaccggtaaggtcacccactctttcctccatgtaccagactgtccctatcctctgttaggaagagatttgctgactaaactaaaagcccaaatccactttgagggatcaggagctcaggttatgggaccaatggggcagcccctgcaagtgttgaccctaaatatagaagatgagcatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttgatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggacggcaggcttctgtcgcctctggatccctgggtttgcagaaatggcagcccccttgtaccctctcaccaaaacggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggttgctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctacacctcagaacattttcattacacagtgactgatataaaggacctaaccaagttgggggccatttatgataaaacaaagaagtattgggtctaccaaggaaaacctgtgatgcctgaccagtttacttttgaattattagactttcttcatcagctgactcacctcagcttctcaaaaatgaaggctctcctagagagaagccacagtccctactacatgctgaaccgggatcgaacactcaaaaatatcactgagacctgcaaagcttgtgcacaagtcaacgccagcaagtctgccgttaaacagggaactagggtccgcgggcatcggcccggcactcattgggagatcgatttcaccgagataaagcccggattgtatggctataaatatcttctagtttttatagataccttttctggctggatagaagccttcccaaccaagaaagaaaccgccaaggtcgtaaccaagaagctactagaggagatcttccccaggttcggcatgcctcaggtattgggaactgacaatgggcctgccttcgtctccaaggtgagtcagacagtggccgatctgttggggattgattggaaattacattgtgcatacagaccccaaagctcaggccaggtagaaagaatgaatagaaccatcaaggagactttaactaaattaacgcttgcaactggctctagagactgggtgctcctactccccttagccctgtaccgagcccgcaacacgccgggcccccatggcctcaccccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgacatggcgcgttcaacgctctcaaaaccccttaaaaataaggttaacccgcgaggccccctaa

SEQ ID NO: 14 - protein, 1737 aa, Moloney Murine Leukemia Virus
MGQTVTTPLSLTLGHWKDVERIAHNQSVDVKKRRWVTFCSAEWPTFNVGWPRDGTFNRDLITQVKIKVFSPGPHGHPDQVPYIVTWEALAFDPPPWVKPFVHPKPPPPLPPSAPSLPLEPPRSTPPRSSLYPALTPSLGAKPKPQVLSDSGGPLIDLLTEDPPPYRDPRPPPSDRDGNGGEATPAGEAPDPSPMASRLRGRREPPVADSTTSQAFPLRAGGNGQLQYWPFSSSDLYNWKNNNPSFSEDPGKLTALIESVLITHQPTWDDCQQLLGTLLTGEEKQRVLLEARKAVRGDDGRPTQLPNEVDAAFPLERPDWDYTTQAGRNHLVHYRQLLLAGLQNAGRSPTNLAKVKGITQGPNESPSAFLERLKEAYRRYTPYDPEDPGQETNVSMSFIWQSAPDIGRKLERLEDLKNKTLGDLVREAEKIFNKRETPEEREERIRRETEEKEERRRTEDEQKEKERDRRRHREMSKLLATVVSGQKQDRQGGERRRSQLDRDQCAYCKEKGHWAKDCPKKPRGPRGPRPQTSLLTLDDGGQGQEPPPEPRITLKVGGQPVTFLVDTGAQHSVLTQNPGPLSDKSAWVQGATGGKRYRWTTDRKVHLATGKVTHSFLHVPDCPYPLLGRDLLTKLKAQIHFEGSGAQVMGPMGQPLQVLTLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSPYTSEHFHYTVTDIKDLTKLGAIYDKTKKYWVYQGKPVMPDQFTFELLDFLHQLTHLSFSKMKALLERSHSPYYMLNRDRTLKNITETCKACAQVNASKSAVKQGTRVRGHRPGTHWEIDFTEIKPGLYGYKYLLVFIDTFSGWIEAFPTKKETAKVVTKKLLEEIFPRFGMPQVLGTDNGPAFVSKVSQTVADLLGIDWKLHCAYRPQSSGQVERMNRTIKETLTKLTLATGSRDWVLLLPLALYRARNTPGPHGLTPYEILYGAPPPLVNFPDPDMTRVTNSPSLQAHLQALYLVQHEVWRPLAAAYQEQLDRPVVPHPYRVGDTVWVRRHQTKNLEPRWKGPYTVLLTTPTALKVDGIAAWIHAAHVKAADPGGGPSSRLTWRVQRSQNPLKIRLTREAP

SEQ ID NO: 15 - DNA, 1767 bp, 3173 Thermostable Phage
atgggagaagatgggctatctttacctaagatgatgaatacaccaaaaccaattcttaaacctcaaccaaaagctttagtagaaccagtgctttgcgatagcattgatgaaataccagcgaaatataatgaaccagtatactttgacttggcaactgacgaagacagaccagttcttgcaagtatttatcaacctcactttgaacgcaaggtgtattgtttaaacctcttgaaagaaaaggtagcaaggtttaaagactggcttcttaaattctcagaaataagaggatggggtcttgactttgacttacgggttcttggctacacctacgaacaacttagaaacaagaagattgtagatgttcagcttgcgataaaagtccagcactacgagagatttaagcagggtgggaccaaaggtgaaggtttcagacttgatgatgtggcacgagatttgcttggtatagaatatccgatgaacaaaacaaaaattcgtgaaaccttcaaaaacaacatgtttcattcatttagcaacgaacaacttctttatgcctcgcttgatgcatacataccacacttgctttacgaacaactaacatcaagcacgcttaatagtcttgtttatcagcttgatcaacaggcacagaaagttgtgatagaaacatcgcaacacggcatgccagtaaaactaaaagcattagaagaagaaatacacagactaactcagctacgcagtgaaatgcaaaagcagataccatttaactataactctccaaaacaaacggcaaaattctttggagtaaatagttcttcaaaagatgtattgatggacttagctctacaaggaaatgaaatggctaaaaaggtgcttgaagcaagacaaatagaaaaatctcttgcttttgcaaaagacctctatgatatagctaaaagaagtggtggtagaatttacggcaacttctttactacaacagcaccatctggcagaatgtcttgctcggatataaatcttcaacagataccgcgtaggcttagatcattcataggctttgatacagaggacaaaaagcttatcaccgcagactttccgcaaattgagcttagacttgcaggtgtgatttggaatgaacctaaattcatagaagcatttaggcaaggtatagaccttcacaagcttacagcatcaatactgtttgataagaacatagaagaagtaagcaaggaagaaaggcaaattggaaaatctgcgaattttgggcttatctatggtattgcaccaaaaggtttcgcagaatattgtatagcgaacggtattaacatgacagaagagcaggcatacgaaatagtcagaaagtggaagaagtattacacaaagattgcagaacaacatcaagtagcatatgaaaggttcaaatacaatgagtatgtagataacgaaacatggcttaacagaacatatcgtgcatggaaaccacaagacctcttgaactatcaaatacaaggcagtggtgcggagctattcaagaaagctatagtattgttaaaagaaacaaagccagacttgaagatagtcaatctcgtgcatgatgagatagtagtagaagcagatagcaaagaagcacaagacttggctaagctaattaaagagaaaatggaggaagcgtgggattggtgtcttgaaaaagcagaagagtttggtaatagagttgctaaaataaaacttgaagtggaggagccacatgtgggtaatacatgggaaaagccttga

SEQ ID NO: 16 - protein, 588 aa, 3173 Thermostable Phage
MGEDGLSLPKMMNTPKPILKPQPKALVEPVLCDSIDEIPAKYNEPVYFDLETDEDRPVLASIYQPHFERKVYCLNLLKEKVARFKDWLLKFSEIRGWGLDFDLRVLGYTYEQLRNKKIVDVQLAIKVQHYERFKQGGTKGEGFRLDDVARDLLGIEYPMNKTKIRETFKNNMFHSFSNEQLLYASLDAYIPHLLYEQLTSSTLNSLVYQLDQQAQKVVIETSQHGMPVKLKALEEEIHRLTQLRSEMQKQIPFNYNSPKQTAKFFGVNSSSKDVLMDLALQGNEMAKKVLEARQIEKSLAFAKDLYDIAKRSGGRIYGNFFTTTAPSGRMSCSDINLQQIPRRLRSFIGFDTEDKKLITADFPQIELRLAGVIWNEPKFIEAFRQGIDLHKLTASILFDKNIEEVSKEERQIGKSANFGLIYGIAPKGFAEYCIANGINMTEEQAYEIVRKWKKYYTKIAEQHQVAYERFKYNEYVDNETWLNRTYRAWKPQDLLNYQIQGSGAELFKKAIVLLKETKPDLKIVNLVHDEIVVEADSKEAQDLAKLIKEKMEEAWDWCLEKAEEFGNRVAKIKLEVEEPHVGNTWEKP

SEQ ID NO: 17 - DNA, 1767 bp, 3173 Thermostable Phage; identical to SEQ ID NO: 15

SEQ ID NO: 18 - protein, 588 aa, 3173 Thermostable Phage
MGEDGLSLPKMMNTPKPILKPQPKALVEPVLCDSIDEIPAKYNEPVYFDLATDEDRPVLASIYQPHFERKVYCLNLLKEKVARFKDWLLKFSEIRGWGLDFDLRVLGYTYEQLRNKKIVDVQLAIKVQHYERFKQGGTKGEGFRLDDVARDLLGIEYPMNKTKIRETFKNNMFHSFSNEQLLYASLDAYIPHLLYEQLTSSTLNSLVYQLDQQAQKVVIETSQHGMPVKLKALEEEIHRLTQLRSEMQKQIPFNYNSPKQTAKFFGVNSSSKDVLMDLALQGNEMAKKVLEARQIEKSLAFAKDLYDIAKRSGGRIYGNFFTTTAPSGRMSCSDINLQQIPRRLRSFIGFDTEDKKLITADFPQIELRLAGVIWNEPKFIEAFRQGIDLHKLTASILFDKNIEEVSKEERQIGKSANFGLIYGIAPKGFAEYCIANGINMTEEQAYEIVRKWKKYYTKIAEQHQVAYERFKYNEYVDNETWLNRTYRAWKPQDLLNYQIQGSGAELFKKAIVLLKETKPDLKIVNLVHDEIVVEADSKEAQDLAKLIKEKMEEAWDWCLEKAEEFGNRVAKIKLEVEEPHVGNTWEKP

SEQ ID NO: 19 - DNA, 1767 bp, 3173 Thermostable Phage
atgggagaagatgggctatctttacctaagatgatgaatacaccaaaaccaattcttaaacctcaaccaaaagctttagtagaaccagtgctttgcgatagcattgatgaaataccagcgaaatataatgaaccagtatactttgccttggaaactgacgaagacagaccagttcttgcaagtatttatcaacctcactttgaacgcaaggtgtattgtttaaacctcttgaaagaaaaggtagcaaggtttaaagactggcttcttaaattctcagaaataagaggatggggtcttgactttgacttacgggttcttggctacacctacgaacaacttagaaacaagaagattgtagatgttcagcttgcgataaaagtccagcactacgagagatttaagcagggtgggaccaaaggtgaaggtttcagacttgatgatgtggcacgagatttgcttggtatagaatatccgatgaacaaaacaaaaattcgtgaaaccttcaaaaacaacatgtttcattcatttagcaacgaacaacttctttatgcctcgcttgatgcatacataccacacttgctttacgaacaactaacatcaagcacgcttaatagtcttgtttatcagcttgatcaacaggcacagaaagttgtgatagaaacatcgcaacacggcatgccagtaaaactaaaagcattagaagaagaaatacacagactaactcagctacgcagtgaaatgcaaaagcagataccatttaactataactctccaaaacaaacggcaaaattctttggagtaaatagttcttcaaaagatgtattgatggacttagctctacaaggaaatgaaatggctaaaaaggtgcttgaagcaagacaaatagaaaaatctcttgcttttgcaaaagacctctatgatatagctaaaagaagtggtggtagaatttacggcaacttctttactacaacagcaccatctggcagaatgtcttgctcggatataaatcttcaacagataccgcgtaggcttagatcattcataggctttgatacagaggacaaaaagcttatcaccgcagactttccgcaaattgagcttagacttgcaggtgtgatttggaatgaacctaaattcatagaagcatttaggcaaggtatagaccttcacaagcttacagcatcaatactgtttgataagaacatagaagaagtaagcaaggaagaaaggcaaattggaaaatctgcgaattatgggcttatctatggtattgcaccaaaaggtttcgcagaatattgtatagcgaacggtattaacatgacagaagagcaggcatacgaaatagtcagaaagtggaagaagtattacacaaagattgcagaacaacatcaagtagcatatgaaaggttcaaatacaatgagtatgtagataacgaaacatggcttaacagaacatatcgtgcatggaaaccacaagacctcttgaactatcaaatacaaggcagtggtgcggagctattcaagaaagctatagtattgttaaaagaaacaaagccagacttgaagatagtcaatctcgtgcatgatgagatagtagtagaagcagatagcaaagaagcacaagacttggctaagctaattaaagagaaaatggaggaagcgtgggattggtgtcttgaaaaagcagaagagtttggtaatagagttgctaaaataaaacttgaagtggaggagccacatgtgggtaatacatgggaaaagccttga

SEQ ID NO: 20 - protein, 588 aa, 3173 Thermostable Phage
MGEDGLSLPKMMNTPKPILKPQPKALVEPVLCDSIDEIPAKYNEPVYFALETDEDRPVLASIYQPHFERKVYCLNLLKEKVARFKDWLLKFSEIRGWGLDFDLRVLGYTYEQLRNKKIVDVQLAIKVQHYERFKQGGTKGEGFRLDDVARDLLGIEYPMNKTKIRETFKNNMFHSFSNEQLLYASLDAYIPHLLYEQLTSSTLNSLVYQLDQQAQKVVIETSQHGMPVKLKALEEEIHRLTQLRSEMQKQIPFNYNSPKQTAKFFGVNSSSKDVLMDLALQGNEMAKKVLEARQIEKSLAFAKDLYDIAKRSGGRIYGNFFTTTAPSGRMSCSDINLQQIPRRLRSFIGFDTEDKKLITADFPQIELRLAGVIWNEPKFIEAFRQGIDLHKLTASILFDKNIEEVSKEERQIGKSANYGLIYGIAPKGFAEYCIANGINMTEEQAYEIVRKWKKYYTKIAEQHQVAYERFKYNEYVDNETWLNRTYRAWKPQDLLNYQIQGSGAELFKKAIVLLKETKPDLKIVNLVHDEIVVEADSKEAQDLAKLIKEKMEEAWDWCLEKAEEFGNRVAKIKLEVEEPHVGNTWEKP

SEQ ID NO: 21 - DNA, 1725 bp, Dictyoglomus turgidus
atgttgaaaagatatgaattaaaaagcattcttcaaaaactttttcctgatcttgaagaaagggaaaatatagaaattaaagatgtaaaggaaatcaattttgaagaggcaaaaaaggaaggttgttttgcttttaaatgccttggagaaaaaggctttgaaggaatatccatctcctttaaggaaggagaaggatattttatagcttcctttgactttaatgatgaagttaaagggaaagttaaagatattatttctttcgaaaatattaaaaagattggagcttatatacagagggatctacattttctggactgtaaaataaaaggggaggtgtttgatgttagtctcgcatcctatcttttaaatccagaaagacaaaatcattcccttgacatacttataagagagtatttaaataggacctcttttattcctcaaaagtatgctgcttatctctttcctttaaaaactattctagaagaaaggataaaaaaggaagaattggaatttgtgctttttaatatagaaacaccgcttattcctgtactttactccatggaaaaatggggaataaaggtagataaggagtatttaaaaagtctctctgatgaattttgtgagagaattaagaaattggaagaggaaatatatgaacttgcaggtatgaagtttaatcttaattctccaaaacaactttctgaggttttatttgagagattgaagcttccttctggcaagaaaggaaaaacaggatattctacatcatctttggtgcttcaaaatttactgaatgctcatcctattgtgataaaaatcctccaatatagggagttatataaacttaaaagcacctatatagatgctattcctaatcttataaattcacaaacaggcagggttcatactaaatttaaccccacaggtacagccacaggaaggataagtagtagtgaacccaatctacaaaatattcccataaaaagcgaggaaggaagaaagataaggagagcctttatagcagatgatggatattattttgtatctcttgattattcccaaatagagcttagaattatggctcacctctctcaagaacctaaattaatatcagccttccaaaagggtgaagatattcatagaagaacagcagcagaaattttcggagtgcctgaagatgaagtagatgatcttttgaggtcgagggcaaaggcggttaactttggaattatttatggcatctcttcctttgggctttctgaaactgcaagtatcactccggaagaggctgaaaaatttatagattcatattttaaacattatccaagggtaaagctctttatagataaaactatttatgaggcaagagaaaagttatatgtaaagactttatttggaagaaaaagatatatacctgaaattagaagtataaataagcaggtgaggaatgcttatgaaaggatagctataaatgcgcctattcaaggaacagcggcggatataataaaacttgccatgatagagatttataaagaaatagaggaaaaaaatcttaagtcaagaatacttttacagattcacgatgaacttattcttgaagtgcctgaagaagaaatggagtttacccctttgatggcaaaggaaaagatggaaaaggttgtagaactttctgttcctcttgtggttgagatttcagtgggtaaaaatctggctgagctgaaatga

SEQ ID NO: 22 - protein, 573 aa, Dictyoglomus turgidus
MKRYELKSILQKLFPDLEERENIEIKDVKEINFEEAKKEGCFAFKCLGEKGFEGISISFKEGEGYFIASFDFNDEVKGKVKDIISFENIKKIGAYIQRDLHFLDCKIKGEVFDVSLASYLLNPERQNHSLDILIREYLNRTSFIPQKYAAYLFPLKTILEERIKKEELEFVLFNIETPLIPVLYSMEKWGIKVDKEYLKSLSDEFCERIKKLEEEIYELAGMKFNLNSPKQLSEVLFERLKLPSGKKGKTGYSTSSLVLQNLLNAHPIVIKILQYRELYKLKSTYIDAIPNLINSQTGRVHTKFNPTGTATGRISSSEPNLQNIPIKSEEGRKIRRAFIADDGYYFVSLDYSQIELRIMAHLSQEPKLISAFQKGEDIHRRTAAEIFGVPEDEVDDLLRSRAKAVNFGIIYGISSFGLSETASITPEEAEKFIDSYFKHYPRVKLFIDKTIYEAREKLYVKTLFGRKRYIPEIRSINKQVRNAYERIAINAPIQGTAADIIKLAMIEIYKEIEEKNLKSRILLQIHDELILEVPEEEMEFTPLMAKEKMEKVVELSVPLVVEISVGKNLAELK

SEQ ID NO: 23 - DNA, 2571 bp, Dictyoglomus thermophilum
atggagcagaaatctctgtgggatctttttcaagaaaataccgagaaagagtccaaaaggaagattctgattattgatggctcaagcctcatatacagggtttattacgcccttccccctttaaagacaaaaaatggtgaattaactaatgctctttatggcttcataagaatacttttaaaggccgtagaagattttaatcctgatcttgtaggcgttgcctttgatagacctgaacctacttttaggcatgtgatttataaagagtataaggctaagagaccacctatgaaggatgatttgaaagcgcagataccatggataagagaatttctaaggttaaatgatatacctctattggaagagcctggctatgaagcggatgatataatagctactatagtgaataaatataaggatgatttaaaatatattctctctggagatttagatcttttgcaattagtctcggacaaaacctttctaatacatcctcaaaagggaattactgagtttactatttatgatccaaaagctgtaaaggataggtttggagtagagccctataagattcccttatacaaagtattagtaggggacgaatctgataatattccaggagtaaatggaataggtcctaaaaaggcctcaaagattcttgagaaaatttcaagtgtagatgaatttaaaagtaaaataaaagttttggatagtgatttaagggagcttattgagaaaaattggaatattattgaaagaaatttagaacttgttactttaaaaaatatagataaggatcttattcttaaacccttcgagattaaaagagatgaaaaagtaatagattttttgaagagatatgaacttaagagtattcttcaaaagttatttcctgatcttcaagaggaagaaaatatagagattaaagatgtcgaagagatcaattttaatgaggtagaaaaagaaggctactttgcctttaaatgtcttggagatagggcttttgagggtatttctctttccttcaaggagggggaaggatattttatatctccttttgatttcaataatgagataagaaagaagattgaaaatataatttcttcagagaatgttaaaaaaattggctcttatattcaaagagatttacattttttaaactgtaaaataaagggcgatgtatttgatgttagtctcgcatcttatcttttgaaccctgaaagacaaaatcactctcttgatattttgataggagagtatctaaataaaacctcttttattcctcaaaaatacgctggttatctttttccgttaaagtctattcttgaggagaggataaagaatgaagggttagaatttgtactttataacatagagattccattaatccctgtactttactccatggagaagtgggggataaaggtagataaggaatatttaaaacagctttctgatgaattctgcgagagaattaaaaaattggaagaagagatatatgaacttgcaggaaccagatttaatctcaattctccaaaacaactttctgaagttttatttgagaggttaaaacttccttctggtaagaaaggaaaaacaggatattctacgtcgtcttctgtgcttcaaaacttaataaatgctcatcctatagtgagaaaaatcctccaatatagagaactctataaattgaagagtacttatgtggatgctattcctaatctggttaatccacaaacaggtagagttcatacaaaatttaatcctacaggtacagctacaggaagaataagtagtagtgaacctaatcttcagaatattcctataaaaagtgaagaaggtagaaagataagaagagccttcgtgtcagaagatggatattttcttgtatctcttgattattctcagatagagctaaggattatggctcatctttctcaggagcctaaattaatatctgccttccaaaaaggagaggatattcatagaagaacagcatcggagatttttggagtgccagaggaagaagttgatgatcttttaaggtcaagggcaaaggccgttaattttggaattatttatggtatctcttcttttggactttctgagactgtaagtattacaccagaagaggcagagaaatttatagactcgtattttaagcactatccaagagtgaagctttttatagataagactattcatgaggcaagagaaaaactgtacgttaaaaccttatttggcagaaaaagatatattcctgagattaagagcataaataaacaggtaaggaatgcctatgaaaggatagcaataaatgcgccaattcagggaacagctgctgatattataaaacttgccatgatagaaatttacaaggagattgaaaataaaaatctcaagtcaagaatactccttcaaattcatgatgagcttattcttgaagtgccagaggaggagatggaatttactcctttaatggcaaaggaaaaaatggaaaaggtggtagaactttcggttcctcttgtagttgaaatctcggtaggtaaaaatcttgctgaattaaaatga

SEQ ID NO: 24 - protein, 856 aa, Dictyoglomus thermophilum
MEQKSLWDLFQENTEKESKRKILIIDGSSLIYRVYYALPPLKTKNGELTNALYGFIRILLKAVEDFNPDLVGVAFDRPEPTFRHVIYKEYKAKRPPMKDDLKAQIPWIREFLRLNDIPLLEEPGYEADDIIATIVNKYKDDLKYILSGDLDLLQLVSDKTFLIHPQKGITEFTIYDPKAVKDRFGVEPYKIPLYKVLVGDESDNIPGVNGIGPKKASKILEKISSVDEFKSKIKVLDSDLRELIEKNWNIIERNLELVTLKNIDKDLILKPFEIKRDEKVIDFLKRYELKSILQKLFPDLQEEENIEIKDVEEINFNEVEKEGYFAFKCLGDRAFEGISLSFKEGEGYFISPFDFNNEIRKKIENIISSENVKKIGSYIQRDLHFLNCKIKGDVFDVSLASYLLNPERQNHSLDILIGEYLNKTSFIPQKYAGYLFPLKSILEERIKNEGLEFVLYNIEIPLIPVLYSMEKWGIKVDKEYLKQLSDEFCERIKKLEEEIYELAGTRFNLNSPKQLSEVLFERLKLPSGKKGKTGYSTSSSVLQNLINAHPIVRKILQYRELYKLKSTYVDAIPNLVNPQTGRVHTKFNPTGTATGRISSSEPNLQNIPIKSEEGRKIRRAFVSEDGYFLVSLDYSQIELRIMAHLSQEPKLISAFQKGEDIHRRTASEIFGVPEEEVDDLLRSRAKAVNFGIIYGISSFGLSETVSITPEEAEKFIDSYFKHYPRVKLFIDKTIHEAREKLYVKTLFGRKRYIPEIKSINKQVRNAYERIAINAPIQGTAADIIKLAMIEIYKEIENKNLKSRILLQIHDELILEVPEEEMEFTPLMAKEKMEKVVELSVPLVVEISVGKNLAELK

SEQ ID NO: 25 - DNA, 216 bp, Thermotoga maritime
atggcacgtggtaaagtgaaatggttcgactccaagaaaggttacggcttcattactaaagatgaaggtggcgatgtgttcgtgcactggtccgcgattgaaatggaaggcttcaagaccctgaaagaaggtcaagtggttgaattcgagattcaagaaggcaagaaaggtccgcaagcagcgcatgttaaagtggttgaaggatccgcgggttga

SEQ ID NO: 26 - protein, 67 aa, Thermotoga maritima
  RNA_BIND (14)..(18)
  RNA_BIND (26)..(30)
  DNA_BIND (32)..(35)
MARGKVKWFDSKKGYGFITKDEGGDVFVHWSAIEMEGFKTLKEGQVVEFEIQEGKKGPQAAHVKVVE

SEQ ID NO: 27 - DNA, 201 bp, Bacillus caldolyticus
atgcaacgtggtaaagtaaaatggtttaacaacgaaaaaggctacggtttcatcgaagtggagggcggttccgacgtattcgtccacttcacggcgatccaaggtgaagggttcaaaacgttagaagaaggccaagaagtttcgtttgaaatcgtccaaggaaaccgcggaccgcaagcagcgaacgttgtcaaattataa

SEQ ID NO: 28 - protein, 66 aa, Bacillus caldolyticus
  RNA_BIND (14)..(18)
  RNA_BIND (26)..(30)
  DNA_BIND (32)..(35)
MQRGKVKWFNNEKGYGFIEVEGGSDVFVHFTAIQGEGFKTLEEGQEVSFEIVQGNRGPQAANVVKL

SEQ ID NO: 29 - DNA, 213 bp, Escherichia coli
atgtccggtaaaatgactggtatcgtaaaatggttcaacgctgacaaaggcttcggcttcatcactcctgacgatggctctaaagatgtgttcgtacacttctctgctatccagaacgatggttacaaatctctggacgaaggtcagaaagtgtccttcaccatcgaaagcggcgctaaaggcccggcagctggtaacgtaaccagcctgtaa

SEQ ID NO: 30 - protein, 70 aa, Escherichia coli
  RNA_BIND (17)..(21)
  RNA_BIND (30)..(34)
  DNA_BIND (36)..(39)
MSGKMTGIVKWFNADKGFGFITPDDGSKDVFVHFSAIQNDGYKSLDEGQKVSFTIESGAKGPAAGNVTSL

SEQ ID NO: 31 - DNA, 195 bp, Sulfolobus solfataricus
atggcgacggttaaattcaagtataagggtgaggagaaagaagtggacatttccaagattaagaaagtgtggcgtgttggcaagatgatttcctttacctacgacgaaggtggtggtaagaccggtcgcggtgcggtttcggagaaagacgcaccaaaggagctgttgcaaatgttggagaaacaaaagaaatga

SEQ ID NO: 32 - protein, 64 aa, Sulfolobus solfataricus
  DNA_BIND (26)..(29)
MATVKFKYKGEEKEVDISKIKKVWRVGKMISFTYDEGGGKTGRGAVSEKDAPKELLQMLEKQKK

SEQ ID NO: 33 - DNA, 198 bp, Sulfolobus acidocaldarius
atggtgaaggttaaattcaagtataagggtgaggagctgcaagtggacacttccaagattaagaaagtgtggcgtgttggcaaggcgatttcctttacctacgaccaaggtaagaccggtcgcggtgcggtttcggagaaagacgcaccaaaggagctgttggacatgctggcacgtgcggaacgcgagaagaaatga

SEQ ID NO: 34 - protein, 66 aa, Sulfolobus acidocaldarius
  DNA_BIND (26)..(29)
MVKVKFKYKGEELQVDTSKIKKVWRVGKAVSFTYDDNGKTGRGAVSEKDAPKELLDMLARAEREKK

SEQ ID NO: 35 - DNA, 198 bp, Sulfolobus acidocaldarius; identical to SEQ ID NO: 33

SEQ ID NO: 36 - protein, 65 aa, Sulfolobus acidocaldarius
  DNA_BIND (26)..(29)
MVKVKFKYKGEELQVDTSKIKKVWRAGKAISFTYDQGKTGRGAVSEKDAPKELLDMLARAEREKK

SEQ ID NO: 37 - DNA, 183 bp, Sulfolobus shibatae
atgtcgtccggtaagaaagcggttaaagtgaagactccagctggtaaagaggctgagctggttcccgagaaagtttgggctctggctcccaaaggtcgtaaaggcgttaagataggtctgtttaaggatccagagactggtaaatactttcgtcataaactgccagatgactaccccatatga

SEQ ID NO: 38 - protein, 60 aa, Sulfolobus shibatae
  DNA_BIND (28)..(38)
MSSGKKAVKVKTPAGKEAELVPEKVWALAPKGRKGVKIGLFKDPETGKYFRHKLPDDYPI

SEQ ID NO: 39 - DNA, 801 bp, Thermus brockianus
atggcaagaggcctgaaccgcgtatacctcatcggctccctcacctcccggcccgacatgcgctacaccccgggggggctcgccatcctggagctcaacctggccgggcaggacaccctttgggacgagtccggccaggagcgggaactcccctggtaccaccgggtgcggcttctgggccgccaggcggagatgtggggggatgttttggagaagggccagctcctcttcgcggagggaaggctggaataccgccagtgggagcgggacggggagaagcggagcgagctccaggtgcgggccgacttcattgaccccttagacgcccgcgggcgggaaacccaggaggacgccaagagccagccccgcctccgccacgccctgaaccaggtggtcctcatgggcaacctcacccgcgacgccgagctccgctacaccccccaggggacggcggtggcccggctgggcctggcggtgaacgagcgccgccgggggccggggaccgaggaggaaaaaacccatttcatagaggttcaggcctggcgcgaactggccgagtgggccggggagctcaggaagggcgacgggcttttggtgatcggacgtttggtgaacgactcctggacgagctccagcggggaagggcgcttccagacccgcgtggaagccctccgcttggagcgacccacccgtgggcctgcccagaccggcggaagcaggccccaaccggtccagacgggtggggtggacattgacgagggactcgaggacttcccgccggaggaggatctgccgttttga

SEQ ID NO: 40 - protein, 266 aa, Thermus brockanus
MARGLNRVYLIGSLTSRPDMRYTPGGLAILELNLAGQDTLWDESGQERELPWYHRVRLLGRQAEMWGDVLEKGQLLFAEGRLEYRQWERDGEKRSELQVRADFIDPLDARGRETQEDAKSQPRLRHALNQVVLMGNLTRDAELRYTPQGTAVARLGLAVNERRRGPGTEEEKTHFIEVQAWRELAEWAGELRKGDGLLVIGRLVNDSWTSSSGEGRFQTRVEALRLERPTRGPAQTGGSRPQPVQTGGVDIDEGLEDFPPEEDLPF

SEQ ID NO: 41 - DNA, 1974 bp, Sulfolobus acidocaldarius
atggtgaaggttaaattcaagtataagggtgaggagctgcaagtggacacttccaagattaagaaagtgtggcgtgctggcaaggcgatttcctttacctacgaccaaggtaagaccggtcgcggtgcggtttcggagaaagacgcaccaaaggagctgttggacatgctggcacgtgcggaacgcgagaagaaaggatccgcgggtatgggagaagatgggctatctttacctaagatgatgaatacaccaaaaccaattcttaaacctcaaccaaaagctttagtagaaccagtgctttgcgatagcattgatgaaataccagcgaaatataatgaaccagtatactttgccttggaaactgacgaagacagaccagttcttgcaagtatttatcaacctcactttgaacgcaaggtgtattgtttaaacctcttgaaagaaaaggtagcaaggtttaaagactggcttcttaaattctcagaaataagaggatggggtcttgactttgacttacgggttcttggctacacctacgaacaacttagaaacaagaagattgtagatgttcagcttgcgataaaagtccagcactacgagagatttaagcagggtgggaccaaaggtgaaggtttcagacttgatgatgtggcacgagatttgcttggtatagaatatccgatgaacaaaacaaaaattcgtgaaaccttcaaaaacaacatgtttcattcatttagcaacgaacaacttctttatgcctcgcttgatgcatacataccacacttgctttacgaacaactaacatcaagcacgcttaatagtcttgtttatcagcttgatcaacaggcacagaaagttgtgatagaaacatcgcaacacggcatgccagtaaaactaaaagcattagaagaagaaatacacagactaactcagctacgcagtgaaatgcaaaagcagataccatttaactataactctccaaaacaaacggcaaaattctttggagtaaatagttcttcaaaagatgtattgatggacttagctctacaaggaaatgaaatggctaaaaaggtgcttgaagcaagacaaatagaaaaatctcttgcttttgcaaaagacctctatgatatagctaaaagaagtggtggtagaatttacggcaacttctttactacaacagcaccatctggcagaatgtcttgctcggatataaatcttcaacagataccgcgtaggcttagatcattcataggctttgatacagaggacaaaaagcttatcaccgcagactttccgcaaattgagcttagacttgcaggtgtgatttggaatgaacctaaattcatagaagcatttaggcaaggtatagaccttcacaagcttacagcatcaatactgtttgataagaacatagaagaagtaagcaaggaagaaaggcaaattggaaaatctgcgaattatgggcttatctatggtattgcaccaaaaggtttcgcagaatattgtatagcgaacggtattaacatgacagaagagcaggcatacgaaatagtcagaaagtggaagaagtattacacaaagattgcagaacaacatcaagtagcatatgaaaggttcaaatacaatgagtatgtagataacgaaacatggcttaacagaacatatcgtgcatggaaaccacaagacctcttgaactatcaaatacaaggcagtggtgcggagctattcaagaaagctatagtattgttaaaagaaacaaagccagacttgaagatagtcaatctcgtgcatgatgagatagtagtagaagcagatagcaaagaagcacaagacttggctaagctaattaaagagaaaatggaggaagcgtgggattggtgtcttgaaaaagcagaagagtttggtaatagagttgctaaaataaaacttgaagtggaggagccacatgtgggtaatacatgggaaaagccttga

SEQ ID NO: 42 - protein, 657 aa, Sulfolobus acidocaldarius
  DNA_BIND (26)..(29)
  Linker (66)..(69)
MVKVKFKYKGEELQVDTSKIKKVWRAGKAISFTYDQGKTGRGAVSEKDAPKELLDMLARAEREKKGSAGMGEDGLSLPKMMNTPKPILKPQPKALVEPVLCDSIDEIPAKYNEPVYFALETDEDRPVLASIYQPHFERKVYCLNLLKEKVARFKDWLLKFSEIRGWGLDFDLRVLGYTYEQLRNKKIVDVQLAIKVQHYERFKQGGTKGEGFRLDDVARDLLGIEYPMNKTKIRETFKNNMFHSFSNEQLLYASLDAYIPHLLYEQLTSSTLNSLVYQLDQQAQKVVIETSQHGMPVKLKALEEEIHRLTQLRSEMQKQIPFNYNSPKQTAKFFGVNSSSKDVLMDLALQGNEMAKKVLEARQIEKSLAFAKDLYDIAKRSGGRIYGNFFTTTAPSGRMSCSDINLQQIPRRLRSFIGFDTEDKKLITADFPQIELRLAGVIWNEPKFIEAFRQGIDLHKLTASILFDKNIEEVSKEERQIGKSANYGLIYGIAPKGFAEYCIANGINMTEEQAYEIVRKWKKYYTKIAEQHQVAYERFKYNEYVDNETWLNRTYRAWKPQDLLNYQIQGSGAELFKKAIVLLKETKPDLKIVNLVHDEIVVEADSKEAQDLAKLIKEKMEEAWDWCLEKAEEFGNRVAKIKLEVEEPHVGNTWEKP

SEQ ID NO: 43 - DNA, 1974 bp, Sulfolobus acidocaldarius
atggtgaaggttaaattcaagtataagggtgaggagctgcaagtggacacttccaagattaagaaagtgtggcgtgttggcaaggcgatttcctttacctacgaccaaggtaagaccggtcgcggtgcggtttcggagaaagacgcaccaaaggagctgttggacatgctggcacgtgcggaacgcgagaagaaaggatccgcgggtatgggagaagatgggctatctttacctaagatgatgaatacaccaaaaccaattcttaaacctcaaccaaaagctttagtagaaccagtgctttgcgatagcattgatgaaataccagcgaaatataatgaaccagtatactttgccttggaaactgacgaagacagaccagttcttgcaagtatttatcaacctcactttgaacgcaaggtgtattgtttaaacctcttgaaagaaaaggtagcaaggtttaaagactggcttcttaaattctcagaaataagaggatggggtcttgactttgacttacgggttcttggctacacctacgaacaacttagaaacaagaagattgtagatgttcagcttgcgataaaagtccagcactacgagagatttaagcagggtgggaccaaaggtgaaggtttcagacttgatgatgtggcacgagatttgcttggtatagaatatccgatgaacaaaacaaaaattcgtgaaaccttcaaaaacaacatgtttcattcatttagcaacgaacaacttctttatgcctcgcttgatgcatacataccacacttgctttacgaacaactaacatcaagcacgcttaatagtcttgtttatcagcttgatcaacaggcacagaaagttgtgatagaaacatcgcaacacggcatgccagtaaaactaaaagcattagaagaagaaatacacagactaactcagctacgcagtgaaatgcaaaagcagataccatttaactataactctccaaaacaaacggcaaaattctttggagtaaatagttcttcaaaagatgtattgatggacttagctctacaaggaaatgaaatggctaaaaaggtgcttgaagcaagacaaatagaaaaatctcttgcttttgcaaaagacctctatgatatagctaaaagaagtggtggtagaatttacggcaacttctttactacaacagcaccatctggcagaatgtcttgctcggatataaatcttcaacagataccgcgtaggcttagatcattcataggctttgatacagaggacaaaaagcttatcaccgcagactttccgcaaattgagcttagacttgcaggtgtgatttggaatgaacctaaattcatagaagcatttaggcaaggtatagaccttcacaagcttacagcatcaatactgtttgataagaacatagaagaagtaagcaaggaagaaaggcaaattggaaaatctgcgaattatgggcttatctatggtattgcaccaaaaggtttcgcagaatattgtatagcgaacggtattaacatgacagaagagcaggcatacgaaatagtcagaaagtggaagaagtattacacaaagattgcagaacaacatcaagtagcatatgaaaggttcaaatacaatgagtatgtagataacgaaacatggcttaacagaacatatcgtgcatggaaaccacaagacctcttgaactatcaaatacaaggcagtggtgcggagctattcaagaaagctatagtattgttaaaagaaacaaagccagacttgaagatagtcaatctcgtgcatgatgagatagtagtagaagcagatagcaaagaagcacaagacttggctaagctaattaaagagaaaatggaggaagcgtgggattggtgtcttgaaaaagcagaagagtttggtaatagagttgctaaaataaaacttgaagtggaggagccacatgtgggtaatacatgggaaaagccttga

SEQ ID NO: 44 - protein, 657 aa, Sulfolobus acidocaldarius
  DNA_BIND (26)..(29)
  Linker (66)..(69)
MVKVKFKYKGEELQVDTSKIKKVWRVGKAISFTYDQGKTGRGAVSEKDAPKELLDMLARAEREKKGSAGMGEDGLSLPKMMNTPKPILKPQPKALVEPVLCDSIDEIPAKYNEPVYFALETDEDRPVLASIYQPHFERKVYCLNLLKEKVARFKDWLLKFSEIRGWGLDFDLRVLGYTYEQLRNKKIVDVQLAIKVQHYERFKQGGTKGEGFRLDDVARDLLGIEYPMNKTKIRETFKNNMFHSFSNEQLLYASLDAYIPHLLYEQLTSSTLNSLVYQLDQQAQKVVIETSQHGMPVKLKALEEEIHRLTQLRSEMQKQIPFNYNSPKQTAKFFGVNSSSKDVLMDLALQGNEMAKKVLEARQIEKSLAFAKDLYDIAKRSGGRIYGNFFTTTAPSGRMSCSDINLQQIPRRLRSFIGFDTEDKKLITADFPQIELRLAGVIWNEPKFIEAFRQGIDLHKLTASILFDKNIEEVSKEERQIGKSANYGLIYGIAPKGFAEYCIANGINMTEEQAYEIVRKWKKYYTKIAEQHQVAYERFKYNEYVDNETWLNRTYRAWKPQDLLNYQIQGSGAELFKKAIVLLKETKPDLKIVNLVHDEIVVEADSKEAQDLAKLIKEKMEEAWDWCLEKAEEFGNRVAKIKLEVEEPHVGNTWEKP

SEQ ID NO: 45 - DNA, 1980 bp, Thermotoga maritima
atggcacgtggtaaagtgaaatggttcgactccaagaaaggttacggcttcattactaaagatgaaggtggcgatgtgttcgtgcactggtccgcgattgaaatggaaggcttcaagaccctgaaagaaggtcaagtggttgaattcgagattcaagaaggcaagaaaggtccgcaagcagcgcatgttaaagtggttgaaggatccgcgggtatgggagaagatgggctatctttacctaagatgatgaatacaccaaaaccaattcttaaacctcaaccaaaagctttagtagaaccagtgctttgcgatagcattgatgaaataccagcgaaatataatgaaccagtatactttgccttggaaactgacgaagacagaccagttcttgcaagtatttatcaacctcactttgaacgcaaggtgtattgtttaaacctcttgaaagaaaaggtagcaaggtttaaagactggcttcttaaattctcagaaataagaggatggggtcttgactttgacttacgggttcttggctacacctacgaacaacttagaaacaagaagattgtagatgttcagcttgcgataaaagtccagcactacgagagatttaagcagggtgggaccaaaggtgaaggtttcagacttgatgatgtggcacgagatttgcttggtatagaatatccgatgaacaaaacaaaaattcgtgaaaccttcaaaaacaacatgtttcattcatttagcaacgaacaacttctttatgcctcgcttgatgcatacataccacacttgctttacgaacaactaacatcaagcacgcttaatagtcttgtttatcagcttgatcaacaggcacagaaagttgtgatagaaacatcgcaacacggcatgccagtaaaactaaaagcattagaagaagaaatacacagactaactcagctacgcagtgaaatgcaaaagcagataccatttaactataactctccaaaacaaacggcaaaattctttggagtaaatagttcttcaaaagatgtattgatggacttagctctacaaggaaatgaaatggctaaaaaggtgcttgaagcaagacaaatagaaaaatctcttgcttttgcaaaagacctctatgatatagctaaaagaagtggtggtagaatttacggcaacttctttactacaacagcaccatctggcagaatgtcttgctcggatataaatcttcaacagataccgcgtaggcttagatcattcataggctttgatacagaggacaaaaagcttatcaccgcagactttccgcaaattgagcttagacttgcaggtgtgatttggaatgaacctaaattcatagaagcatttaggcaaggtatagaccttcacaagcttacagcatcaatactgtttgataagaacatagaagaagtaagcaaggaagaaaggcaaattggaaaatctgcgaattatgggcttatctatggtattgcaccaaaaggtttcgcagaatattgtatagcgaacggtattaacatgacagaagagcaggcatacgaaatagtcagaaagtggaagaagtattacacaaagattgcagaacaacatcaagtagcatatgaaaggttcaaatacaatgagtatgtagataacgaaacatggcttaacagaacatatcgtgcatggaaaccacaagacctcttgaactatcaaatacaaggcagtggtgcggagctattcaagaaagctatagtattgttaaaagaaacaaagccagacttgaagatagtcaatctcgtgcatgatgagatagtagtagaagcagatagcaaagaagcacaagacttggctaagctaattaaagagaaaatggaggaagcgtgggattggtgtcttgaaaaagcagaagagtttggtaatagagttgctaaaataaaacttgaagtggaggagccacatgtgggtaatacatgggaaaagccttga

SEQ ID NO: 46 - protein, 659 aa, Thermotoga maritima
  RNA_BIND (14)..(18)
  RNA_BIND (26)..(30)
  DNA_BIND (32)..(35)
  Linker (68)..(71)
MARGKVKWFDSKKGYGFITKDEGGDVFVHWSAIEMEGFKTLKEGQVVEFEIQEGKKGPQAAHVKVVEGSAGMGEDGLSLPKMMNTPKPILKPQPKALVEPVLCDSIDEIPAKYNEPVYFALETDEDRPVLASIYQPHFERKVYCLNLLKEKVARFKDWLLKFSEIRGWGLDFDLRVLGYTYEQLRNKKIVDVQLAIKVQHYERFKQGGTKGEGFRLDDVARDLLGIEYPMNKTKIRETFKNNMFHSFSNEQLLYASLDAYIPHLLYEQLTSSTLNSLVYQLDQQAQKVVIETSQHGMPVKLKALEEEIHRLTQLRSEMQKQIPFNYNSPKQTAKFFGVNSSSKDVLMDLALQGNEMAKKVLEARQIEKSLAFAKDLYDIAKRSGGRIYGNFFTTTAPSGRMSCSDINLQQIPRRLRSFIGFDTEDKKLITADFPQIELRLAGVIWNEPKFIEAFRQGIDLHKLTASILFDKNIEEVSKEERQIGKSANYGLIYGIAPKGFAEYCIANGINMTEEQAYEIVRKWKKYYTKIAEQHQVAYERFKYNEYVDNETWLNRTYRAWKPQDLLNYQIQGSGAELFKKAIVLLKETKPDLKIVNLVHDEIVVEADSKEAQDLAKLIKEKMEEAWDWCLEKAEEFGNRVAKIKLEVEEPHVGNTWEKP

SEQ ID NO: 47 - DNA, 1974 bp, Sulfolobus acidocaldarius
atggtgaaggttaaattcaagtataagggtgaggagctgcaagtggacacttccaagattaagaaagtgtggcgtgttggcaaggcgatttcctttacctacgaccaaggtaagaccggtcgcggtgcggtttcggagaaagacgcaccaaaggagctgttggacatgctggcacgtgcggaacgcgagaagaaaggatccgcgggtatgggagaagatgggctatctttacctaagatgatgaatacaccaaaaccaattcttaaacctcaaccaaaagctttagtagaaccagtgctttgcgatagcattgatgaaataccagcgaaatataatgaaccagtatactttgccttggaaactgacgaagacagaccagttcttgcaagtatttatcaacctcactttgaacgcaaggtgtattgtttaaacctcttgaaagaaaaggtagcaaggtttaaagactggcttcttaaattctcagaaataagaggatggggtcttgactttgacttacgggttcttggctacacctacgaacaacttagaaacaagaagattgtagatgttcagcttgcgataaaagtccagcactacgagagatttaagcagggtgggaccaaaggtgaaggtttcagacttgatgatgtggcacgagatttgcttggtatagaatatccgatgaacaaaacaaaaattcgtgaaaccttcaaaaacaacatgtttcattcatttagcaacgaacaacttctttatgcctcgcttgatgcatacataccacacttgctttacgaacaactaacatcaagcacgcttaatagtcttgtttatcagcttgatcaacaggcacagaaagttgtgatagaaacatcgcaacacggcatgccagtaaaactaaaagcattagaagaagaaatacacagactaactcagctacgcagtgaaatgcaaaagcagataccatttaactataactctccaaaacaaacggcaaaattctttggagtaaatagttcttcaaaagatgtattgatggacttagctctacaaggaaatgaaatggctaaaaaggtgcttgaagcaagacaaatagaaaaatctcttgcttttgcaaaagacctctatgatatagctaaaagaagtggtggtagaatttacggcaacttctttactacaacagcaccatctggcagaatgtcttgctcggatataaatcttcaacagataccgcgtaggcttagatcattcataggctttgatacagaggacaaaaagcttatcaccgcagactttccgcaaattgagcttagacttgcaggtgtgatttggaatgaacctaaattcatagaagcatttaggcaaggtatagaccttcacaagcttacagcatcaatactgtttgataagaacatagaagaagtaagcaaggaagaaaggcaaattggaaaatctgcgaattttgggcttatctatggtattgcaccaaaaggtttcgcagaatattgtatagcgaacggtattaacatgacagaagagcaggcatacgaaatagtcagaaagtggaagaagtattacacaaagattgcagaacaacatcaagtagcatatgaaaggttcaaatacaatgagtatgtagataacgaaacatggcttaacagaacatatcgtgcatggaaaccacaagacctcttgaactatcaaatacaaggcagtggtgcggagctattcaagaaagctatagtattgttaaaagaaacaaagccagacttgaagatagtcaatctcgtgcatgatgagatagtagtagaagcagatagcaaagaagcacaagacttggctaagctaattaaagagaaaatggaggaagcgtgggattggtgtcttgaaaaagcagaagagtttggtaatagagttgctaaaataaaacttgaagtggaggagccacatgtgggtaatacatgggaaaagccttga

SEQ ID NO: 48 - protein, 657 aa, Sulfolobus acidocaldarius
  DNA_BIND (26)..(29)
  Linker (66)..(69)
MVKVKFKYKGEELQVDTSKIKKVWRVGKAISFTYDQGKTGRGAVSEKDAPKELLDMLARAEREKKGSAGMGEDGLSLPKMMNTPKPILKPQPKALVEPVLCDSIDEIPAKYNEPVYFALETDEDRPVLASIYQPHFERKVYCLNLLKEKVARFKDWLLKFSEIRGWGLDFDLRVLGYTYEQLRNKKIVDVQLAIKVQHYERFKQGGTKGEGFRLDDVARDLLGIEYPMNKTKIRETFKNNMFHSFSNEQLLYASLDAYIPHLLYEQLTSSTLNSLVYQLDQQAQKVVIETSQHGMPVKLKALEEEIHRLTQLRSEMQKQIPFNYNSPKQTAKFFGVNSSSKDVLMDLALQGNEMAKKVLEARQIEKSLAFAKDLYDIAKRSGGRIYGNFFTTTAPSGRMSCSDINLQQIPRRLRSFIGFDTEDKKLITADFPQIELRLAGVIWNEPKFIEAFRQGIDLHKLTASILFDKNIEEVSKEERQIGKSANFGLIYGIAPKGFAEYCIANGINMTEEQAYEIVRKWKKYYTKIAEQHQVAYERFKYNEYVDNETWLNRTYRAWKPQDLLNYQIQGSGAELFKKAIVLLKETKPDLKIVNLVHDEIVVEADSKEAQDLAKLIKEKMEEAWDWCLEKAEEFGNRVAKIKLEVEEPHVGNTWEKP

SEQ ID NO: 49 - DNA, 1974 bp, Sulfolobus acidocaldarius
atggtgaaggttaaattcaagtataagggtgaggagctgcaagtggacacttccaagattaagaaagtgtggcgtgttggcaaggcgatttcctttacctacgaccaaggtaagaccggtcgcggtgcggtttcggagaaagacgcaccaaaggagctgttggacatgctggcacgtgcggaacgcgagaagaaaggatccgcgggtatgggagaagatgggctatctttacctaagatgatgaatacaccaaaaccaattcttaaacctcaaccaaaagctttagtagaaccagtgctttgcgatagcattgatgaaataccagcgaaatataatgaaccagtatactttgacttggaaactgacgaagacagaccagttcttgcaagtatttatcaacctcactttgaacgcaaggtgtattgtttaaacctcttgaaagaaaaggtagcaaggtttaaagactggcttcttaaattctcagaaataagaggatggggtcttgactttgacttacgggttcttggctacacctacgaacaacttagaaacaagaagattgtagatgttcagcttgcgataaaagtccagcactacgagagatttaagcagggtgggaccaaaggtgaaggtttcagacttgatgatgtggcacgagatttgcttggtatagaatatccgatgaacaaaacaaaaattcgtgaaaccttcaaaaacaacatgtttcattcatttagcaacgaacaacttctttatgcctcgcttgatgcatacataccacacttgctttacgaacaactaacatcaagcacgcttaatagtcttgtttatcagcttgatcaacaggcacagaaagttgtgatagaaacatcgcaacacggcatgccagtaaaactaaaagcattagaagaagaaatacacagactaactcagctacgcagtgaaatgcaaaagcagataccatttaactataactctccaaaacaaacggcaaaattctttggagtaaatagttcttcaaaagatgtattgatggacttagctctacaaggaaatgaaatggctaaaaaggtgcttgaagcaagacaaatagaaaaatctcttgcttttgcaaaagacctctatgatatagctaaaagaagtggtggtagaatttacggcaacttctttactacaacagcaccatctggcagaatgtcttgctcggatataaatcttcaacagataccgcgtaggcttagatcattcataggctttgatacagaggacaaaaagcttatcaccgcagactttccgcaaattgagcttagacttgcaggtgtgatttggaatgaacctaaattcatagaagcatttaggcaaggtatagaccttcacaagcttacagcatcaatactgtttgataagaacatagaagaagtaagcaaggaagaaaggcaaattggaaaatctgcgaattttgggcttatctatggtattgcaccaaaaggtttcgcagaatattgtatagcgaacggtattaacatgacagaagagcaggcatacgaaatagtcagaaagtggaagaagtattacacaaagattgcagaacaacatcaagtagcatatgaaaggttcaaatacaatgagtatgtagataacgaaacatggcttaacagaacatatcgtgcatggaaaccacaagacctcttgaactatcaaatacaaggcagtggtgcggagctattcaagaaagctatagtattgttaaaagaaacaaagccagacttgaagatagtcaatctcgtgcatgatgagatagtagtagaagcagatagcaaagaagcacaagacttggctaagctaattaaagagaaaatggaggaagcgtgggattggtgtcttgaaaaagcagaagagtttggtaatagagttgctaaaataaaacttgaagtggaggagccacatgtgggtaatacatgggaaaagccttga

SEQ ID NO: 50 - protein, 657 aa, Sulfolobus acidocaldarius
  DNA_BIND (26)..(29)
  Linker (66)..(69)
MVKVKFKYKGEELQVDTSKIKKVWRVGKAISFTYDQGKTGRGAVSEKDAPKELLDMLARAEREKKGSAGMGEDGLSLPKMMNTPKPILKPQPKALVEPVLCDSIDEIPAKYNEPVYFDLETDEDRPVLASIYQPHFERKVYCLNLLKEKVARFKDWLLKFSEIRGWGLDFDLRVLGYTYEQLRNKKIVDVQLAIKVQHYERFKQGGTKGEGFRLDDVARDLLGIEYPMNKTKIRETFKNNMFHSFSNEQLLYASLDAYIPHLLYEQLTSSTLNSLVYQLDQQAQKVVIETSQHGMPVKLKALEEEIHRLTQLRSEMQKQIPFNYNSPKQTAKFFGVNSSSKDVLMDLALQGNEMAKKVLEARQIEKSLAFAKDLYDIAKRSGGRIYGNFFTTTAPSGRMSCSDINLQQIPRRLRSFIGFDTEDKKLITADFPQIELRLAGVIWNEPKFIEAFRQGIDLHKLTASILFDKNIEEVSKEERQIGKSANFGLIYGIAPKGFAEYCIANGINMTEEQAYEIVRKWKKYYTKIAEQHQVAYERFKYNEYVDNETWLNRTYRAWKPQDLLNYQIQGSGAELFKKAIVLLKETKPDLKIVNLVHDEIVVEADSKEAQDLAKLIKEKMEEAWDWCLEKAEEFGNRVAKIKLEVEEPHVGNTWEKP

SEQ ID NO: 51 - DNA, 1839 bp, Thermus aquaticus
atggcgacggttaaattcaagtataagggtgaggagaaagaagtggacatttccaagattaagaaagtgtggcgtgttggcaagatgatttcctttacctacgacgaaggtggtggtaagaccggtcgcggtgcggtttcggagaaagacgcaccaaaggagctgttgcaaatgttggagaaacaaaagaaaggatccgcgggtatgagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgttcggcgtcccccgggaggccgtggaccccctgatgcgccgggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtga

SEQ ID NO: 52 - protein, 612 aa, Thermus aquaticus
  DNA_BIND (26)..(29)
  Linker (65)..(68)
MATVKFKYKGEEKEVDISKIKKVWRVGKMISFTYDEGGGKTGRGAVSEKDAPKELLQMLEKQKKGSAGMSPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 53 - DNA, 2940 bp, Sulfolobus acidocaldarius
atggcgcaccatcatcaccatcacgaaaacctgtactttcagggtgcgacggttaaattcaagtataagggtgaggagaaagaagtggacatttccaagattaagaaagtgtggcgtgttggcaagatgatttcctttacctacgacgaaggtggtggtaagaccggtcgcggtgcggtttcggagaaagacgcaccaaaggagctgttgcaaatgttggagaaacaaaagaaaggctccgcgggtaaagaattttatatctctattgaaacagtcggaaataacattgttgaacgttatattgatgaaaatggaaaggaacgtacccgtgaagtagaatatcttccaactatgtttaggcattgtaaggaagagtcaaaatacaaagacatctatggtaaaaactgcgctcctcaaaaatttccatcaatgaaagatgctcgagattggatgaagcgaatggaagacatcggtctcgaagctctcggtatgaacgattttaaactcgcttatataagtgatacatatggttcagaaattgtttatgaccgaaaatttgttcgtgtagctaactgtgacattgaggttactggtgataaatttcctgacccaatgaaagcagaatatgaaattgatgctatcactcattacgattcaattgacgatcgtttttatgttttcgaccttttgaattcaatgtacggttcagtatcaaaatgggatgcaaagttagctgctaagcttgactgtgaaggtggtgatgaagttcctcaagaaattcttgaccgagtaatttatatgccattcgataatgagcgtgatatgctcatggaatatatcaatctttgggaacagaaacgacctgctatttttactggttggaatattgaggggtttgccgttccgtatatcatgaatcgtgttaaaatgattctgggtgaacgtagtatgaaacgtttctctccaatcggtcgggtaaaatctaaactaattcaaaatatgtacggtagcaaagaaatttattctattgatggcgtatctattcttgattatttagatttgtacaagaaattcgcttttactaatttgccgtcattctctttggaatcagttgctcaacatgaaaccaaaaaaggtaaattaccatacgacggtcctattaataaacttcgtgagactaatcatcaacgatacattagttataacatcattgacgtagaatcagttcaagcaatcgataaaattcgtgggtttatcgatctagttttaagtatgtcttattacgctaaaatgcctttttctggtgtaatgagtcctattaaaacttgggatgctattatttttaactcattgaaaggtgaacataaggttattcctcaacaaggttcgcacgttaaacagagttttccgggtgcatttgtgtttgaacctaaaccaattgcacgtcgatacattatgagttttgacttgacgtctctgtatccgagcattattcgccaggttaacattagtcctgaaactattcgtggtcagtttaaagttcatccaattcatgaatatatcgcaggaacagctcctaaaccgagtgatgaatattcttgttctccgaatggatggatgtatgataaacatcaagaaggtatcattccaaaggaaatcgctaaagtatttttccagcgtaaagactggaaaaagaaaatgttcgctgaagaaatgaatgccgaagctattaaaaagattattatgaaaggcgcagggtcttgttcaactaaaccagaagttgaacgatatgttaagttcagtgatgatttcttaaatgaactatcgaattacaccgaatctgttctcaatagtctgattgaagaatgtgaaaaagcagctacacttgctaatacaaatcagctgaaccgtaaaattctcattaacagtctttatggtgctcttggtaatattcatttccgttactatgatttgcgaaatgctactgctatcacaattttcggccaagtcggtattcagtggattgctcgtaaaattaatgaatatctgaataaagtatgcggaactaatgatgaagatttcattgcagcaggtgatactgattcggtatatgtttgcgtagataaagttattgaaaaagttggtcttgaccgattcaaagagcagaacgatttggttgaattcatgaatcagttcggtaagaaaaagatggaacctatgattgatgttgcatatcgtgagttatgtgattatatgaataaccgcgagcatctgatgcatatggaccgtgaagctatttcttgccctccgcttggttcaaagggcgttggtggattttggaaagcgaaaaagcgttatgctctgaacgtttatgatatggaagataagcgatttgctgaaccgcatctaaaaatcatgggtatggaaactcagcagagttcaacaccaaaagcagtgcaagaagctctcgaagaaagtattcgtcgtattcttcaggaaggtgaagagtctgtccaagaatactacaagaacttcgagaaagaatatcgtcaacttgactataaagttattgctgaagtaaaaactgcgaacgatatagcgaaatatgatgataaaggttggccaggatttaaatgcccgttccatattcgtggtgtgctaacttatcgtcgagctgttagcggtttaggtgtagctccaattttggatggaaataaagtaatggttcttccattacgtgaaggaaatccatttggtgacaagtgcattgcttggccatcgggtacagaacttccaaaagaaattcgttctgatgtgctatcttggattgaccactcaactttgttccaaaaatcgtttgttaaaccgcttgcgggtatgtgtgaatcggctggcatggactatgaagaaaaagcttcgttagacttcctgtttggctga

SEQ ID NO: 54 - protein, 972 aa, Sulfolobus acidocaldarius
  DNA_BIND (32)..(35)
  Linker (72)..(75)
MVHHHHHHKVKFKYKGEELQVDTSKIKKVWRVGKAISFTYDQGKTGRGAVSEKDAPKELLDMLARAEREKKGSAGKEFYISIETVGNNIVERYIDENGKERTREVEYLPTMFRHCKEESKYKDIYGKNCAPQKFPSMKDARDWMKRMEDIGLEALGMNDFKLAYISDTYGSEIVYDRKFVRVANCDIEVTGDKFPDPMKAEYEIDAITHYDSIDDRFYVFDLLNSMYGSVSKWDAKLAAKLDCEGGDEVPQEILDRVIYMPFDNERDMLMEYINLWEQKRPAIFTGWNIEGFAVPYIMNRVKMILGERSMKRFSPIGRVKSKLIQNMYGSKEIYSIDGVSILDYLDLYKKFAFTNLPSFSLESVAQHETKKGKLPYDGPINKLRETNHQRYISYNIIDVESVQAIDKIRGFIDLVLSMSYYAKMPFSGVMSPIKTWDAIIFNSLKGEHKVIPQQGSHVKQSFPGAFVFEPKPIARRYIMSFDLTSLYPSIIRQVNISPETIRGQFKVHPIHEYIAGTAPKPSDEYSCSPNGWMYDKHQEGIIPKEIAKVFFQRKDWKKKMFAEEMNAEAIKKIIMKGAGSCSTKPEVERYVKFSDDFLNELSNYTESVLNSLIEECEKAATLANTNQLNRKILINSLYGALGNIHFRYYDLRNATAITIFGQVGIQWIARKINEYLNKVCGTNDEDFIAAGDTDSVYVCVDKVIEKVGLDRFKEQNDLVEFMNQFGKKKMEPMIDVAYRELCDYMNNREHLMHMDREAISCPPLGSKGVGGFWKAKKRYALNVYDMEDKRFAEPHLKIMGMETQQSSTPKAVQEALEESIRRILQEGEESVQEYYKNFEKEYRQLDYKVIAEVKTANDIAKYDDKGWPGFKCPFHIRGVLTYRRAVSGLGVAPILDGNKVMVLPLREGNPFGDKCIAWPSGTELPKEIRSDVLSWIDHSTLFQKSFVKPLAGMCESAGMDYEEKASLDFLFG

SEQ ID NO: 55 - DNA, 2046 bp, Sulfolobus acidocaldarius
atggtgcatcaccatcaccatcataaggttaaattcaagtataagggtgaggagctgcaagtggacacttccaagattaagaaagtgtggcgtgttggcaaggcgatttcctttacctacgaccaaggtaagaccggtcgcggtgcggtttcggagaaagacgcaccaaaggagctgttggacatgctggcacgtgcggaacgcgagaagaaaggatccgcgggtatggtgatttcttatgacaactacgtcaccatccttgatgaagaaacactgaaagcgtggattgcgaagctggaaaaagcgccggtatttgcatttgctaccgcaaccgacagccttgataacatctctgctaacctggtcgggctttcttttgctatcgagccaggcgtagcggcatatattccggttgctcatgattatcttgatgcgcccgatcaaatctctcgcgagcgtgcactcgagttgctaaaaccgctgctggaagatgaaaaggcgctgaaggtcgggcaaaacctgaaatacgatcgcggtattctggcgaactacggcattgaactgcgtgggattgcgtttgataccatgctggagtcctacattctcaatagcgttgccgggcgtcacgatatggacagcctcgcggaacgttggttgaagcacaaaaccatcacttttgaagagattgctggtaaaggcaaaaatcaactgacctttaaccagattgccctcgaagaagccggacgttacgccgccgaagatgcagatgtcaccttgcagttgcatctgaaaatgtggccggatctgcaaaaacacaaagggccgttgaacgtcttcgagaatatcgaaatgccgctggtgccggtgctttcacgcattgaacgtaacggtgtgaagatcgatccgaaagtgctgcacaatcattctgaagagctcacccttcgtctggctgagctggaaaagaaagcgcatgaaattgcaggtgaggaatttaacctttcttccaccaagcagttacaaaccattctctttgaaaaacagggcattaaaccgctgaagaaaacgccgggtggcgcgccgtcaacgtcggaagaggtactggaagaactggcgctggactatccgttgccaaaagtgattctggagtatcgtggtctggcgaagctgaaatcgacctacaccgacaagctgccgctgatgatcaacccgaaaaccgggcgtgtgcatacctcttatcaccaggcagtaactgcaacgggacgtttatcgtcaaccgatcctaacctgcaaaacattccggtgcgtaacgaagaaggtcgtcgtatccgccaggcgtttattgcgccagaggattatgtgattgtctcagcggactactcgcagattgaactgcgcattatggcgcatctttcgcgtgacaaaggcttgctgaccgcattcgcggaaggaaaagatatccaccgggcaacggcggcagaagtgtttggtttgccactggaaaccgtcaccagcgagcaacgccgtagcgcgaaagcgatcaactttggtctgatttatggcatgagtgctttcggtctggcgcggcaattgaacattccacgtaaagaagcgcagaagtacatggacctttacttcgaacgctaccctggcgtgctggagtatatggaacgcacccgtgctcaggcgaaagagcagggctacgttgaaacgctggacggacgccgtctgtatctgccggatatcaaatccagcaatggtgctcgtcgtgcagcggctgaacgtgcagccattaacgcgccaatgcagggaaccgccgccgacattatcaaacgggcgatgattgccgttgatgcgtggttacaggctgagcaaccgcgtgtacgtatgatcatgcaggtacacgatgaactggtatttgaagttcataaagatgatgttgatgccgtcgcgaagcagattcatcaactgatggaaaactgtacccgtctggatgtgccgttgctggtggaagtggggagtggcgaaaactgggatcaggcgcactaa

SEQ ID NO: 56 - protein, 681 aa, Sulfolobus acidocaldarius
  DNA_BIND (32)..(35)
  Linker (72)..(75)
MVHHHHHHKVKFKYKGEELQVDTSKIKKVWRVGKAISFTYDQGKTGRGAVSEKDAPKELLDMLARAEREKKGSAGMVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAH

SEQ ID NO: 57 - DNA, 1725 bp, Sulfolobus acidocaldarius; identical to SEQ ID NO: 21

SEQ ID NO: 58 - protein, 642 aa, Sulfolobus acidocaldarius
  DNA_BIND (26)..(29)
  Linker (66)..(69)
MVKVKFKYKGEELQVDTSKIKKVWRVGKAISFTYDQGKTGRGAVSEKDAPKELLDMLARAEREKKGSAGMKRYELKSILQKLFPDLEERENIEIKDVKEINFEEAKKEGCFAFKCLGEKGFEGISISFKEGEGYFIASFDFNDEVKGKVKDIISFENIKKIGAYIQRDLHFLDCKIKGEVFDVSLASYLLNPERQNHSLDILIREYLNRTSFIPQKYAAYLFPLKTILEERIKKEELEFVLFNIETPLIPVLYSMEKWGIKVDKEYLKSLSDEFCERIKKLEEEIYELAGMKFNLNSPKQLSEVLFERLKLPSGKKGKTGYSTSSLVLQNLLNAHPIVIKILQYRELYKLKSTYIDAIPNLINSQTGRVHTKFNPTGTATGRISSSEPNLQNIPIKSEEGRKIRRAFIADDGYYFVSLDYSQIELRIMAHLSQEPKLISAFQKGEDIHRRTAAEIFGVPEDEVDDLLRSRAKAVNFGIIYGISSFGLSETASITPEEAEKFIDSYFKHYPRVKLFIDKTIYEAREKLYVKTLFGRKRYIPEIRSINKQVRNAYERIAINAPIQGTAADIIKLAMIEIYKEIEEKNLKSRILLQIHDELILEVPEEEMEFTPLMAKEKMEKVVELSVPLVVEISVGKNLAELK

SEQ ID NO: 59 - DNA, 2496 bp, Escherichia coli
atggtgcatcaccatcaccatcatatttcttatgacaactacgtcaccatccttgatgaagaaacactgaaagcgtggattgcgaagctggaaaaagcgccggtatttgcatttgctaccgcaaccgacagccttgataacatctctgctaacctggtcgggctttcttttgctatcgagccaggcgtagcggcatatattccggttgctcatgattatcttgatgcgcccgatcaaatctctcgcgagcgtgcactcgagttgctaaaaccgctgctggaagatgaaaaggcgctgaaggtcgggcaaaacctgaaatacgatcgcggtattctggcgaactacggcattgaactgcgtgggattgcgtttgataccatgctggagtcctacattctcaatagcgttgccgggcgtcacgatatggacagcctcgcggaacgttggttgaagcacaaaaccatcacttttgaagagattgctggtaaaggcaaaaatcaactgacctttaaccagattgccctcgaagaagccggacgttacgccgccgaagatgcagatgtcaccttgcagttgcatctgaaaatgtggccggatctgcaaaaacacaaagggccgttgaacgtcttcgagaatatcgaaatgccgctggtgccggtgctttcacgcattgaacgtaacggtgtgaagatcgatccgaaagtgctgcacaatcattctgaagagctcacccttcgtctggctgagctggaaaagaaagcgcatgaaattgcaggtgaggaatttaacctttcttccaccaagcagttacaaaccattctctttgaaaaacagggcattaaaccgctgaagaaaacgccgggtggcgcgccgtcaacgtcggaagaggtactggaagaactggcgctggactatccgttgccaaaagtgattctggagtatcgtggtctggcgaagctgaaatcgacctacaccgacaagctgccgctgatgatcaacccgaaaaccgggcgtgtgcatacctcttatcaccaggcagtaactgcaacgggacgtttatcgtcaaccgatcctaacctgcaaaacattccggtgcgtaacgaagaaggtcgtcgtatccgccaggcgtttattgcgccagaggattatgtgattgtctcagcggactactcgcagattgaactgcgcattatggcgcatctttcgcgtgacaaaggcttgctgaccgcattcgcggaaggaaaagatatccaccgggcaacggcggcagaagtgtttggtttgccactggaaaccgtcaccagcgagcaacgccgtagcgcgaaagcgatcaactttggtctgatttatggcatgagtgctttcggtctggcgcggcaattgaacattccacgtaaagaagcgcagaagtacatggacctttacttcgaacgctaccctggcgtgctggagtatatggaacgcacccgtgctcaggcgaaagagcagggctacgttgaaacgctggacggacgccgtctgtatctgccggatatcaaatccagcaatggtgctcgtcgtgcagcggctgaacgtgcagccattaacgcgccaatgcagggaaccgccgccgacattatcaaacgggcgatgattgccgttgatgcgtggttacaggctgagcaaccgcgtgtacgtatgatcatgcaggtacacgatgaactggtatttgaagttcataaagatgatgttgatgccgtcgcgaagcagattcatcaactgatggaaaactgtacccgtctggatgtgccgttgctggtggaagtggggagtggcgaaaactgggatcaggcgcacggatccgcgggtatggcaagaggcctgaaccgcgtatacctcatcggctcccggcccgacatgcgctacaccccgggggggctcgagctcaacctggccgggcaggacaccctttgggaccaggagcgggaactcccctggtaccaccgggtgcggcgccaggcggagatgtggggggatgttttggagaagctcttcgtggagggaaggctggaataccgccagtggggggagaagcggagcgagctccaggtgcgggccgaccccttagacgcccgcgggcgggaaacccaggaggaccagccccgcctccgccacgccctgaaccaggtggtcaacctcacccgcgacgccgagctccgctacacccccgcggtggcccggctgggcctggcggtgaacgagcgcccgggggccgaggaggaaaaaacccatttcatagagtggcgcgaactggccgagtgggccggggagctcagggggcttttggtgatcggacgtttggtgaacgactcctccagcggggaaaggcgcttccagacccgcgtggaattggagcgacccacccgtgggcctgcccagaccggcccccaaccggtccagacgggtggggtggacattgacgaggacttcccgccggaggaggatctgccgttttga

SEQ ID NO: 60 - protein, 882 aa, Escherichia coli
  Linker (613)..(616)
MVHHHHHHISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHGSAGMARGLNRVYLIGSLTSRPDMRYTPGGLAILELNLAGQDTLWDESGQERELPWYHRVRLLGRQAEMWGDVLEKGQLLFAEGRLEYRQWERDGEKRSELQVRADFIDPLDARGRETQEDAKSQPRLRHALNQVVLMGNLTRDAELRYTPQGTAVARLGLAVNERRRGPGTEEEKTHFIEVQAWRELAEWAGELRKGDGLLVIGRLVNDSWTSSSGEGRFQTRVEALRLERPTRGPAQTGGSRPQPVQTGGVDIDEGLEDFPPEEDLPF

SEQ ID NO: 61 - DNA, 2577 bp, Thermus brockianus
atgggagaagatgggctatctttacctaagatgatgaatacaccaaaaccaattcttaaacctcaaccaaaagctttagtagaaccagtgctttgcgatagcattgatgaaataccagcgaaatataatgaaccagtatactttgccttggaaactgacgaagacagaccagttcttgcaagtatttatcaacctcactttgaacgcaaggtgtattgtttaaacctcttgaaagaaaaggtagcaaggtttaaagactggcttcttaaattctcagaaataagaggatggggtcttgactttgacttacgggttcttggctacacctacgaacaacttagaaacaagaagattgtagatgttcagcttgcgataaaagtccagcactacgagagatttaagcagggtgggaccaaaggtgaaggtttcagacttgatgatgtggcacgagatttgcttggtatagaatatccgatgaacaaaacaaaaattcgtgaaaccttcaaaaacaacatgtttcattcatttagcaacgaacaacttctttatgcctcgcttgatgcatacataccacacttgctttacgaacaactaacatcaagcacgcttaatagtcttgtttatcagcttgatcaacaggcacagaaagttgtgatagaaacatcgcaacacggcatgccagtaaaactaaaagcattagaagaagaaatacacagactaactcagctacgcagtgaaatgcaaaagcagataccatttaactataactctccaaaacaaacggcaaaattctttggagtaaatagttcttcaaaagatgtattgatggacttagctctacaaggaaatgaaatggctaaaaaggtgcttgaagcaagacaaatagaaaaatctcttgcttttgcaaaagacctctatgatatagctaaaagaagtggtggtagaatttacggcaacttctttactacaacagcaccatctggcagaatgtcttgctcggatataaatcttcaacagataccgcgtaggcttagatcattcataggctttgatacagaggacaaaaagcttatcaccgcagactttccgcaaattgagcttagacttgcaggtgtgatttggaatgaacctaaattcatagaagcatttaggcaaggtatagaccttcacaagcttacagcatcaatactgtttgataagaacatagaagaagtaagcaaggaagaaaggcaaattggaaaatctgcgaattatgggcttatctatggtattgcaccaaaaggtttcgcagaatattgtatagcgaacggtattaacatgacagaagagcaggcatacgaaatagtcagaaagtggaagaagtattacacaaagattgcagaacaacatcaagtagcatatgaaaggttcaaatacaatgagtatgtagataacgaaacatggcttaacagaacatatcgtgcatggaaaccacaagacctcttgaactatcaaatacaaggcagtggtgcggagctattcaagaaagctatagtattgttaaaagaaacaaagccagacttgaagatagtcaatctcgtgcatgatgagatagtagtagaagcagatagcaaagaagcacaagacttggctaagctaattaaagagaaaatggaggaagcgtgggattggtgtcttgaaaaagcagaagagtttggtaatagagttgctaaaataaaacttgaagtggaggagccacatgtgggtaatacatgggaaaagcctggatccgcgggtatggcaagaggcctgaaccgcgtatacctcatcggctccctcacctcccggcccgacatgcgctacaccccgggggggctcgccatcctggagctcaacctggccgggcaggacaccctttgggacgagtccggccaggagcgggaactcccctggtaccaccgggtgcggcttctgggccgccaggcggagatgtggggggatgttttggagaagggccagctcctcttcgcggagggaaggctggaataccgccagtgggagcgggacggggagaagcggagcgagctccaggtgcgggccgacttcattgaccccttagacgcccgcgggcgggaaacccaggaggacgccaagagccagccccgcctccgccacgccctgaaccaggtggtcctcatgggcaacctcacccgcgacgccgagctccgctacaccccccaggggacggcggtggcccggctgggcctggcggtgaacgagcgccgccgggggccggggaccgaggaggaaaaaacccatttcatagaggttcaggcctggcgcgaactggccgagtgggccggggagctcaggaagggcgacgggcttttggtgatcggacgtttggtgaacgactcctggacgagctccagcggggaagggcgcttccagacccgcgtggaagccctccgcttggagcgacccacccgtgggcctgcccagaccggcggaagcaggccccaaccggtccagacgggtggggtggacattgacgagggactcgaggacttcccgccggaggaggatctgccgttttga

SEQ ID NO: 62 - protein, 858 aa, Thermus brockianus
  Linker (589)..(592)
MGEDGLSLPKMMNTPKPILKPQPKALVEPVLCDSIDEIPAKYNEPVYFALETDEDRPVLASIYQPHFERKVYCLNLLKEKVARFKDWLLKFSEIRGWGLDFDLRVLGYTYEQLRNKKIVDVQLAIKVQHYERFKQGGTKGEGFRLDDVARDLLGIEYPMNKTKIRETFKNNMFHSFSNEQLLYASLDAYIPHLLYEQLTSSTLNSLVYQLDQQAQKVVIETSQHGMPVKLKALEEEIHRLTQLRSEMQKQIPFNYNSPKQTAKFFGVNSSSKDVLMDLALQGNEMAKKVLEARQIEKSLAFAKDLYDIAKRSGGRIYGNFFTTTAPSGRMSCSDINLQQIPRRLRSFIGFDTEDKKLITADFPQIELRLAGVIWNEPKFIEAFRQGIDLHKLTASILFDKNIEEVSKEERQIGKSANYGLIYGIAPKGFAEYCIANGINMTEEQAYEISQKVEEVLHKDCRQHQVAYERFKYNEYVDNETWLNRTYRAWKPQDLLNYQIQGSGAELFKKAIVLLKETKPDLKIVNLVHDEIVVEADSKEAQDLAKLIKEKMEEAWDWCLEKAEEFGNRVAKIKLEVEEPHVGNTWEKPGSAGMARGLNRVYLIGSLTSRPDMRYTPGGLAILELNLAGQDTLWDESGQERELPWYHRVRLLGRQAEMWGDVLEKGQLLFAEGRLEYRQWERDGEKRSELQVRADFIDPLDARGRETQEDAKSQPRLRHALNQVVLMGNLTRDAELRYTPQGTAVARLGLAVNERRRGPGTEEEKTHFIEVQAWRELAEWAGELRKGDGLLVIGRLVNDSWTSSSGEGRFQTRVEALRLERPTRGPAQTGGSRPQPVQTGGVDIDEGLEDFPPEEDLPF

SEQ ID NO: 63 - DNA, 3375 bp, Escherichia coli
atggggcatcaccatcaccatcacaaagaattttatatctctattgaaacagtcggaaataacattgttgaacgttatattgatgaaaatggaaaggaacgtacccgtgaagtagaatatcttccaactatgtttaggcattgtaaggaagagtcaaaatacaaagacatctatggtaaaaactgcgctcctcaaaaatttccatcaatgaaagatgctcgagattggatgaagcgaatggaagacatcggtctcgaagctctcggtatgaacgattttaaactcgcttatataagtgatacatatggttcagaaattgtttatgaccgaaaatttgttcgtgtagctaactgtgacattgaggttactggtgataaatttcctgacccaatgaaagcagaatatgaaattgatgctatcactcattacgattcaattgacgatcgtttttatgttttcgaccttttgaattcaatgtacggttcagtatcaaaatgggatgcaaagttagctgctaagcttgactgtgaaggtggtgatgaagttcctcaagaaattcttgaccgagtaatttatatgccattcgataatgagcgtgatatgctcatggaatatatcaatctttgggaacagaaacgacctgctatttttactggttggaatattgaggggtttgccgttccgtatatcatgaatcgtgttaaaatgattctgggtgaacgtagtatgaaacgtttctctccaatcggtcgggtaaaatctaaactaattcaaaatatgtacggtagcaaagaaatttattctattgatggcgtatctattcttgattatttagatttgtacaagaaattcgcttttactaatttgccgtcattctctttggaatcagttgctcaacatgaaaccaaaaaaggtaaattaccatacgacggtcctattaataaacttcgtgagactaatcatcaacgatacattagttataacatcattgacgtagaatcagttcaagcaatcgataaaattcgtgggtttatcgatctagttttaagtatgtcttattacgctaaaatgcctttttctggtgtaatgagtcctattaaaacttgggatgctattatttttaactcattgaaaggtgaacataaggttattcctcaacaaggttcgcacgttaaacagagttttccgggtgcatttgtgtttgaacctaaaccaattgcacgtcgatacattatgagttttgacttgacgtctctgtatccgagcattattcgccaggttaacattagtcctgaaactattcgtggtcagtttaaagttcatccaattcatgaatatatcgcaggaacagctcctaaaccgagtgatgaatattcttgttctccgaatggatggatgtatgataaacatcaagaaggtatcattccaaaggaaatcgctaaagtatttttccagcgtaaagactggaaaaagaaaatgttcgctgaagaaatgaatgccgaagctattaaaaagattattatgaaaggcgcagggtcttgttcaactaaaccagaagttgaacgatatgttaagttcagtgatgatttcttaaatgaactatcgaattacaccgaatctgttctcaatagtctgattgaagaatgtgaaaaagcagctacacttgctaatacaaatcagctgaaccgtaaaattctcattaacagtctttatggtgctcttggtaatattcatttccgttactatgatttgcgaaatgctactgctatcacaattttcggccaagtcggtattcagtggattgctcgtaaaattaatgaatatctgaataaagtatgcggaactaatgatgaagatttcattgcagcaggtgatactgattcggtatatgtttgcgtagataaagttattgaaaaagttggtcttgaccgattcaaagagcagaacgatttggttgaattcatgaatcagttcggtaagaaaaagatggaacctatgattgatgttgcatatcgtgagttatgtgattatatgaataaccgcgagcatctgatgcatatggaccgtgaagctatttcttgccctccgcttggttcaaagggcgttggtggattttggaaagcgaaaaagcgttatgctctgaacgtttatgatatggaagataagcgatttgctgaaccgcatctaaaaatcatgggtatggaaactcagcagagttcaacaccaaaagcagtgcaagaagctctcgaagaaagtattcgtcgtattcttcaggaaggtgaagagtctgtccaagaatactacaagaacttcgagaaagaatatcgtcaacttgactataaagttattgctgaagtaaaaactgcgaacgatatagcgaaatatgatgataaaggttggccaggatttaaatgcccgttccatattcgtggtgtgctaacttatcgtcgagctgttagcggtttaggtgtagctccaattttggatggaaataaagtaatggttcttccattacgtgaaggaaatccatttggtgacaagtgcattgcttggccatcgggtacagaacttccaaaagaaattcgttctgatgtgctatcttggattgaccactcaactttgttccaaaaatcgtttgttaaaccgcttgcgggtatgtgtgaatcggctggcatggactatgaagaaaaagcttcgttagacttcctgtttggcggatccgcgggtatggcaagaggcctgaaccgcgtatacctcatcggctcccggcccgacatgcgctacaccccgggggggctcgagctcaacctggccgggcaggacaccctttgggaccaggagcgggaactcccctggtaccaccgggtgcggcgccaggcggagatgtggggggatgttttggagaagctcttcgtggagggaaggctggaataccgccagtggggggagaagcggagcgagctccaggtgcgggccgaccccttagacgcccgcgggcgggaaacccaggaggaccagccccgcctccgccacgccctgaaccaggtggtcaacctcacccgcgacgccgagctccgctacacccccgcggtggcccggctgggcctggcggtgaacgagcgcccgggggccgaggaggaaaaaacccatttcatagagtggcgcgaactggccgagtgggccggggagctcagggggcttttggtgatcggacgtttggtgaacgactcctccagcggggaaaggcgcttccagacccgcgtggaattggagcgacccacccgtgggcctgcccagaccggcccccaaccggtccagacgggtggggtggacattgacgaggacttcccgccggaggaggatctgccgttttga

SEQ ID NO: 64 - protein, 1175 aa, Escherichia coli
  Linker (906)..(909)
MGHHHHHHKEFYISIETVGNNIVERYIDENGKERTREVEYLPTMFRHCKEESKYKDIYGKNCAPQKFPSMKDARDWMKRMEDIGLEALGMNDFKLAYISDTYGSEIVYDRKFVRVANCDIEVTGDKFPDPMKAEYEIDAITHYDSIDDRFYVFDLLNSMYGSVSKWDAKLAAKLDCEGGDEVPQEILDRVIYMPFDNERDMLMEYINLWEQKRPAIFTGWNIEGFAVPYIMNRVKMILGERSMKRFSPIGRVKSKLIQNMYGSKEIYSIDGVSILDYLDLYKKFAFTNLPSFSLESVAQHETKKGKLPYDGPINKLRETNHQRYISYNIIDVESVQAIDKIRGFIDLVLSMSYYAKMPFSGVMSPIKTWDAIIFNSLKGEHKVIPQQGSHVKQSFPGAFVFEPKPIARRYIMSFDLTSLYPSIIRQVNISPETIRGQFKVHPIHEYIAGTAPKPSDEYSCSPNGWMYDKHQEGIIPKEIAKVFFQRKDWKKKMFAEEMNAEAIKKIIMKGAGSCSTKPEVERYVKFSDDFLNELSNYTESVLNSLIEECEKAATLANTNQLNRKILINSLYGALGNIHFRYYDLRNATAITIFGQVGIQWIARKINEYLNKVCGTNDEDFIAAGDTDSVYVCVDKVIEKVGLDRFKEQNDLVEFMNQFGKKKMEPMIDVAYRELCDYMNNREHLMHMDREAISCPPLGSKGVGGFWKAKKRYALNVYDMEDKRFAEPHLKIMGMETQQSSTPKAVQEALEESIRRILQEGEESVQEYYKNFEKEYRQLDYKVIAEVKTANDIAKYDDKGWPGFKCPFHIRGVLTYRRAVSGLGVAPILDGNKVMVLPLREGNPFGDKCIAWPSGTELPKEIRSDVLSWIDHSTLFQKSFVKPLAGMCESAGMDYEEKASLDFLFGGSAGMARGLNRVYLIGSLTSRPDMRYTPGGLAILELNLAGQDTLWDESGQERELPWYHRVRLLGRQAEMWGDVLEKGQLLFAEGRLEYRQWERDGEKRSELQVRADFIDPLDARGRETQEDAKSQPRLRHALNQVVLMGNLTRDAELRYTPQGTAVARLGLAVNERRRGPGTEEEKTHFIEVQAWRELAEWAGELRKGDGLLVIGRLVNDSWTSSSGEGRFQTRVEALRLERPTRGPAQTGGSRPQPVQTGGVDIDEGLEDFPPEEDLPF

SEQ ID NO: 65 - DNA, 1992 bp, 3173 Thermostable Phage
atgggagaagatgggctatctttacctaagatgatgaatacaccaaaaccaattcttaaacctcaaccaaaagctttagtagaaccagtgctttgcgatagcattgatgaaataccagcgaaatataatgaaccagtatactttgccttggaaactgacgaagacagaccagttcttgcaagtatttatcaacctcactttgaacgcaaggtgtattgtttaaacctcttgaaagaaaaggtagcaaggtttaaagactggcttcttaaattctcagaaataagaggatggggtcttgactttgacttacgggttcttggctacacctacgaacaacttagaaacaagaagattgtagatgttcagcttgcgataaaagtccagcactacgagagatttaagcagggtgggaccaaaggtgaaggtttcagacttgatgatgtggcacgagatttgcttggtatagaatatccgatgaacaaaacaaaaattcgtgaaaccttcaaaaacaacatgtttcattcatttagcaacgaacaacttctttatgcctcgcttgatgcatacataccacacttgctttacgaacaactaacatcaagcacgcttaatagtcttgtttatcagcttgatcaacaggcacagaaagttgtgatagaaacatcgcaacacggcatgccagtaaaactaaaagcattagaagaagaaatacacagactaactcagctacgcagtgaaatgcaaaagcagataccatttaactataactctccaaaacaaacggcaaaattctttggagtaaatagttcttcaaaagatgtattgatggacttagctctacaaggaaatgaaatggctaaaaaggtgcttgaagcaagacaaatagaaaaatctcttgcttttgcaaaagacctctatgatatagctaaaagaagtggtggtagaatttacggcaacttctttactacaacagcaccatctggcagaatgtcttgctcggatataaatcttcaacagataccgcgtaggcttagatcattcataggctttgatacagaggacaaaaagcttatcaccgcagactttccgcaaattgagcttagacttgcaggtgtgatttggaatgaacctaaattcatagaagcatttaggcaaggtatagaccttcacaagcttacagcatcaatactgtttgataagaacatagaagaagtaagcaaggaagaaaggcaaattggaaaatctgcgaattatgggcttatctatggtattgcaccaaaaggtttcgcagaatattgtatagcgaacggtattaacatgacagaagagcaggcatacgaaatagtcagaaagtggaagaagtattacacaaagattgcagaacaacatcaagtagcatatgaaaggttcaaatacaatgagtatgtagataacgaaacatggcttaacagaacatatcgtgcatggaaaccacaagacctcttgaactatcaaatacaaggcagtggtgcggagctattcaagaaagctatagtattgttaaaagaaacaaagccagacttgaagatagtcaatctcgtgcatgatgagatagtagtagaagcagatagcaaagaagcacaagacttggctaagctaattaaagagaaaatggaggaagcgtgggattggtgtcttgaaaaagcagaagagtttggtaatagagttgctaaaataaaacttgaagtggaggagccacatgtgggtaatacatgggaaaagcctggatccgcgggtatggcacgtggtaaagtgaaatggttcgactccaagaaaggttacggcttcattactaaagatgaaggtggcgatgtgttcgtgcactggtccgcgattgaaatggaaggcttcaagaccctgaaagaaggtcaagtggttgaattcgagattcaagaaggcaagaaaggtccgcaagcagcgcatgttaaagtggttgaaggatccgcgggttga

SEQ ID NO: 66 - protein, 659 aa, 3173 Thermostable Phage
  Linker (589)..(592)
  RNA_BIND (606)..(610)
  RNA_BIND (618)..(622)
  DNA_BIND (624)..(627)
MGEDGLSLPKMMNTPKPILKPQPKALVEPVLCDSIDEIPAKYNEPVYFALETDEDRPVLASIYQPHFERKVYCLNLLKEKVARFKDWLLKFSEIRGWGLDFDLRVLGYTYEQLRNKKIVDVQLAIKVQHYERFKQGGTKGEGFRLDDVARDLLGIEYPMNKTKIRETFKNNMFHSFSNEQLLYASLDAYIPHLLYEQLTSSTLNSLVYQLDQQAQKVVIETSQHGMPVKLKALEEEIHRLTQLRSEMQKQIPFNYNSPKQTAKFFGVNSSSKDVLMDLALQGNEMAKKVLEARQIEKSLAFAKDLYDIAKRSGGRIYGNFFTTTAPSGRMSCSDINLQQIPRRLRSFIGFDTEDKKLITADFPQIELRLAGVIWNEPKFIEAFRQGIDLHKLTASILFDKNIEEVSKEERQIGKSANYGLIYGIAPKGFAEYCIANGINMTEEQAYEIVRKWKKYYTKIAEQHQVAYERFKYNEYVDNETWLNRTYRAWKPQDLLNYQIQGSGAELFKKAIVLLKETKPDLKIVNLVHDEIVVEADSKEAQDLAKLIKEKMEEAWDWCLEKAEEFGNRVAKIKLEVEEPHVGNTWEKPGSAGMARGKVKWFDSKKGYGFITKDEGGDVFVHWSAIEMEGFKTLKEGQVVEFEIQEGKKGPQAAHVKVVE

SEQ ID NO: 67 - DNA, 2187 bp, Thermotoga maritima
atggcacgtggtaaagtgaaatggttcgactccaagaaaggttacggcttcattactaaagatgaaggtggcgatgtgttcgtgcactggtccgcgattgaaatggaaggcttcaagaccctgaaagaaggtcaagtggttgaattcgagattcaagaaggcaagaaaggtccgcaagcagcgcatgttaaagtggttgaaggatccgcgggtatgggagaagatgggctatctttacctaagatgatgaatacaccaaaaccaattcttaaacctcaaccaaaagctttagtagaaccagtgctttgcgatagcattgatgaaataccagcgaaatataatgaaccagtatactttgccttggaaactgacgaagacagaccagttcttgcaagtatttatcaacctcactttgaacgcaaggtgtattgtttaaacctcttgaaagaaaaggtagcaaggtttaaagactggcttcttaaattctcagaaataagaggatggggtcttgactttgacttacgggttcttggctacacctacgaacaacttagaaacaagaagattgtagatgttcagcttgcgataaaagtccagcactacgagagatttaagcagggtgggaccaaaggtgaaggtttcagacttgatgatgtggcacgagatttgcttggtatagaatatccgatgaacaaaacaaaaattcgtgaaaccttcaaaaacaacatgtttcattcatttagcaacgaacaacttctttatgcctcgcttgatgcatacataccacacttgctttacgaacaactaacatcaagcacgcttaatagtcttgtttatcagcttgatcaacaggcacagaaagttgtgatagaaacatcgcaacacggcatgccagtaaaactaaaagcattagaagaagaaatacacagactaactcagctacgcagtgaaatgcaaaagcagataccatttaactataactctccaaaacaaacggcaaaattctttggagtaaatagttcttcaaaagatgtattgatggacttagctctacaaggaaatgaaatggctaaaaaggtgcttgaagcaagacaaatagaaaaatctcttgcttttgcaaaagacctctatgatatagctaaaagaagtggtggtagaatttacggcaacttctttactacaacagcaccatctggcagaatgtcttgctcggatataaatcttcaacagataccgcgtaggcttagatcattcataggctttgatacagaggacaaaaagcttatcaccgcagactttccgcaaattgagcttagacttgcaggtgtgatttggaatgaacctaaattcatagaagcatttaggcaaggtatagaccttcacaagcttacagcatcaatactgtttgataagaacatagaagaagtaagcaaggaagaaaggcaaattggaaaatctgcgaattatgggcttatctatggtattgcaccaaaaggtttcgcagaatattgtatagcgaacggtattaacatgacagaagagcaggcatacgaaatagtcagaaagtggaagaagtattacacaaagattgcagaacaacatcaagtagcatatgaaaggttcaaatacaatgagtatgtagataacgaaacatggcttaacagaacatatcgtgcatggaaaccacaagacctcttgaactatcaaatacaaggcagtggtgcggagctattcaagaaagctatagtattgttaaaagaaacaaagccagacttgaagatagtcaatctcgtgcatgatgagatagtagtagaagcagatagcaaagaagcacaagacttggctaagctaattaaagagaaaatggaggaagcgtgggattggtgtcttgaaaaagcagaagagtttggtaatagagttgctaaaataaaacttgaagtggaggagccacatgtgggtaatacatgggaaaagcctggatccgcgggtatggtgaaggttaaattcaagtataagggtgaggagctgcaagtggacacttccaagattaagaaagtgtggcgtgttggcaaggcgatttcctttacctacgaccaaggtaagaccggtcgcggtgcggtttcggagaaagacgcaccaaaggagctgttggacatgctggcacgtgcggaacgcgagaagaaatga

SEQ ID NO: 68 - protein, 729 aa, Thermotoga maritima
  RNA_BIND (14)..(18)
  RNA_BIND (26)..(30)
  DNA_BIND (32)..(35)
  Linker (68)..(71)
  Linker (660)..(663)
  DNA_BIND (689)..(692)
MARGKVKWFDSKKGYGFITKDEGGDVFVHWSAIEMEGFKTLKEGQVVEFEIQEGKKGPQAAHVKVVEGSAGMGEDGLSLPKMMNTPKPILKPQPKALVEPVLCDSIDEIPAKYNEPVYFALETDEDRPVLASIYQPHFERKVYCLNLLKEKVARFKDWLLKFSEIRGWGLDFDLRVLGYTYEQLRNKKIVDVQLAIKVQHYERFKQGGTKGEGFRLDDVARDLLGIEYPMNKTKIRETFKNNMFHSFSNEQLLYASLDAYIPHLLYEQLTSSTLNSLVYQLDQQAQKVVIETSQHGMPVKLKALEEEIHRLTQLRSEMQKQIPFNYNSPKQTAKFFGVNSSSKDVLMDLALQGNEMAKKVLEARQIEKSLAFAKDLYDIAKRSGGRIYGNFFTTTAPSGRMSCSDINLQQIPRRLRSFIGFDTEDKKLITADFPQIELRLAGVIWNEPKFIEAFRQGIDLHKLTASILFDKNIEEVSKEERQIGKSANYGLIYGIAPKGFAEYCIANGINMTEEQAYEIVRKWKKYYTKIAEQHQVAYERFKYNEYVDNETWLNRTYRAWKPQDLLNYQIQGSGAELFKKAIVLLKETKPDLKIVNLVHDEIVVEADSKEAQDLAKLIKEKMEEAWDWCLEKAEEFGNRVAKIKLEVEEPHVGNTWEKPGSAGMVKVKFKYKGEELQVDTSKIKKVWRVGKAVSFTYDDNGKTGRGAVSEKDAPKELLDMLARAEREKK

SEQ ID NO: 69 - DNA, 207 bp, Chimeric
atggttaaagttaaatttaaatataaaggttatggttttattactaaagatgaaggtggtgatgtttttgttcattggcgtgttggtaaagctgtttcttttacttatgatgataatggtaaaactggtcgtggtgctgtttctgaaaaagatgctcctaaagaacttcttgatatgcttgctcgtgctgaacgtgaaaaaaaatga

SEQ ID NO: 70 - protein, 68 aa, Chimeric
  RNA_BIND (10)..(14)
  RNA_BIND (22)..(26)
  DNA_BIND (28)..(31)
MVKVKFKYKGYGFITKDEGGDVFVHWRVGKAVSFTYDDNGKTGRGAVSEKDAPKELLDMLARAEREKK

SEQ ID NO: 71 - DNA, 1983 bp, Chimeric
atggttaaagttaaatttaaatataaaggttatggttttattactaaagatgaaggtggtgatgtttttgttcattggcgtgttggtaaagctgtttcttttacttatgatgataatggtaaaactggtcgtggtgctgtttctgaaaaagatgctcctaaagaacttcttgatatgcttgctcgtgctgaacgtgaaaaaaaaggatccgcgggtatgggagaagatgggctatctttacctaagatgatgaatacaccaaaaccaattcttaaacctcaaccaaaagctttagtagaaccagtgctttgcgatagcattgatgaaataccagcgaaatataatgaaccagtatactttgccttggaaactgacgaagacagaccagttcttgcaagtatttatcaacctcactttgaacgcaaggtgtattgtttaaacctcttgaaagaaaaggtagcaaggtttaaagactggcttcttaaattctcagaaataagaggatggggtcttgactttgacttacgggttcttggctacacctacgaacaacttagaaacaagaagattgtagatgttcagcttgcgataaaagtccagcactacgagagatttaagcagggtgggaccaaaggtgaaggtttcagacttgatgatgtggcacgagatttgcttggtatagaatatccgatgaacaaaacaaaaattcgtgaaaccttcaaaaacaacatgtttcattcatttagcaacgaacaacttctttatgcctcgcttgatgcatacataccacacttgctttacgaacaactaacatcaagcacgcttaatagtcttgtttatcagcttgatcaacaggcacagaaagttgtgatagaaacatcgcaacacggcatgccagtaaaactaaaagcattagaagaagaaatacacagactaactcagctacgcagtgaaatgcaaaagcagataccatttaactataactctccaaaacaaacggcaaaattctttggagtaaatagttcttcaaaagatgtattgatggacttagctctacaaggaaatgaaatggctaaaaaggtgcttgaagcaagacaaatagaaaaatctcttgcttttgcaaaagacctctatgatatagctaaaagaagtggtggtagaatttacggcaacttctttactacaacagcaccatctggcagaatgtcttgctcggatataaatcttcaacagataccgcgtaggcttagatcattcataggctttgatacagaggacaaaaagcttatcaccgcagactttccgcaaattgagcttagacttgcaggtgtgatttggaatgaacctaaattcatagaagcatttaggcaaggtatagaccttcacaagcttacagcatcaatactgtttgataagaacatagaagaagtaagcaaggaagaaaggcaaattggaaaatctgcgaattatgggcttatctatggtattgcaccaaaaggtttcgcagaatattgtatagcgaacggtattaacatgacagaagagcaggcatacgaaatagtcagaaagtggaagaagtattacacaaagattgcagaacaacatcaagtagcatatgaaaggttcaaatacaatgagtatgtagataacgaaacatggcttaacagaacatatcgtgcatggaaaccacaagacctcttgaactatcaaatacaaggcagtggtgcggagctattcaagaaagctatagtattgttaaaagaaacaaagccagacttgaagatagtcaatctcgtgcatgatgagatagtagtagaagcagatagcaaagaagcacaagacttggctaagctaattaaagagaaaatggaggaagcgtgggattggtgtcttgaaaaagcagaagagtttggtaatagagttgctaaaataaaacttgaagtggaggagccacatgtgggtaatacatgggaaaagccttga

SEQ ID NO: 72 - protein, 660 aa, Chimeric
  RNA_BIND (10)..(14)
  RNA_BIND (22)..(26)
  DNA_BIND (28)..(31)
  Linker (69)..(72)
MVKVKFKYKGYGFITKDEGGDVFVHWRVGKAVSFTYDDNGKTGRGAVSEKDAPKELLDMLARAEREKKGSAGMGEDGLSLPKMMNTPKPILKPQPKALVEPVLCDSIDEIPAKYNEPVYFALETDEDRPVLASIYQPHFERKVYCLNLLKEKVARFKDWLLKFSEIRGWGLDFDLRVLGYTYEQLRNKKIVDVQLAIKVQHYERFKQGGTKGEGFRLDDVARDLLGIEYPMNKTKIRETFKNNMFHSFSNEQLLYASLDAYIPHLLYEQLTSSTLNSLVYQLDQQAQKVVIETSQHGMPVKLKALEEEIHRLTQLRSEMQKQIPFNYNSPKQTAKFFGVNSSSKDVLMDLALQGNEMAKKVLEARQIEKSLAFAKDLYDIAKRSGGRIYGNFFTTTAPSGRMSCSDINLQQIPRRLRSFIGFDTEDKKLITADFPQIELRLAGVIWNEPKFIEAFRQGIDLHKLTASILFDKNIEEVSKEERQIGKSANYGLIYGIAPKGFAEYCIANGINMTEEQAYEIVRKWKKYYTKIAEQHQVAYERFKYNEYVDNETWLNRTYRAWKPQDLLNYQIQGSGAELFKKAIVLLKETKPDLKIVNLVHDEIVVEADSKEAQDLAKLIKEKMEEAWDWCLEKAEEFGNRVAKIKLEVEEPHVGNTWEKP

SEQ ID NO: 73 - DNA, 26 bp, Artificial Sequence
  PCR primer
tgagccagtgagttgattgcagtcca

SEQ ID NO: 74 - DNA, 26 bp, Artificial Sequence
  PCR primer
gaagcgggtttttaccttatttgcgg

SEQ ID NO: 75 - DNA, 24 bp, Artificial Sequence
  PCR primer
gaagaggtggcgcgtaacgcgtcc

SEQ ID NO: 76 - DNA, 25 bp, Artificial Sequence
  PCR primer
gatgacatgcttgtttcatcaggtg

SEQ ID NO: 77 - DNA, 24 bp, Artificial Sequence
  PCR primer
cgccagggttttcccagtcacgac

SEQ ID NO: 78 - DNA, 18 bp, Artificial Sequence
  PCR primer
agatccgcacgcacaacc

SEQ ID NO: 79 - DNA, 22 bp, Artificial Sequence
  PCR primer
cctgctcgctctctcaatctct

SEQ ID NO: 80 - DNA, 18 bp, Artificial Sequence
  PCR primer
ctggtctggccctgatgg

SEQ ID NO: 81 - DNA, 18 bp, Artificial Sequence
  PCR primer
cctggacgccctaacctg